(19) United States
(12) Patent Application Publication
(10) Pub. No.: US 2024/0216436 A1
(43) Pub. Date: Jul. 4, 2024

(54) INTERFERON GAMMA-PRIMED MESENCHYMAL STROMAL CELLS AS PROPHYLAXIS FOR GRAFT VERSUS HOST DISEASE

(71) Applicants: Ossium Health, Inc., San Francisco, CA (US); Emory University, Atlanta, GA (US); Children's Healthcare of Atlanta, Atlanta, GA (US)

(72) Inventors: Edwin M. HORWITZ, Atlanta, GA (US); Erik J. WOODS, SR., Carmel, IN (US); Brian H. JOHNSTONE, Fishers, IN (US); Hannah Marie MILLER, Speedway, IN (US)

(21) Appl. No.: 18/486,907

(22) Filed: Oct. 13, 2023

Related U.S. Application Data
(63) Continuation of application No. PCT/US2022/025040, filed on Apr. 15, 2022.
(60) Provisional application No. 63/238,048, filed on Aug. 27, 2021.
(60) Provisional application No. 63/176,182, filed on Apr. 16, 2021.

(51) Int. Cl. A61K 35/28; A61P 37/06

(52) U.S. Cl. CPC A61K 35/28; A61P 37/06

(57) ABSTRACT

The present disclosure provides composition and methods comprising interferon γ-primed human mesenchymal stromal cells (γMSCs) for preventing or reducing the likelihood of Graft Versus Host Disease (GVHD) or a symptom thereof in a human subject that has been administered a hematopoietic stem cell transplant (HCT).

### CROSS-REFERENCE

This application is a continuation of International Application No. PCT/US2022/025040, filed Apr. 15, 2022, which claims the benefit of U.S. Provisional Application No. 63/176,182, filed Apr. 16, 2021 and U.S. Provisional Application No. 63/238,048, filed Aug. 27, 2021. The entire contents of each of which is incorporated by reference in its entirety.

### BACKGROUND

Hematopoietic cell transplantation (HCT) is an established therapeutic modality for high risk hematological malignancies in adults and children. The primary cause of morbidity and mortality after HCT is graft versus host disease (GVHD), affecting up to 70% of patients even with current prophylaxis. GVHD directly accounts for approximately a third of regimen-related deaths. Currently, pharmacologic GVHD prophylaxis and therapeutics are administered to prevent or treat GVHD; however, these are only partially effective, increase the risk of infection and disease relapse and impart drug-related, short- and long-term adverse effects.

### SUMMARY

Mesenchymal stromal cells (MSCs) have potent immune modulatory activity which is markedly enhanced by exposure to interferon γ (IFNγ). In murine models, IFNγ primed MSCs (γMSCs) suppress GVHD without untoward adverse effects, suggesting this cell therapy may markedly reduce HCT-related GVHD. γMSCs have never been infused into human patients and their effectiveness in humans for reducing GVHD have not been determined. Therefore, there is an unmet need for methods and systems of safely manufacturing and administering γMSCs in human patients who are undergoing HCT and are at risk for GVHD. The present disclosure describes systems and methods of manufacturing and administering γMSCs to human patients; as disclosed herein, administering γMSCs provides potent GVHD prophylactic activity.

An aspect of the present disclosure is a composition comprising interferon γ-primed human mesenchymal stromal cells (γMSCs) for preventing or reducing the likelihood of Graft Versus Host Disease (GVHD) or a symptom thereof in a human subject that has been administered a hematopoietic stem cell transplant (HCT). When the MSCs were being interferon γ-primed, the MSCs were not in a culture in a hypoxic condition.

Another aspect of the present disclosure is a method for preventing or reducing the likelihood of Graft Versus Host Disease (GVHD) or a symptom thereof in a subject that has been administered a hematopoietic stem cell transplant (HCT). The method comprises administering to the subject an effective amount of interferon γ-primed mesenchymal stromal cells (γMSCs). When the MSCs were being interferon γ-primed, the MSCs were not in a culture in a hypoxic condition.

Yet another method of the present disclosure is a method for preventing or reducing the likelihood of Graft Versus Host Disease (GVHD) or a symptom thereof in a subject. The method comprising steps of administering to the subject a hematopoietic stem cell transplant (HCT); and administering to the subject an effective amount of interferon γ-primed mesenchymal stromal cells (γMSCs). When the MSCs were being interferon γ-primed, the MSCs were not in a culture in a hypoxic condition.

In an aspect, the present disclosure provides a method for preventing or reducing the likelihood of Graft Versus Host Disease (GVHD) or a symptom thereof in a subject that will be administered a hematopoietic stem cell transplant (HCT). The method comprising administering to the subject an effective amount of interferon γ-primed mesenchymal stromal cells (γMSCs). When the MSCs were being interferon γ-primed, the MSCs were not in a culture in a hypoxic condition.

In another aspect, the present disclosure provides a method for manufacturing interferon γ-primed mesenchymal stromal cells (γMSCs), the method comprising obtaining a culture of MSCs and contacting the MSCs with interferon gamma (IFNγ). When the MSCs were being interferon γ-primed, the MSCs were not in a culture in a hypoxic condition.

In yet another aspect, the present disclosure provides a use of any herein disclosed composition in a method for preventing or reducing the likelihood of Graft Versus Host Disease (GVHD) or a symptom thereof.

An aspect of the present disclosure is a plurality of herein disclosed compositions and uses thereof.

In any of the herein disclosed aspects or embodiments, rather than cryopreserving MSCs or γMSCs and thawing the MSCs (which are subsequently primed) or thawing the γMSCs prior to use (either immediately or after one or more culturing steps), fresh MSCs or fresh γMSCs may be used.

Any herein disclosed aspect or embodiment can be combined with any other aspect or embodiment.

### BRIEF DESCRIPTION OF THE DRAWINGS

FIG. 1A to FIG. 1D are schematics showing illustrative manufacturing processes for interferon γ-primed mesenchymal stromal cells (γMSCs).

FIG. 2 is a flow diagram of product process and controls.

FIG. 3 is a graph demonstrating differential gene expression of MSCs (open shapes) and γ-MSC (filled shapes) from each donor (same color and at similar verticality).

FIG. 4 is a graph showing Euclidean distance calculated to assess the overall similarity between samples.

FIG. 5A and FIG. 5B show representative karyotypes of MSCs (FIG. 5A) and γ-IFN-primed MSCs (FIG. 5B) from cells that have undergone primary expansion, cryopreservation, and secondary expansion, and are appropriate to infuse into a subject.

FIG. 6A to FIG. 6E show phenotypic and functional characterization of vBA-MSC. FIG. 6A shows expression of MHC and other surface markers with or without IFNγ for 2 days. FIG. 6B shows a chemotaxis assay showing increased number of MSCs in lower chamber in response to increasing amounts of CCL19, one of the CCR7 ligands. FIG. 6C are histograms showing flow cytometric analyses of interferon γ primed BM-MSCs for the indicated antigens (blue solid line) or isotype control (red dashed line). FIG. 6D are scatter plots showing three γ primed BM-MSCs expansions showing percentages of CD45 and CD105 cells. FIG. 6E are flow cytometric histograms showing the dilution of cell trace far red membrane dye (to the left) with expansion compared with a uniform bright signal (to the right) of nondividing cells.

FIG. 7 is a graph illustrating survival percentages of mice administered murine γMSCs and human γMSCs.

FIG. 8 is a graph illustrating survival percentages of mice administered a single infusion of γMSCs as compared to mice administered two infusions of γMSCs.

FIG. 9 is a flowchart illustrating a dose escalation schema used for determining an effective dosage of γMSCs for human administration.

### DETAILED DESCRIPTION

Hematopoietic cell transplantation (HCT) is an established therapeutic for high risk hematological malignancies in adults and children. The hematopoietic stem cells are usually derived from bone marrow, peripheral blood, or umbilical cord blood. HCT is commonly used for patients with certain cancers of the blood or bone marrow, or for patients receiving an organ transplant. In these instances, graft versus host disease (GVHD) is commonly a major complication.

The primary cause of morbidity and mortality after HCT is graft versus host disease (GVHD), which affects up to 70% of patients even with current pharmacological prophylaxis and/or therapeutics. GVHD directly accounts for approximately a third of regimen-related deaths. When HCT or an organ transplant occurs, white blood cells of the donor's immune system that remain in the donated tissue (the graft) recognize the recipient (the host) as foreign. The white blood cells in the graft attack the host, causing GVHD.

Mesenchymal stromal cells (MSCs) have potent immune modulatory activity, including prophylactic activity of GVHD, which is markedly enhanced by exposure to IFNγ. In vivo, MSCs are stimulated in response to abnormal conditions, e.g., caused by infection, cancer, and injury. The presence of circulating IFNγ can be a signal to MSCs which identifies an abnormal condition. In response to the circulating IFNγ, the MSCs are primed and secrete, at least, factors and proteins that help remedy the abnormal condition. In various aspects of the present disclosure, MSCs are primed in vitro or ex vivo by contact with IFNγ to transform the cells into γMSCs.

In murine models, IFNγ primed MSCs, (γMSCs), potently suppress GVHD without untoward adverse effects, suggesting γMSCs therapy may markedly reduce the toxicity resulting from HCT. However, γMSCs have never been infused into human patients. The present disclosure describes systems and methods of manufacturing and administering γMSCs to human patients; as disclosed herein, administering γMSCs provides potent GVHD prophylactic activity. In certain embodiments when the MSCs were being interferon γ-primed, the MSCs were not in a culture under in a hypoxic condition.

### Preparing the Donor Bone

In various embodiments, an Organ Procurement Organization (OPOs) performs evaluation and donor recoveries according to 21 CFR § 1271. A donor screening and monitoring process may include completion and assessment of a Uniform Donor Risk Assessment Interview (UDRAI). The UDRAI comprises of flowcharts, guidance documents, and questionnaires which are used to screen potential donors related to medical history, behavioral history, travel history and social history. Serology testing may be completed in a CLIA/CMS-approved laboratory using FDA-cleared test kits to rule out viral pathogens. Donors should be negative or non-reactive for HIV-1, HIV-2, hepatitis B virus (HBV, surface and core antigen), hepatitis C virus (HCV), Syphilis (Treponema pallidum), human T-lymphotropic virus types 1 and 2 (HTLV-1, HTLV-2), West Nile Virus, Chagas (Trypanosoma cruzi), Toxoplasmosis, and Epstein-Barr Virus (EBV). Donors may be screened for Cytomegalovirus (CMV). In some embodiments, a donor is under 30 years old, non-smoker, and confirmed CMV negative.

In some embodiments, the donor bone is vertebral bodies. However, it is understood that the methods described herein can be used on the ilium, a combination of the vertebral bodies and ilium, or other bones suitable for extraction of MSCs, even donor bones with lower expected yields.

It is understood that the donor bones can be procured according to fixed protocols for clinical recovery. Bones can be recovered by surgeons or by personnel at a trained OPO (organ procurement organization) using an osteotome and mallet from consented organ and tissue donors. Unprocessed bones are preferably wrapped in sponges and towels soaked in saline to ensure moisture retention during hypothermic shipment on wet ice at a temperature of 0 to 10° F. to a processing facility.

The process for preparing the donor bone can occur soon after the bone is obtained from the deceased donor or can occur after the donor bone has been shipped in a hypothermic environment to a processing facility. Since the donor bone can experience prolonged periods of ischemia during recovery and shipment to the processing facility, care must be taken to track the length and type of ischemia—i.e., warm ischemia and cold ischemia. As described in more detail herein, bone subject to predetermined periods of warm and/or cold ischemia are suitable for obtaining meaningful quantities of viable bone marrow cells.

During the processing of the donor bone, the bone is debrided in an ISO-5 (class 100) environment (biosafety cabinet) with an ISO-7 (class 10,000) background (clean room), with special care taken to sterilize the bag containing the donor bone, such as by spraying with 70% isopropanol. In one embodiment, the debridement is conducted manually using scalpels, osteotomes and gouges. In processing vertebrae, typically a spinal segment including multiple vertebral levels will be provided. In a typical case, the spine segment runs from T8 to L5, for ten vertebral bodies. During initial debridement of the spinal segment, when enough soft tissue has been removed to visualize the pedicles, the pedicles are removed using either a tissue processing band saw or a bone saw, such as the Stryker System 6 Saw (Stryker, Kalamazoo, MI). Special care is taken to avoid breaching the cortical bone which would expose the cancellous bone, to ensure that the hypoxic cancellous bone marrow remains protected throughout the entire debriding process. The anterior element of the vertebral bodies remain, while the pedicles and posterior elements are discarded.

Using a boning knife or tissue processing band saw, the vertebral bodies are separated at the intervertebral discs. The intervertebral disc and soft tissue remaining on each vertebral body is removed with a scalpel, scissors and/or osteotomes, leaving clean, separated VBs. In the case of donor ilium, the soft tissue can be removed with gouges and a scalpel, with special care again taken to ensure that the cortical bone is not breached. Any anatomical pathologies or injuries of the bone are noted and recorded as part of the batch record for the marrow ultimately obtained from the bones. Bones damaged during the recovery process are discarded.

The VBs are placed into a sterile bag and submerged in a 10% bleach solution, yielding a concentration of 5,000 ppm free chlorine, for a predetermined period, typically 5 or more minutes. Bleach has a broad spectrum of anti-microbial activity, does not leave a toxic residue, is unaffected by water hardness and is fast acting. At the end of the period, the bones are transferred to another sterile bag and submerged in a 3% hydrogen peroxide (H₂O₂) solution. The bag is closed and shaken briefly to ensure that the entire surface of the bone is in contact with the solution. Most living cells include catalase, which is an enzyme that catalyzes the breakdown of H₂O₂ into H₂O and O₂. This breakdown manifests as foam or froth when the H₂O₂ solution contacts soft tissue but not bone. The foam level can be observed as an indication of the amount of soft tissue remaining on the bone. This observation can be performed manually by a human processor or, in another embodiment, by an automated processor. The automated processor incorporates a visualization device, such as a camera, and object recognition software that can determine foam levels within the bag. The addition of an inert contrast dye can help the human or automated processor detect the foam level. If any foam or froth is observed, the bone is returned for further processing to remove all of the remaining soft tissue from the bone. Once the VBs or ilium has been cleaned of all soft tissue, the bones are transferred to a new sterile bag. The bag is filled with 1 L of PLASMA-LYTE™ (multiple electrolytes injection obtained from Baxter Healthcare, Ltd.), or other suitable sterile, nonpyrogenic isotonic solution. The bag is closed and shaken briefly to ensure that the entire bone is contacted with the PLASMA-LYTE™.

Bone marrow from each group of VBs processed at different duration of bleach treatment can be tested by flow cytometry to assess the viability of the cells isolated from the bone marrow (Table 1). As seen from Table, soaking the VBs for more than 10 minutes yields no significant difference in cell viability compared to when the VBs are soaked for up to 25 minutes.

TABLE 1
Bleach Soak of Vertebral Bodies

| | CD45+ | CD34+ | CD3+ |
|---|---|---|---|
| Time Point (min) | % Viability | % Viability | % Viability |
| 10 | 88.59 | 95.92 | 80.50 |
| 15 | 88.78 | 97.95 | 75.61 |
| 20 | 87.12 | 96.85 | 71.14 |
| 25 | 86.81 | 96.75 | 71.67 |
| Avg. | 87.825 | 96.8675 | 74.73 |
| SD | 0.869554 | 0.721747 | 3.752299 |

In some embodiments, the bleach treatment comprises using 1%, 2%, 3%, 4%, 5%, 6%, 7%, 8%, 9%, 10%, 11%, 12%, 13%, 14%, 15%, 20%, 25%, 30%, 35%, 40%, 45%, 50%, or higher percentage of bleach. In some embodiments, the bleach treatment comprises contacting the VBs with bleach for at least 1 minute, 2 minutes, 3 minutes, 4 minutes, 5 minutes, 6 minutes, 7 minutes, 8 minutes, 9 minutes, 10 minutes, 11, minutes, 12 minutes, 13 minutes, 14 minutes, 15 minutes, 20 minutes, 25 minutes, 30 minutes, 35 minutes, 40 minutes, 45 minutes, 50 minutes, or longer duration. In some embodiments, the viability of the bone marrow cells isolated from the VBs treated with the bleach treatment is not significantly decreased at any duration of bleach treatment described herein compared to bone marrow cells isolated from the VBs without the bleach treatment. In some embodiments, the viability of the bone marrow cells isolated from the VBs treated with 11 minutes, 12 minutes, 13 minutes, 14 minutes, 15 minutes, 20 minutes, 25 minutes, 30 minutes, 35 minutes, 40 minutes, 45 minutes, 50 minutes, or longer duration of the bleach treatment is not decreased or is decreased by less than 3% compared to the viability of the bone marrow cells isolated from the VBs treated with the 10 minutes bleach treatment. In some embodiments, the viability of the bone marrow cells isolated from the VBs treated with more than 10 minutes decreased by less than 2% compared to the viability of the bone marrow cells isolated from the VBs treated with the 10 minutes bleach treatment. In some embodiments, the viability of the bone marrow cells isolated from the VBs treated with more than 10 minutes decreased by less than T % compared to the viability of the bone marrow cells isolated from the VBs treated with the 10 minutes bleach treatment.

The bone is removed from the bag and from the PLASMA-LYTE™, and a sterile gauze or sponge is used to absorb any liquid remaining on the VBs. In one approach, a saw and/or anvil shears are used to cut the VBs are cut into smaller pieces, such as 1.5 cm² pieces, that are small enough for fragmenting with a bone grinder. In order to simplify the process and for increased safety to the processing personnel, a custom bone cutting tool as described in WO2020214400A1, which is hereby incorporated by reference in its entirety, is provided is used to cut the VBs into the smaller pieces.

Methods of Deriving Hematopoietic Stem Cells from Bone Marrow

A fresh cadaver vertebral body (VB) or a warmed, previously cryopreserved VB is prepared for grinding.

In one approach, a saw and/or anvil shears are used to cut the VBs are cut into smaller pieces, such as 1.5 cm² pieces, that are small enough for fragmenting with a bone grinder. In order to simplify the process and for increased safety to the processing personnel, a custom bone cutting tool as described in US 2019/0343112, which is hereby incorporated by reference in its entirety, is provided is used to cut the VBs into the smaller pieces. Another custom bone cutting tool can be used in combination, or in lieu of the custom bone cutting tool as described in US 2019/0343112. The additional bone cutting tool is described in US20200325451, which is hereby incorporated by reference in its entirety.

The elements of the bone cutting tool are formed of medical grade stainless steel. The steel is preferably hardened steel capable of withstanding the forces required to cut through bone. In the cleaning process, the tool is subjected to steam sterilization, which can be deleterious to the steel. Thus, in one feature of the present disclosure, the surfaces of the stainless-steel elements are passivated to prevent oxidation of the steel elements during sterilization.

The pieces produced by the bone cutting tool are immediately placed into a sterile pitcher and submerged in 300-500 ml of a grind media. In one aspect of the present system and method, the grind media uses PLASMA-LYTE™-A as a base with heparin, human serum albumin (HSA), and a nuclease (Merck KGAA Corporation). Heparin is used as an anticoagulant. Other anticoagulants at various quantities can also be used. HSA provides a protein source to prevent cell adherence and adsorption to surfaces, as well as reactive oxygen scavenging. It is noted that conventional grind media utilizes DNase, but for the present disclosure Benzonase® or Denarase® reagent is substituted for DNase™ reagent (Qiagen Sciences LLC). Whereas DNase works only on DNA, modem pharmaceutical biotechnology processing relies on enzymes that can cleave all forms of DNA and RNA, and can reduce the viscosity of the solution in which the cells are suspended. It is noted that IMDM (Iscove's Modified Dulbecco's Media) can substitute for the PLASMA-LYTE™-A, since IMDM is suitable for rapidly proliferating high-density cell cultures and ideal for supporting T- and B-lymphocytes. It is further noted that Denarase reagent (C-Lecta GmbH) is equivalent to Benzonase reagent in the same quantity in the present process.

In some embodiments, the amount of heparin in the grind media is about 5 U/ml to about 15 U/ml. In some embodiments, the amount of heparin in the grind media is about 5 U/ml to about 6 U/ml, about 5 U/ml to about 7 U/ml, about 5 U/ml to about 8 U/ml, about 5 U/ml to about 9 U/ml, about 5 U/ml to about 10 U/ml, about 5 U/ml to about 11 U/ml, about 5 U/ml to about 12 U/ml, about 5 U/ml to about 13 U/ml, about 5 U/ml to about 14 U/ml, about 5 U/ml to about 15 U/ml, about 6 U/ml to about 7 U/ml, about 6 U/ml to about 8 U/ml, about 6 U/ml to about 9 U/ml, about 6 U/ml to about 10 U/ml, about 6 U/ml to about 11 U/ml, about 6 U/ml to about 12 U/ml, about 6 U/ml to about 13 U/ml, about 6 U/ml to about 14 U/ml, about 6 U/ml to about 15 U/ml, about 7 U/ml to about 8 U/ml, about 7 U/ml to about 9 U/ml, about 7 U/ml to about 10 U/ml, about 7 U/ml to about 11 U/ml, about 7 U/ml to about 12 U/ml, about 7 U/ml to about 13 U/ml, about 7 U/ml to about 14 U/ml, about 7 U/ml to about 15 U/ml, about 8 U/ml to about 9 U/ml, about 8 U/ml to about 10 U/ml, about 8 U/ml to about 11 U/ml, about 8 U/ml to about 12 U/ml, about 8 U/ml to about 13 U/ml, about 8 U/ml to about 14 U/ml, about 8 U/ml to about 15 U/ml, about 9 U/ml to about 10 U/ml, about 9 U/ml to about 11 U/ml, about 9 U/ml to about 12 U/ml, about 9 U/ml to about 13 U/ml, about 9 U/ml to about 14 U/ml, about 9 U/ml to about 15 U/ml, about 10 U/ml to about 11 U/ml, about 10 U/ml to about 12 U/ml, about 10 U/ml to about 13 U/ml, about 10 U/ml to about 14 U/ml, about 10 U/ml to about 15 U/ml, about 11 U/ml to about 12 U/ml, about 11 U/ml to about 13 U/ml, about 11 U/ml to about 14 U/ml, about 11 U/ml to about 15 U/ml, about 12 U/ml to about 13 U/ml, about 12 U/ml to about 14 U/ml, about 12 U/ml to about 15 U/ml, about 13 U/ml to about 14 U/ml, about 13 U/ml to about 15 U/ml, or about 14 U/ml to about 15 U/ml. In some embodiments, the amount of heparin in the grind media is about 5 U/ml, about 6 U/ml, about 7 U/ml, about 8 U/ml, about 9 U/ml, about 10 U/ml, about 11 U/ml, about 12 U/ml, about 13 U/ml, about 14 U/ml, or about 15 U/ml. In some embodiments, the amount of heparin in the grind media is at least about 5 U/ml, about 6 U/ml, about 7 U/ml, about 8 U/ml, about 9 U/ml, about 10 U/ml, about 11 U/ml, about 12 U/ml, about 13 U/ml, or about 14 U/ml. In some embodiments, the amount of heparin in the grind media is at most about 6 U/ml, about 7 U/ml, about 8 U/ml, about 9 U/ml, about 10 U/ml, about 11 U/ml, about 12 U/ml, about 13 U/ml, about 14 U/ml, or about 15 U/ml. In some embodiments, the amount of Benzonase® or Denarase® in the grind media is about 11 U/ml to about 55 U/ml. In some embodiments, the amount of Benzonase in the grind media is about 11 U/ml to about 15 U/ml, about 11 U/ml to about 20 U/ml, about 11 U/ml to about 25 U/ml, about 11 U/ml to about 30 U/ml, about 11 U/ml to about 35 U/ml, about 11 U/ml to about 40 U/ml, about 11 U/ml to about 45 U/ml, about 11 U/ml to about 50 U/ml, about 11 U/ml to about 55 U/ml, about 15 U/ml to about 20 U/ml, about 15 U/ml to about 25 U/ml, about 15 U/ml to about 30 U/ml, about 15 U/ml to about 35 U/ml, about 15 U/ml to about 40 U/ml, about 15 U/ml to about 45 U/ml, about 15 U/ml to about 50 U/ml, about 15 U/ml to about 55 U/ml, about 20 U/ml to about 25 U/ml, about 20 U/ml to about 30 U/ml, about 20 U/ml to about 35 U/ml, about 20 U/ml to about 40 U/ml, about 20 U/ml to about 45 U/ml, about 20 U/ml to about 50 U/ml, about 20 U/ml to about 55 U/ml, about 25 U/ml to about 30 U/ml, about 25 U/ml to about 35 U/ml, about 25 U/ml to about 40 U/ml, about 25 U/ml to about 45 U/ml, about 25 U/ml to about 50 U/ml, about 25 U/ml to about 55 U/ml, about 30 U/ml to about 35 U/ml, about 30 U/ml to about 40 U/ml, about 30 U/ml to about 45 U/ml, about 30 U/ml to about 50 U/ml, about 30 U/ml to about 55 U/ml, about 35 U/ml to about 40 U/ml, about 35 U/ml to about 45 U/ml, about 35 U/ml to about 50 U/ml, about 35 U/ml to about 55 U/ml, about 40 U/ml to about 45 U/ml, about 40 U/ml to about 50 U/ml, about 40 U/ml to about 55 U/ml, about 45 U/ml to about 50 U/ml, about 45 U/ml to about 55 U/ml, or about 50 U/ml to about 55 U/ml. In some embodiments, the amount of Benzonase in the grind media is about 11 U/ml, about 15 U/ml, about 20 U/ml, about 25 U/ml, about 30 U/ml, about 35 U/ml, about 40 U/ml, about 45 U/ml, about 50 U/ml, or about 55 U/ml. In some embodiments, the amount of Benzonase in the grind media is at least about 11 U/ml, about 15 U/ml, about 20 U/ml, about 25 U/ml, about 30 U/ml, about 35 U/ml, about 40 U/ml, about 45 U/ml, or about 50 U/ml. In some embodiments, the amount of Benzonase in the grind media is at most about 15 U/ml, about 20 U/ml, about 25 U/ml, about 30 U/ml, about 35 U/ml, about 40 U/ml, about 45 U/ml, about 50 U/ml, or about 55 U/ml.

In some embodiments, the amount of Benzonase® in the grind media is about 1 U/ml to about 10 U/ml. In some embodiments, the amount of Benzonase in the grind media is about 1 U/ml to about 2 U/ml, about 1 U/ml to about 3 U/ml, about 1 U/ml to about 4 U/ml, about 1 U/ml to about 5 U/ml, about 1 U/ml to about 6 U/ml, about 1 U/ml to about 7 U/ml, about 1 U/ml to about 8 U/ml, about 1 U/ml to about 9 U/ml, about 1 U/ml to about 10 U/ml, about 2 U/ml to about 3 U/ml, about 2 U/ml to about 4 U/ml, about 2 U/ml to about 5 U/ml, about 2 U/ml to about 6 U/ml, about 2 U/ml to about 7 U/ml, about 2 U/ml to about 8 U/ml, about 2 U/ml to about 9 U/ml, about 2 U/ml to about 10 U/ml, about 3 U/ml to about 4 U/ml, about 3 U/ml to about 5 U/ml, about 3 U/ml to about 6 U/ml, about 3 U/ml to about 7 U/ml, about 3 U/ml to about 8 U/ml, about 3 U/ml to about 9 U/ml, about 3 U/ml to about 10 U/ml, about 4 U/ml to about 5 U/ml, about 4 U/ml to about 6 U/ml, about 4 U/ml to about 7 U/ml, about 4 U/ml to about 8 U/ml, about 4 U/ml to about 9 U/ml, about 4 U/ml to about 10 U/ml, about 5 U/ml to about 6 U/ml, about 5 U/ml to about 7 U/ml, about 5 U/ml to about 8 U/ml, about 5 U/ml to about 9 U/ml, about 5 U/ml to about 10 U/ml, about 6 U/ml to about 7 U/ml, about 6 U/ml to about 8 U/ml, about 6 U/ml to about 9 U/ml, about 6 U/ml to about 10 U/ml, about 7 U/ml to about 8 U/ml, about 7 U/ml to about 9 U/ml, about 7 U/ml to about 10 U/ml, about 8 U/ml to about 9 U/ml, about 8 U/ml to about 10 U/ml, or about 9 U/ml to about 10 U/ml. In some embodiments, the amount of Benzonase in the grind media is about 1 U/ml, about 2 U/ml, about 3 U/ml, about 4 U/ml, about 5 U/ml, about 6 U/ml, about 7 U/ml, about 8 U/ml, about 9 U/ml, or about 10 U/ml. In some embodiments, the amount of Benzonase in the grind media is at least about 1 U/ml, about 2 U/ml, about 3 U/ml, about 4 U/ml, about 5 U/ml, about 6 U/ml, about 7 U/ml, about 8 U/ml, or about 9 U/ml. In some embodiments, the amount of Benzonase in the grind media is at most about 2 U/ml, about 3 U/ml, about 4 U/ml, about 5 U/ml, about 6 U/ml, about 7 U/ml, about 8 U/ml, about 9 U/ml, or about 10 U/ml.

In some embodiments, HSA is present in the grind media at about 0.5% to about 5%. In some embodiments, HSA is present in the grind media at about 0.5% to about 1%, about 0.5% to about 1.5%, about 0.5% to about 2%, about 0.5% to about 2.5%, about 0.5% to about 3%, about 0.5% to about 3.5%, about 0.5% to about 4%, about 0.5% to about 4.5%, about 0.5% to about 5%, about 1% to about 1.5%, about 1% to about 2%, about 1% to about 2.5%, about 1% to about 3%, about 1% to about 3.5%, about 1% to about 4%, about 1% to about 4.5%, about 1% to about 5%, about 1.5% to about 2%, about 1.5% to about 2.5%, about 1.5% to about 3%, about 1.5% to about 3.5%, about 1.5% to about 4%, about 1.5% to about 4.5%, about 1.5% to about 5%, about 2% to about 2.5%, about 2% to about 3%, about 2% to about 3.5%, about 2% to about 4%, about 2% to about 4.5%, about 2% to about 5%, about 2.5% to about 3%, about 2.5% to about 3.5%, about 2.5% to about 4%, about 2.5% to about 4.5%, about 2.5% to about 5%, about 3% to about 3.5%, about 3% to about 4%, about 3% to about 4.5%, about 3% to about 5%, about 3.5% to about 4%, about 3.5% to about 4.5%, about 3.5% to about 5%, about 4% to about 4.5%, about 4% to about 5%, or about 4.5% to about 5%. In some embodiments, HSA is present in the grind media at about 0.5%, about 1%, about 1.5%, about 2%, about 2.5%, about 3%, about 3.5%, about 4%, about 4.5%, or about 5%. In some embodiments, HSA is present in the grind media at least about 0.5%, about 1%, about 1.5%, about 2%, about 2.5%, about 3%, about 3.5%, about 4% or about 4.5%. In some embodiments, HSA is present in the grind media at most about 1% about 1.5%, about 2%, about 2.5%, about 3%, about 3.5%, about 4%, about 4.5%, or about 5%.

Another pitcher of 300-500 ml of grind media is retained for collecting the bone fragments after grinding, and another supply of about 100 ml of the grind media is retained for rinsing through the grinder during the grinding process to prevent bone fragments from sticking to the surface of the pitcher of the grinding components. In some embodiments, the additional grind media may have different quantities of heparin, HSA, and Benzonase as compared to the initial grind media.

An electric bone grinder or a purpose-built bone grinder, such as the grinder of Biorep Technologies Inc, (Miami, FL) can be used in an ISO-5 environment within an ISO-7 clean room. Bone types are kept separate if both VB and ilium from the same donor are being processed. The bone is kept submerged in grind media at all times during and after the grinding process. Once all of the donor bone pieces are ground, the chamber of the bone grinder is thoroughly rinsed with fresh processing media. The bone fragments are discharged from the grinder into the pitcher containing grind media.

The contents of the pitcher are transferred to sterile bags. Next, the contents of the sterile bags are filtered to extract the solid components. In one embodiment, the contents of each bag are passed through a series of stainless-steel sieves. In this embodiment, a No. 40 (425 μm) sieve is stacked on top of a No. 80 (177 μm) sieve, which is seated over a catch-pan to receive the liquid filter contents. The sterile bags containing the output from the grinder is swirled and then poured evenly over the sieve stack or filtration sets. The filtering process is observed to ensure that excessive clumping is not occurring, which can signal the presence of soft tissue or other contaminants. Bone fragments retained on the surface of the sieves are distributed evenly on the sieves and rinsed with 250 ml of fresh processing medium. In one embodiment, the processing medium used for rinsing is the grind media described above or PLASMA-LYTE™ with 2.5% HSA. The sieved bone marrow product, which can be approximately 1000 ml in a well-performed process, is transferred to sterile packs for subsequent processing and analysis. The contents of each bag are visually inspected to confirm that the contents do not include any visible bone fragments or soft tissue.

In some embodiments, the rinse media can contain the various amounts of HSA as described for the grind media. In some embodiments, the rinse media can contain, additionally, heparin and/or Benzonase.

In another embodiment, the contents of each bag are passed through bone marrow filtration units. In this embodiment, the system includes a stand configured to support a sterile collection bag which contains the bone fragments and media from the grinding operation described above. The stand includes a container hanger configured to engage the cap of the sterile bag to suspend the container. The bottom of the bag includes a discharge assembly that includes a pre-filter projecting into the body of the collection bag. In one specific embodiment the pre-filter is an 850 μm filter. In some embodiments, the bone marrow passes first through an 800 μm pre-filter. The filter is connected to an output tube that is connected by a container claim to the input line of a first in-line filter. In the specific embodiment, the first in-line filter is a 200 μm or a 500 μm filter. The output line of the first in-line filter is connected to the input line of a second in-line filter. The second in-line filter is a 200 μm or a 500 μm filter. The two in-line filters are initially both 500 μm for a first pass through the filter system. A second rinse is then performed on the grindings with the two in-line filters being 200 μm. This double-pass filtration results in a cleaner suspension and enhances removal of fat from the suspension. The second in-line filter has an output line that can be engaged to a sterile bag, such as bag for the second filtration pass. On the second pass through the system, the output line of the second in-line filter can be engaged to a container clamp of a transfer pack container. The transfer pack container can be a 600-2000 ml bag to accommodate the filtered bone marrow product, which can be approximately 1000 ml in a well-performed process.

Agitation of Bone Grindings and/or Bone Grinding Filtrate

Described herein, in some embodiments, is a method for processing bone marrow or derivatives thereof, the method comprises mechanically agitating the bone grindings and/or bone grinding filtrate during the grinding and filtration portion of the processing of the bone marrow. In some instances, the bone marrow can be obtained from a deceased donor. In some cases, the bone marrow can be obtained from a sample (e.g. bone or VB) that was previously chilled. In some cases, the bone marrow can be obtained from a sample (e.g. bone or VB) that was previously chilled but not frozen. In some cases, the bone marrow can be obtained from a sample (e.g. bone or VB) that is thawed. In some cases, the bone marrow can be processed for obtaining bone marrow cells. In some embodiments, the bone marrow cells can be hematopoietic stem cells (HSCs). In some embodiments, the bone marrow cells can be mesenchymal stem cells (MSCs).

Aspect disclosed in the present disclosure comprises a method for processing bone marrow or a derivative thereof, wherein the bone marrow or the derivative thereof is derived from a deceased donor, the method comprising: obtaining a bone or bone fragment from a deceased donor, optionally, processing the bone into bone fragments; mechanically grinding the bone or bone fragment in the presence of a grinding solution to generate a plurality of bone grindings; placing the plurality of bone grindings on a shaker at about 100 to about 200 rounds per minute (“RPM”) for about 1 to about 20 minutes; and removing the solution from the shaker, wherein the solution comprises the bone marrow or the derivative thereof and wherein the bone marrow or the derivative thereof comprises at least about 1,500,000 CD34+ cells/ml of the bone marrow or the derivative thereof. In some embodiments, the method further comprises contacting the solution with a rinse media and repeating the placing of the bone grindings on the shaker and then removing the solution from the shaker. In some embodiments, the method further comprises repeating step placing the bone grinding on the shaker and then removing the solution from the shaker one or more times. In some embodiments, the at least about 1,500,000 CD34+ cells/ml of the bone marrow or the derivative thereof comprises at least 85% viable CD34+ cells. In some embodiments, the method further comprises the at least about 1,500,000 CD34+ cells/ml of the bone marrow or the derivative thereof comprises at least 90% viable CD34+ cells.

The mechanical agitation can comprise agitating the bone grindings in a linear fashion. In some embodiments, the mechanical agitation can comprise agitating the bone grindings in a three-dimensional fashion. In some cases, the mechanical agitation of the bone grindings can comprise orbital shaking (via an orbital shaker) such as placing the bone grinding on a shaker. In some cases, the bone grindings can be mechanically agitated by the shaker at a rate at least about 10 rounds per minute (RPM), 20 RPM, 30 RPM, 40 RPM, 50 RPM, 60 RPM, 70 RPM, 80 RPM, 90 RPM, 100 RPM, 110 RPM, 120 RPM, 130 RPM, 140 RPM, 150 RPM, 160 RPM, 170 RPM, 180 RPM, 190 RPM, 200 RPM, 210 RPM, 220 RPM, 230 RPM, 240 RPM, 250 RPM, or more. In some cases, the bone grindings can be mechanically agitated by centrifugation (e.g. spinning). In some embodiments, the bone grindings can be spun at least 10 RPM, 20 RPM, 30 RPM, 40 RPM, 50 RPM, 60 RPM, 70 RPM, 80 RPM, 90 RPM, 100 RPM, 110 RPM, 120 RPM, 130 RPM, 140 RPM, 150 RPM, 160 RPM, 170 RPM, 180 RPM, 190 RPM, 200 RPM, 210 RPM, 220 RPM, 230 RPM, 240 RPM, 250 RPM, or more. In some embodiments, the bone grindings can be spun at least 300 RPM, 400 RPM, 500 RPM, 600 RPM, or more. In some embodiments, the bone grindings can be mechanically agitated by both shaking and spinning. In some embodiments, the mechanical agitation of the bone grindings can be for at least 1 minute, 2 minutes, 3 minutes, 4 minutes, 5 minutes, 10 minutes, 15 minutes, 20 minutes, 25 minutes, 30 minutes, or longer.

In some embodiments, the mechanical agitation of the bone grindings increases the yield of the bone marrow cells obtained. In some instances, the yield of the bone marrow cells obtained by mechanical agitation of the bone grindings is increased by at least about 10%, 20%, 30%, 40%, 50%, 60%, 70%, 80%. 90%, 100%, 2-fold, 3-fold, 4-fold, 5-fold, 10-fold, 20-fold, 50-fold, or more compared to yield of bone marrow cells obtained without the mechanical agitation.

In some embodiments, the mechanical agitation of the bone grindings increases the viability of the bone marrow cells obtained. In some instances, the viability of the bone marrow cells obtained by mechanical agitation of the bone grindings is increased by at least about 10%, 20%, 30%, 40%, 50%, 60%, 70%, 80%. 90%, 100%, 2-fold, 3-fold, 4-fold, 5-fold, 10-fold, 20-fold, 50-fold, or more compared to the viability of bone marrow cells obtained without the mechanical agitation.

In some embodiments, the mechanical agitation of the bone grindings increases the number of CD34 expressing bone marrow cells obtained. In some instances, the number of CD34 expressing bone marrow cells obtained by mechanical agitation of the bone grindings is increased by at least about 10%, 20%, 30%, 40%, 50%, 60%, 70%, 80%. 90%, 100%, 2-fold, 3-fold, 4-fold, 5-fold, 10-fold, 20-fold, 50-fold, or more compared to the number of CD34 expressing bone marrow cells obtained without the mechanical agitation.

In some embodiments, the mechanical agitation of the bone grindings increases the number of CD45 expressing bone marrow cells obtained by the methods described herein. In some instances, the number of CD45 expressing bone marrow cells obtained by mechanical agitation of the bone grindings is increased by at least about 10%, 20%, 30%, 40%, 50%, 60%, 70%, 80%. 90%, 100%, 2-fold, 3-fold, 4-fold, 5-fold, 10-fold, 20-fold, 50-fold, or more compared to the number of CD45 expressing bone marrow cells obtained without the mechanical agitation.

In some embodiments, the amount of CD34+ cells/ml of the bone marrow or the derivative thereof obtained is at least about 1,500,000 CD34+ cells/ml to about 2,000,000 CD34+ cells/ml. In some embodiments, the amount of CD34+ cells/ml of the bone marrow or the derivative thereof obtained is at least about 1,500,000 CD34+ cells/ml to about 1,750,000 CD34+ cells/ml, about 1,500,000 CD34+ cells/ml to about 2,000,000 CD34+ cells/ml, or about 1,750,000 CD34+ cells/ml to about 2,000,000 CD34+ cells/ml. In some embodiments, the amount of CD34+ cells/ml of the bone marrow or the derivative thereof obtained is at least about 1,500,000 CD34+ cells/ml, about 1,750,000 CD34+ cells/ml, or about 2,000,000 CD34+ cells/ml. In some embodiments, the amount of CD34+ cells/ml of the bone marrow or the derivative thereof obtained is at least at least about 1,500,000 CD34+ cells/ml, or about 1,750,000 CD34+ cells/ml. In some embodiments, the amount of CD34+ cells/ml of the bone marrow or the derivative thereof obtained is at least at most about 1,750,000 CD34+ cells/ml, or about 2,000,000 CD34+ cells/ml.

In some embodiments, the viability of the CD34+ cells is at least about 70% to about 95%.

In some embodiments, the viability of the CD34+ cells is at least about 70% to about 75%, about 70% to about 80%, about 70% to about 85%, about 70% to about 90%, about 70% to about 95%, about 75% to about 80%, about 75% to about 85%, about 75% to about 90%, about 75% to about 95%, about 80% to about 85%, about 80% to about 90%, about 80% to about 95%, about 85% to about 90%, about 85% to about 95%, or about 90% to about 95%. In some embodiments, the viability of the CD34+ cells is at least about 70%, about 75%, about 80%, about 85%, about 90%, or about 95%. In some embodiments, the viability of the CD34+ cells is at least at least about 70%, about 75%, about 80%, about 85%, or about 90%. In some embodiments, the viability of the CD34+ cells is at least at most about 75%, about 80%, about 85%, about 90%, or about 95%.

For quality control, a small quantity of bone marrow, such as 0.3 mL, is extracted from the sterile pack using a syringe at an injection site and conducting inversion mixing before pulling the sample. The sample can be tested by a hematology analyzer, such as a Sysmex Hematology Analyzer, to determine the total nucleated cell (TNC) content of the sample, as an indicator of the TNC content of the bone marrow being subsequently processed.

### Fat Removal and Concentration

The bone marrow product collected from the filtering is essentially a fatty emulsion. The fat content of the suspension obtained from the sieve filtering approach disclosed above is greater than the fat content of the suspension obtained from the double-pass filtration system. However, in both cases, there is a need to remove the fat content from the suspension. The suspension obtained from the filtering is recovered into 250 ml bags which are hermetically sealed with tube welders. Pairs of sterile bags and taring sticks are mounted within a centrifuge with bag ports facing down, and balanced. Volume compensating plates are used to prevent creasing of the bags during centrifugation. In one embodiment, the bags are centrifuged at 500×g for 15 minutes at room temperature to concentrate the cells, preferably to 2-3×10⁸/ml. After centrifugation is complete, each bag is individually hung on a ring stand.

The distinct layers within the bag are visible, with the fat layer clearly delineated on top of the supernatant with the bone marrow pellet at the bottom. A new sterile bag is welded to the bag removed from the centrifuge. A bag clamp or clip is placed on the bag just below the fat layer, to clamp off or squeeze the bag closed beneath the fat layer. The pellet is then drained from the centrifuge bag into the new sterile bag, with the bag clip preventing passage of the fat layer. The pellet is agitated as it is drained to resuspend all of the pellet. After about half of the pellet has drained into the new bag, the tubing is closed with a hemostat or tube sealer. The second centrifuge bag is then welded to the new bag containing the pellet, and the contents of this second centrifuge bag are drained into the new bag.

The result is new sterile bags containing the bone marrow centrifuged to remove the fat. These bags of de-fatted bone marrow are then centrifuged at 500×g for 15 minutes at room temperature, with volume compensating plates to prevent creasing of the bags. Each bag is removed and suspended on a ring stand and a waste bag is welded to the bag, and a plasma extractor is used to remove the supernatant into the waste bag. The tubing is clamped with a hemostat when the pellet rises or breaks. The tubing is then sealed and severed to remove the pellet-containing bag from the waste bag, which is discarded. A Luer connection is welded to the pellet-containing bag. The pellets from each bag are combined into a bulk bag using a large syringe. The pellet-containing bags are rinsed into the bulk bag using a rinse media. The bulk bag is inverted several times to ensure that all of the pellet is resuspended. A quantity of the processed BM, such as 0.5 mL, can be removed for quality control testing for density and cell count. The test sample can also be evaluated for human leukocyte antigens, CCR5 delta 32 mutation and apolipoprotein (APOE), among other things.

In some embodiments, the centrifuge settings at one or more steps can be increased. In some embodiments, the centrifuge is spun at about 400 g to about 650 g. In some embodiments, the centrifuge is spun at about 400 g to about 450 g, about 400 g to about 500 g, about 400 g to about 550 g, about 400 g to about 600 g, about 400 g to about 650 g, about 450 g to about 500 g, about 450 g to about 550 g, about 450 g to about 600 g, about 450 g to about 650 g, about 500 g to about 550 g, about 500 g to about 600 g, about 500 g to about 650 g, about 550 g to about 600 g, about 550 g to about 650 g, or about 600 g to about 650 g. In some embodiments, the centrifuge is spun at about 400 g, about 450 g, about 500 g, about 550 g, about 600 g, or about 650 g. In some embodiments, the centrifuge is spun at least about 400 g, about 450 g, about 500 g, about 550 g, or about 600 g. In some embodiments, the centrifuge is spun at most about 450 g, about 500 g, about 550 g, about 600 g, or about 650 g. In some embodiments, the centrifuge is spun for about 10 minutes to about 40 minutes. In some embodiments, the centrifuge is spun for about 10 minutes to about 15 minutes, about 10 minutes to about 20 minutes, about 10 minutes to about 25 minutes, about 10 minutes to about 30 minutes, about 10 minutes to about 35 minutes, about 10 minutes to about 40 minutes, about 15 minutes to about 20 minutes, about 15 minutes to about 25 minutes, about 15 minutes to about 30 minutes, about 15 minutes to about 35 minutes, about 15 minutes to about 40 minutes, about 20 minutes to about 25 minutes, about 20 minutes to about 30 minutes, about 20 minutes to about 35 minutes, about 20 minutes to about 40 minutes, about 25 minutes to about 30 minutes, about 25 minutes to about 35 minutes, about 25 minutes to about 40 minutes, about 30 minutes to about 35 minutes, about 30 minutes to about 40 minutes, or about 35 minutes to about 40 minutes. In some embodiments, the centrifuge is spun for about 10 minutes, about 15 minutes, about 20 minutes, about 25 minutes, about 30 minutes, about 35 minutes, or about 40 minutes. In some embodiments, the centrifuge is spun for at least about 10 minutes, about 15 minutes, about 20 minutes, about 25 minutes, about 30 minutes, or about 35 minutes. In some embodiments, the centrifuge is spun for at most about 15 minutes, about 20 minutes, about 25 minutes, about 30 minutes, about 35 minutes, or about 40 minutes. In some embodiments, the centrifuge is stopped without the use of a brake. In some embodiments, the centrifuge is stopped with a brake. In some embodiments, the centrifuge brake is set at about 25% to about 100%. In some embodiments, the centrifuge brake is set at about 25% to about 50%, about 25% to about 75%, about 25% to about 100%, about 50% to about 75%, about 50% to about 100%, or about 75% to about 100%. In some embodiments, the centrifuge brake is set at about 25%, about 50%, about 75%, or about 100%. In some embodiments, the centrifuge brake is set at least about 25%, about 50%, or about 75%. In some embodiments, the centrifuge brake is set at most about 50%, about 75%, or about 100%.

### Isolation of CD34+ Cells

Described herein, in some aspects, is a method for processing (e.g. isolating) CD34+ cells obtained from bone marrow or bone marrow derivative. In some cases, the bone marrow or bone marrow derivative can be fresh (e.g. never frozen) or thawed from being previously frozen. In some embodiments, the bone marrow or bone marrow derivative can be ground by the methods and systems described herein. In some embodiments, ground bone marrow or bone marrow cells can be contacted with the stabilization buffer described herein. In some embodiments, the stabilization prevents formation of aggregates of the bone marrow cells. In some instances, the bone marrow cells contacted and suspended in the stabilization buffer can be isolated by attaching to antibody such as a conjugated antibody. For example, bone marrow cells expressing CD34+ can be isolated and enriched by contacting the bone marrow cells with the CD34 antibody conjugated with iron, where the bone marrow cells expressing CD34 are then trapped a magnetic separation column (e.g. “CliniMACS®”). The bone marrow cells not expressing CD34 are can be washed away. The trapped CD34+ bone marrow cells can be harvested by removing the magnetic field and eluting the targeted CD34+ bone marrow cells. Such approach does not require isolating the bone marrow cells with a Ficoll gradient.

Aspect described in the present disclosure comprises a method for processing a population of CD34+ cells obtained from bone marrow or a derivative thereof, wherein the bone marrow or the derivative thereof is derived from a deceased donor, the method comprising: obtaining a bone or bone fragment from a deceased donor, optionally, processing the bone into bone fragments; extracting the bone marrow or derivative thereof from the bone or bone fragment; and contacting the bone marrow or derivative thereof with a stabilization buffer, wherein the stabilization buffer comprises more than about 3 U/ml of a nuclease; performing a CD34+ cell isolation assay to generate a cellular composition comprising the population of CD34+ cells, wherein the composition comprising the population of CD34+ cells comprises at least about 80,000 CD34+ cells/750 μl of the bone marrow or the derivative thereof contacted with the stabilization buffer. In some embodiments, the at least about 80,000 CD34+ cells/750 μl of the bone marrow or the derivative thereof contacted with the stabilization buffer comprise at least 70% viable CD34+ cells. In some embodiments, the at least about 80,000 CD34+ cells/750 μl of the bone marrow or the derivative thereof contacted with the stabilization buffer comprise at least 80% viable CD34+ cells. In some embodiments, the at least about 80,000 CD34+ cells/750 μl of the bone marrow or the derivative thereof contacted with the stabilization buffer comprise at least 90% viable CD34+ cells.

Another aspect of the present disclosure comprises a stabilization buffer comprising: at least 5 U/ml of an anticoagulant; and more than 3 U/ml of a nuclease. In some embodiments, stabilization buffer comprises more than about 5 U/ml of a nuclease. In some embodiments, the stabilization buffer comprises more than about 10 U/ml of a nuclease. In some embodiments, the stabilization buffer comprises more than about 15 U/ml of a nuclease. In some embodiments, the stabilization buffer comprises more than about 20 U/ml of a nuclease. In some embodiments, the stabilization buffer comprises about 20 U/ml of a nuclease. In some embodiments, the nuclease is Benzonase® or Denarase®. In some embodiments, the stabilization buffer further comprises more than about 10 U/ml of an anticoagulant. In some embodiments, the stabilization buffer further comprises about 10 U/ml of an anticoagulant. In some embodiments, the anticoagulant is heparin. In some embodiments, the stabilization buffer further comprises human serum albumin (HSA). In some embodiments, the stabilization buffer comprises 0.5% HSA.

In some embodiments, the stabilization buffer comprises nuclease. In some embodiments, the nuclease is Benzonase® or Denarase®. In some embodiments, the stabilization buffer comprises nuclease at about 3 U/ml, 4 U/ml, 5 U/ml, 6 U/ml, 7 U/ml, 8 U/ml, 9 U/ml, 10 U/ml, 11 U/ml, 12 U/ml, 13 U/ml, 14 U/ml, 15 U/ml, 16 U/ml, 17 U/ml, 18 U/ml, 19 U/ml, 20 U/ml, 21 U/ml, 22 U/ml, 23 U/ml, 24 U/ml, 25 U/ml, 26 U/ml, 27 U/ml, 28 U/ml, 29 U/ml, 30 U/ml, 50 U/ml, 100 U/ml, 200 U/ml, or more U/ml. In some embodiments, the stabilization buffer comprises an anticoagulant. In some cases, the anticoagulant is Heparin.

In some instances, the stabilization buffer comprises anticoagulant at about 0.1 U/ml, 0.2 U/ml, 0.3 U/ml, 0.4 U/ml, 0.5 U/ml, 0.6 U/ml, 0.7 U/ml, 0.8 U/ml, 0.9 U/ml, 1.0 U/ml, 2.0 U/ml, 3.0 U/ml, 4.0 U/ml, 5.0 U/ml, 6.0 U/ml, 7.0 U/ml, 8.0 U/ml, 9.0 U/ml, 10 U/ml, 11 U/ml, 12 U/ml, 13 U/ml, 14 U/ml, 15 U/ml, 16 U/ml, 17 U/ml, 18 U/ml, 19 U/ml, 20 U/ml, 21 U/ml, 22 U/ml, 23 U/ml, 24 U/ml, 25 U/ml, 26 U/ml, 27 U/ml, 28 U/ml, 29 U/ml, 30 U/ml, 50 U/ml, 100 U/ml, 200 U/ml, or more U/ml.

In some embodiments, the stabilization buffer comprises about 0.001%, 0.002%, 0.003%, 0.004%, 0.005%, 0.01%, 0.02%, 0.03%, 0.04%, 0.05% HSA, 0.1% HSA, 0.2% HSA, 0.3% HSA, 0.4% HSA, 0.5% HSA, 0.6% HSA, 0.7% HSA, 0.8% HSA, 0.9% HSA, 1.0% HSA, 1.5% HSA, 2% HSA, 2.5% HSA, 5% HSA, 10% HSA, 20% HSA, or more HSA.

Described herein, in some embodiments, is a method of processing bone marrow to obtain bone marrow cells. In some embodiments, the method comprises contacting the bone marrow or the bone marrow cells with the stabilization buffer described herein.

Another aspect of the present disclosure comprises a method for processing a population of CD34+ cells comprised in bone marrow or a derivative thereof, wherein the bone marrow or the derivative thereof is derived from a deceased donor, the method comprising: obtaining a bone or bone fragment from a deceased donor, optionally, processing the bone into bone fragments; extracting the bone marrow or derivative thereof from the bone or bone fragment; and contacting the bone marrow or derivative thereof with a stabilization buffer, wherein the stabilization buffer comprises more than about 3 U/ml of a nuclease; performing a CD34+ cell isolation assay to generate a cellular composition comprising the population of CD34+ cells, wherein the composition comprising the population of CD34+ cells comprises at least about 80,000 CD34+ cells/750 ul of the bone marrow or the derivative thereof contacted with the stabilization buffer.

In some embodiments, processing or contacting the bone marrow or bone marrow cells described herein with the stabilization buffer increases the yield of the bone marrow cells obtained from the methods described herein compared to the yield of the bone marrow cells processed in the absence of the stabilization buffer. In some instances, processing or contacting the bone marrow or bone marrow cells described herein with the stabilization buffer increases the yield of the bone marrow cells by at least about 10%, 20%, 30%, 40%, 50%, 60%, 70%, 80%. 90%, 100%, 2-fold, 3-fold, 4-fold, 5-fold, 10-fold, 20-fold, 50-fold, or more compared to yield of bone marrow cells processed in the absence of the stabilization buffer. In some embodiments, processing or contacting the bone marrow or bone marrow cells described herein with the stabilization buffer increases the viability of the bone marrow cells obtained from the methods described herein compared to the viability of the bone marrow cells processed in the absence of the stabilization buffer. In some instances, processing or contacting the bone marrow or bone marrow cells described herein with the stabilization buffer increases the viability of the bone marrow cells by at least about 10%, 20%, 30%, 40%, 50%, 60%, 70%, 80%. 90%, 100%, 2-fold, 3-fold, 4-fold, 5-fold, 10-fold, 20-fold, 50-fold, or more compared to viability of bone marrow cells processed in the absence of the stabilization buffer.

In some embodiments, processing or contacting the bone marrow or bone marrow cells described herein with the stabilization buffer increases the number of CD34+ bone marrow cells compared to the number of CD34+ bone marrow cells processed in the absence of the stabilization buffer. In some cases, the number of CD34+ bone marrow obtained from processing with the stabilization buffer is increased by at least about 10%, 20%, 30%, 40%, 50%, 60%, 70%, 80%. 90%, 100%, 2-fold, 3-fold, 4-fold, 5-fold, 10-fold, 20-fold, 50-fold, or more compared to the number of CD34+ bone marrow obtained from processing in the absence of stabilization buffer. In some embodiments, processing or contacting the bone marrow or bone marrow cells described herein with the stabilization buffer increases the number of CD45+ bone marrow cells compare to the number of CD45+ bone marrow cells processed in the absence of the stabilization buffer. In some cases, the number of CD45+ bone marrow obtained from processing with the stabilization buffer is increased by at least about 10%, 20%, 30%, 40%, 50%, 60%, 70%, 80%. 90%, 100%, 2-fold, 3-fold, 4-fold, 5-fold, 10-fold, 20-fold, 50-fold, or more compared to the number of CD45+ bone marrow obtained from processing in the absence of stabilization buffer.

In some embodiments, cellular compositions comprising CD34+ cells derived from bone marrow samples processed with the stabilization buffers described herein have an increased amount of CD34+ cells, as compared to cellular compositions generated from known CD34+ isolation methods. In some embodiments. The amount of CD34+ cells isolated from the bone marrow samples contacted with the stabilization buffers described herein is at least about 70,000 CD34+ cells/750 ul of bone marrow or a derivative thereof contacted with the stabilization buffers described herein. In some embodiments, the amount of CD34+ cells isolated from the bone marrow samples contacted with the stabilization buffers described herein is at least about 70,000 cells/750 ul to about 100,000 cells/750 ul. In some embodiments, the amount of CD34+ cells isolated from the bone marrow samples contacted with the stabilization buffers described herein is at least about 70,000 cells/750 ul to about 75,000 cells/750 ul, about 70,000 cells/750 ul to about 80,000 cells/750 ul, about 70,000 cells/750 ul to about 85,000 cells/750 ul, about 70,000 cells/750 ul to about 90,000 cells/750 ul, about 70,000 cells/750 ul to about 95,000 cells/750 ul, about 70,000 cells/750 ul to about 100,000 cells/750 ul, about 75,000 cells/750 ul to about 80,000 cells/750 ul, about 75,000 cells/750 ul to about 85,000 cells/750 ul, about 75,000 cells/750 ul to about 90,000 cells/750 ul, about 75,000 cells/750 ul to about 95,000 cells/750 ul, about 75,000 cells/750 ul to about 100,000 cells/750 ul, about 80,000 cells/750 ul to about 85,000 cells/750 ul, about 80,000 cells/750 ul to about 90,000 cells/750 ul, about 80,000 cells/750 ul to about 95,000 cells/750 ul, about 80,000 cells/750 ul to about 100,000 cells/750 ul, about 85,000 cells/750 ul to about 90,000 cells/750 ul, about 85,000 cells/750 ul to about 95,000 cells/750 ul, about 85,000 cells/750 ul to about 100,000 cells/750 ul, about 90,000 cells/750 ul to about 95,000 cells/750 ul, about 90,000 cells/750 ul to about 100,000 cells/750 ul, or about 95,000 cells/750 ul to about 100,000 cells/750 ul. In some embodiments, the amount of CD34+ cells isolated from the bone marrow samples contacted with the stabilization buffers described herein is at least about 70,000 cells/750 ul, about 75,000 cells/750 ul, about 80,000 cells/750 ul, about 85,000 cells/750 ul, about 90,000 cells/750 ul, about 95,000 cells/750 ul, or about 100,000 cells/750 ul. In some embodiments, the amount of CD34+ cells isolated from the bone marrow samples contacted with the stabilization buffers described herein is at least at least about 70,000 cells/750 ul, about 75,000 cells/750 ul, about 80,000 cells/750 ul, about 85,000 cells/750 ul, about 90,000 cells/750 ul, or about 95,000 cells/750 ul. In some embodiments, the amount of CD34+ cells isolated from the bone marrow samples contacted with the stabilization buffers described herein is at least at most about 75,000 cells/750 ul, about 80,000 cells/750 ul, about 85,000 cells/750 ul, about 90,000 cells/750 ul, about 95,000 cells/750 ul, or about 100,000 cells/750 ul.

In some embodiments, the CD34+ cells derived from bone marrow samples processed with the stabilization buffers described herein also exhibit higher viability as compared to cellular compositions generated from known CD34+ isolation methods.

In some embodiments, the amount of CD34+ cells isolated from the bone marrow samples contacted with the stabilization buffers described herein comprise a percent viability of at least about 70% to about 95%. In some embodiments, the amount of CD34+ cells isolated from the bone marrow samples contacted with the stabilization buffers described herein comprise a percent viability of at least about 70% to about 95%. In some embodiments, the amount of CD34+ cells isolated from the bone marrow samples contacted with the stabilization buffers described herein comprise a percent viability of at least about 70% to about 75%, about 70% to about 80%, about 70% to about 85%, about 70% to about 90%, about 70% to about 95%, about 75% to about 80%, about 75% to about 85%, about 75% to about 90%, about 75% to about 95%, about 80% to about 85%, about 80% to about 90%, about 80% to about 95%, about 85% to about 90%, about 85% to about 95%, or about 90% to about 95%. In some embodiments, the amount of CD34+ cells isolated from the bone marrow samples contacted with the stabilization buffers described herein comprise a percent viability of at least about 70%, about 75%, about 80%, about 85%, about 90%, or about 95%. In some embodiments, the amount of CD34+ cells isolated from the bone marrow samples contacted with the stabilization buffers described herein comprise a percent viability of at least at least about 70%, about 75%, about 80%, about 85%, or about 90%. In some embodiments, the amount of CD34+ cells isolated from the bone marrow samples contacted with the stabilization buffers described herein comprise a percent viability of at least at most about 75%, about 80%, about 85%, about 90%, or about 95%.

In an aspect of the present disclosure, a method is provided for selecting CD34 expressing (CD34+) cells from deceased donor bone marrow using density reduced Ficoll and an immunomagnetic CD34+ cell isolation kit. Surprisingly, it has been found that cell isolation using density reduced Ficoll prior to CD34 selection is beneficial to obtain high purity and viability CD45/CD34+ cells from freshly prepared deceased donor bone marrow. On the other hand, Ficoll at conventional density has been found to be optimal for CD45/CD34+ cell selection from thawed cryopreserved deceased donor bone marrow.

Vertebral sections obtained from a recently deceased donor were processed as described above. Thus, in one embodiment, the bone is cleaned of all soft tissue and then cut into small pieces that were immediately submerged into 500 ml of grinding media. The grinding media can be PLASMA-LYTE™ A injection pH 7.4, multiple electrolytes, injection type 1 USP (PLASMA-LYTE™) containing 2.5% human serum albumin (HSA), 3 U/ml Denarase, and 10 U/ml heparin. The sectioned VB are ground using a bone grinder, filtered and rinsed with rinse media (such as PLASMA-LYTE™ with 2.5% HSA). The entire cell suspension is centrifuged to concentrate cells to 2-3×10⁸/ml and the cell concentration is extracted. A portion or all of the resulting BM preparation can be used immediately for CD34 selection, while the remainder can be prepared for cryopreservation. The cryopreserved portion involves adding a final concentration of 10% DMSO and 5% HSA to the BM cells and bringing the preparation to −86° C., either by passive cooling or by controlled cooling at a rate of approximately −1° C./min, after which the cryopreserved portion is plunged into liquid nitrogen.

For selection of CD34+ cells, either the newly processed BM preparation is used or a previously cryopreserved portion is thawed for use. Ficoll-Paque PLUS is added to the BM preparation to separate the desired CD34+ cell component of the bone marrow. It has been found for cell selection from cryopreserved bone marrow that the conventional density for the Ficoll of 1.077 g/ml produces acceptable results. However, in one aspect of the present disclosure, for cell selection from freshly prepared deceased donor bone marrow the Ficoll density is reduced from the conventional density. In particular, the density is reduced by mixing Ficoll-Paque PLUS (density 1.077 g/ml, GE Company) with Plasma Lyte-A Injection pH 7.4 (Baxter Healthcare 2B2544X) in specific proportions to obtain an overall density of less than 1.077 g/ml, particularly 1.063-1.052 g/ml. In one specific embodiment, the density of 1.063 g/ml was found to be optimal for isolation of CD34+ cells, taking into account quantity, viability and purity of the CD34+ cells.

In one embodiment, 5 ml of the 1.063 g/ml density Ficoll solutions is pipetted into 15-ml centrifuge tubes, and the BM solution generated from VBs of deceased donors is carefully layered over the Ficoll gradient. The tubes are centrifuged for 30 min at 400 g without break at room temperature. After centrifugation, buffy coat cells are harvested carefully, and the cells are washed in phosphate-buffered saline (PBS) containing 0.5% HSA and 2 mM Ethylenediaminetetraacetic acid (EDTA) (MACS buffer, Miltenyi). In one specific embodiment, centrifugation is performed for 5 min at 400 g, and the resulting cell pellets are resuspended in 10 ml PBS, followed by a second centrifugation for 5 min at 400 g.

Nucleated cells in the isolated buffy coat can be counted using a Sysmex XP-300. A Cellometer Vision (Nexcellom) or flow cytometer can be used to determine cell counts of purified CD34 cells. 20 microliters of AOPI can be added to 20 microliters of cells and after mixing total viable cells can be determined. The CD34+ cells can be selected by a positive immune separation method using a CliniMAX system (Miltenyi, Bergisch Gladbach, Germany) or an EasySep CD34 kit (Stemcell Technologies, Vancouver, BC, Canada) in accordance with the protocol of the manufacturer. From testing at various Ficoll densities it has been surprisingly determined that the lower Ficoll density contemplated in the present disclosure (i.e., 1.063-1.052 gm/ml vs. the conventional 1.077 gm/ml density) leads to more optimum cell recovery. Optimization is based on purity, viability and yield of selected CD34 cells. A target of >90% purity and >90% viable CD34+ cells is preferred. While lower Ficoll densities resulted in greater purity and fewer dead cells, it was surprisingly found that a greater portion of the CD34+ cells present in the deceased donor whole bone marrow before selection are lost using the lower Ficoll densities to prepare buffy coat. Thus, the high viability and purity of CD45/CD34+ cells achieved at the conventional Ficoll density gradient also leads to a large loss in yield (approximately 60% loss of input CD34+ cells).

Thus, in accordance with one aspect of the present disclosure, for freshly prepared the optimal density of Ficoll for selection of CD45/CD34+ cells at >90% purity and viability is less than 1.077 and particularly 1.063-1.052. This Ficoll density provides a higher yield of CD45/CD34+ cells with similar purity and cell viability to the conventional Ficoll density approach.

In another aspect of the present disclosure, the CD34+ cells can be initially acquired from a freshly prepared deceased donor bone marrow using the reduced density Ficoll-Paque described above. The BM can be cryogenically frozen and then the CD34+ cells can be acquired later using conventional density Ficoll-Paque. This approach essentially allows selective recovery of cells from deceased donor bone marrow—either before freezing using the modified Ficoll density or after freezing and thawing using conventional Ficoll density.

Once CD34+ have been isolated from bone marrow product, it could be said that the remaining cell product is enriched for MSCs, and especially vBM-MSCs.

Recovery of MSCs from Processed Bone

In another feature of the systems and methods disclosed herein, a method is provided for preparing a composition of cadaveric human MSCs from bone. In some embodiments, the preparation may include providing a bone derived from a deceased donor, grinding the bone into one or more ground bone segments, filtering the one or more ground bone segments and extracting the cadaveric human MSCs from the one or more ground bone segments. In some embodiments, the MSCs may be recovered from thawed or cryopreserved vertebral body bone fragments. In some embodiments, the extracted cadaveric human MSCs may be vertebral bone marrow MSCs (vBM-MSCs), adherent vertebral body mesenchymal stem cells (vBA-MSCs), or both. In some embodiments, the extracted cadaveric human MSCs are derived from a bone or fragments thereof that has already been processed to remove bone marrow or derivates thereof associated with the bone or fragment thereof (e.g. bone marrow derived cells, hematopoietic stem cells). In some embodiments, the extracted cadaveric human MSCs are derived from a bone or fragments thereof that has been processed for bone marrow and/or bone marrow-derived cells (e.g. hematopoietic stem cells) as described herein. In some embodiments, the extracted cadaveric human MSCs are derived from the bone grindings and/or segments described herein following filtration and/or extraction and/or isolation of bone marrow and/or bone marrow-derived cells as described herein. The processing and extraction of viable vBA-MSCs from the bone and/or derivates thereof (e.g. bone grindings described herein, bone segments described herein) results in significant improvements in cell yield, especially with respect to total cell yield (vBA-MSCs and hematopoietic stem cells) per weight of bone derived from a donor, and viability of cells with respect to the state of the art. In some embodiments, the vBA-MSCs described herein can be combined with bone marrow-derived MSCs (vBM-MSCs) isolated from bone marrow isolated and processed as described herein.

In some embodiments, the extraction of cadaveric human vBA-MSCs may include contacting the bone or derivatives thereof with a digestion solution. In some embodiments, the digestion solution may include one or more distinct enzymes. In some embodiments, the one or more distinct enzymes may include one or more collagenases and neutral proteases. In some embodiments, the digestion solution may be present at a ratio of volume to weight of the one or more ground bone segments and enzymatic solution of about 1:1 to about 15:1. In some embodiments, the ratio may be 1:1, 2.5:1, 5:1, 7.5:1, 10:1 and 15:1 (volume:weight). In some embodiments, the combination of one or more collagenases and neutral proteases is used to obtain the highest possible yields of vBA-MSC.

In some embodiments, a collagenase may include Clostridium histolyticum further comprising two active isoforms, C1 and C2. In some embodiments, one or more collagenases comprising isoforms C1 and C2 may be present in the digestion solution at a ratio comprising more collagenase isoform C1 than collagenase isoform C2. In some embodiments, the ratio of collagenase isoform C1 to collagenase isoform C2 may be about 30 to about 70: about 10 to about 29. In some embodiments, the ratio of collagenase isoform C1 to collagenase C2 may be 35:15. In some embodiments, the mass ratio of C1 and C2 for each concentration may be 70:30, 54:46, 37:63, 82:18, 54:46, and 90:10.

In some embodiments, the neutral protease may be Paneibacillus polymyxa neutral protease. In some embodiments, the neutral protease concentration may be about 2 U/ml to about 21 U/ml. In some embodiments, the neutral protease concentration may be about 2 U/ml to about 7 U/ml, about 2 U/ml to about 12 U/ml, about 2 U/ml to about 17 U/ml, about 2 U/ml to about 21 U/ml, about 7 U/ml to about 12 U/ml, about 7 U/ml to about 17 U/ml, about 7 U/ml to about 21 U/ml, about 12 U/ml to about 17 U/ml, about 12 U/ml to about 21 U/ml, or about 17 U/ml to about 21 U/ml. In some embodiments, the neutral protease concentration may be about 2 U/ml, about 7 U/ml, about 12 U/ml, about 17 U/ml, or about 21 U/ml. In some embodiments, the neutral protease concentration may be at least about 2 U/ml, about 7 U/ml, about 12 U/ml, or about 17 U/ml. In some embodiments, the neutral protease concentration may be at most about 7 U/ml, about 12 U/ml, about 17 U/ml, or about 21 U/ml. In some embodiments, the digestion solution may comprise the neutral protease at an activity of about 19.6 U/ml.

In some embodiments, the collagenase concentration is about 0.05 U/ml to about 1.6 U/ml. In some embodiments, the collagenase concentration is about 0.05 U/ml to about 0.1 U/ml, about 0.05 U/ml to about 0.15 U/ml, about 0.05 U/ml to about 0.2 U/ml, about 0.05 U/ml to about 0.25 U/ml, about 0.05 U/ml to about 0.3 U/ml, about 0.05 U/ml to about 0.35 U/ml, about 0.05 U/ml to about 0.4 U/ml, about 0.05 U/ml to about 0.8 U/ml, about 0.05 U/ml to about 1.2 U/ml, about 0.05 U/ml to about 1.6 U/ml, about 0.1 U/ml to about 0.15 U/ml, about 0.1 U/ml to about 0.2 U/ml, about 0.1 U/ml to about 0.25 U/ml, about 0.1 U/ml to about 0.3 U/ml, about 0.1 U/ml to about 0.35 U/ml, about 0.1 U/ml to about 0.4 U/ml, about 0.1 U/ml to about 0.8 U/ml, about 0.1 U/ml to about 1.2 U/ml, about 0.1 U/ml to about 1.6 U/ml, about 0.15 U/ml to about 0.2 U/ml, about 0.15 U/ml to about 0.25 U/ml, about 0.15 U/ml to about 0.3 U/ml, about 0.15 U/ml to about 0.35 U/ml, about 0.15 U/ml to about 0.4 U/ml, about 0.15 U/ml to about 0.8 U/ml, about 0.15 U/ml to about 1.2 U/ml, about 0.15 U/ml to about 1.6 U/ml, about 0.2 U/ml to about 0.25 U/ml, about 0.2 U/ml to about 0.3 U/ml, about 0.2 U/ml to about 0.35 U/ml, about 0.2 U/ml to about 0.4 U/ml, about 0.2 U/ml to about 0.8 U/ml, about 0.2 U/ml to about 1.2 U/ml, about 0.2 U/ml to about 1.6 U/ml, about 0.25 U/ml to about 0.3 U/ml, about 0.25 U/ml to about 0.35 U/ml, about 0.25 U/ml to about 0.4 U/ml, about 0.25 U/ml to about 0.8 U/ml, about 0.25 U/ml to about 1.2 U/ml, about 0.25 U/ml to about 1.6 U/ml, about 0.3 U/ml to about 0.35 U/ml, about 0.3 U/ml to about 0.4 U/ml, about 0.3 U/ml to about 0.8 U/ml, about 0.3 U/ml to about 1.2 U/ml, about 0.3 U/ml to about 1.6 U/ml, about 0.35 U/ml to about 0.4 U/ml, about 0.35 U/ml to about 0.8 U/ml, about 0.35 U/ml to about 1.2 U/ml, about 0.35 U/ml to about 1.6 U/ml, about 0.4 U/ml to about 0.8 U/ml, about 0.4 U/ml to about 1.2 U/ml, about 0.4 U/ml to about 1.6 U/ml, about 0.8 U/ml to about 1.2 U/ml, about 0.8 U/ml to about 1.6 U/ml, or about 1.2 U/ml to about 1.6 U/ml. In some embodiments, the collagenase concentration is about 0.05 U/ml, about 0.1 U/ml, about 0.15 U/ml, about 0.2 U/ml, about 0.25 U/ml, about 0.3 U/ml, about 0.35 U/ml, about 0.4 U/ml, about 0.8 U/ml, about 1.2 U/ml, or about 1.6 U/ml. In some embodiments, the collagenase concentration is at least about 0.05 U/ml, about 0.1 U/ml, about 0.15 U/ml, about 0.2 U/ml, about 0.25 U/ml, about 0.3 U/ml, about 0.35 U/ml, about 0.4 U/ml, about 0.8 U/ml, or about 1.2 U/ml. In some embodiments, the collagenase concentration is at most about 0.1 U/ml, about 0.15 U/ml, about 0.2 U/ml, about 0.25 U/ml, about 0.3 U/ml, about 0.35 U/ml, about 0.4 U/ml, about 0.8 U/ml, about 1.2 U/ml, or about 1.6 U/ml.

In accordance with one aspect of the disclosure, neutral protease concentration and collagenase concentrations (C1 and C2 collagenase) and ratio of solution volume (mls) to bone fragment weight (mgs) are determined.

In some embodiments, the total collagenase concentrations (C1 and C2 collagenase) are about 25 μg/ml to about 100 μg/ml. In some embodiments, the total collagenase concentrations are about 25 μg/ml to about 32.5 μg/ml, about 25 μg/ml to about 47.5 μg/ml, about 25 μg/ml to about 42.5 μg/ml, about 25 μg/ml to about 50 μg/ml, about 25 μg/ml to about 65 μg/ml, about 25 μg/ml to about 77.5 μg/ml, about 25 μg/ml to about 85 μg/ml, about 25 μg/ml to about 100 μg/ml, about 32.5 μg/ml to about 47.5 μg/ml, about 32.5 μg/ml to about 42.5 μg/ml, about 32.5 μg/ml to about 50 μg/ml, about 32.5 μg/ml to about 65 μg/ml, about 32.5 μg/ml to about 77.5 μg/ml, about 32.5 μg/ml to about 85 μg/ml, about 32.5 μg/ml to about 100 μg/ml, about 47.5 μg/ml to about 42.5 μg/ml, about 47.5 μg/ml to about 50 μg/ml, about 47.5 μg/ml to about 65 μg/ml, about 47.5 μg/ml to about 77.5 μg/ml, about 47.5 μg/ml to about 85 μg/ml, about 47.5 μg/ml to about 100 μg/ml, about 42.5 μg/ml to about 50 μg/ml, about 42.5 μg/ml to about 65 μg/ml, about 42.5 μg/ml to about 77.5 μg/ml, about 42.5 μg/ml to about 85 μg/ml, about 42.5 μg/ml to about 100 μg/ml, about 50 μg/ml to about 65 μg/ml, about 50 μg/ml to about 77.5 μg/ml, about 50 μg/ml to about 85 μg/ml, about 50 μg/ml to about 100 μg/ml, about 65 μg/ml to about 77.5 μg/ml, about 65 μg/ml to about 85 μg/ml, about 65 μg/ml to about 100 μg/ml, about 77.5 μg/ml to about 85 μg/ml, about 77.5 μg/ml to about 100 μg/ml, or about 85 μg/ml to about 100 μg/ml. In some embodiments, the total collagenase concentrations are about 25 μg/ml, about 32.5 μg/ml, about 47.5 μg/ml, about 42.5 μg/ml, about 50 μg/ml, about 65 μg/ml, about 77.5 μg/ml, about 85 μg/ml, or about 100 μg/ml. In some embodiments, the total collagenase concentrations are at least about 25 μg/ml, about 32.5 μg/ml, about 47.5 μg/ml, about 42.5 μg/ml, about 50 μg/ml, about 65 μg/ml, about 77.5 μg/ml, or about 85 μg/ml. In some embodiments, the total collagenase concentrations are at most about 32.5 μg/ml, about 47.5 μg/ml, about 42.5 μg/ml, about 50 μg/ml, about 65 μg/ml, about 77.5 μg/ml, about 85 μg/ml, or about 100 μg/ml.

In some embodiments, the mass ratio of C1 and C2 for each concentration are 70:30, 54:46, 37:63, 82:18 and 90:10, respectively.

According to the process, fragments of VB bone are placed in cryoprotectant solution comprised of PLASMA-LYTE™, 2.5% human serum albumin and 10% dimethyl sulfoxide (DMSO) and incubated for 1 hour at 4° C. In some embodiments, the incubation period is about 1 hour to about 3 hours. In some embodiments, the incubation period is about 1 hour to about 1.5 hours, about 1 hour to about 2 hours, about 1 hour to about 2.5 hours, about 1 hour to about 3 hours, about 1.5 hours to about 2 hours, about 1.5 hours to about 2.5 hours, about 1.5 hours to about 3 hours, about 2 hours to about 2.5 hours, about 2 hours to about 3 hours, or about 2.5 hours to about 3 hours. In some embodiments, the incubation period is about 1 hour, about 1.5 hours, about 2 hours, about 2.5 hours, or about 3 hours. In some embodiments, the incubation period is at least about 1 hour, about 1.5 hours, about 2 hours, or about 2.5 hours. In some embodiments, the incubation period is at most about 1.5 hours, about 2 hours, about 2.5 hours, or about 3 hours. The solution is removed and the bone fragments cooled at a rate of ˜1°/min to −86° C. and then plunged into liquid nitrogen. After 24-48 hours in liquid nitrogen, the bone fragments are thawed rapidly in a water bath set at 37° C. and then washed in saline and digested using the collagenase/protease solution described above.

In some embodiments, the volume-to-weight ratio was 5:1 at an incubation time of 2.5 hours. In some embodiments, the protease produced neutral protease activity of 19.6 U/ml.

The mixture of cells liberated by digesting VB bone fragment is cultured on tissue-coated plastic in the presence of Mesencult medium to select proliferative vBA-MSC. Freshly digested preparations as well as different passages of VB-MSC can be characterized by flow cytometry, colony forming unit-fibroblast (CFU-F) potential, population doubling time (PDT) and trilineage (adipogenic, chondrogenic, and osteogenic) differentiation in vitro.

In some embodiments, the method of cadaveric human MSC extraction disclosed herein may be capable of extracting quantities of about 10 million to about 10 billion. In some embodiments, cadaveric human MSCs may be administered in quantities of about 10 million to about 100 million, about 10 million to about 1 billion, about 10 million to about 10 billion, about 100 million to about 1 billion, about 100 million to about 10 billion, or about 1 billion to about 10 billion. In some embodiments, cadaveric human MSCs may be administered in quantities of about 10 million, about 100 million, about 1 billion, or about 10 billion. In some embodiments, cadaveric human MSCs may be administered in quantities of at least about 10 million, about 100 million, or about 1 billion. In some embodiments, cadaveric human MSCs may be administered in quantities of at most about 100 million, about 1 billion, or about 10 billion.

### Overview of Manufacturing Processes of Interferon γ-Primed Mesenchymal Stromal Cells

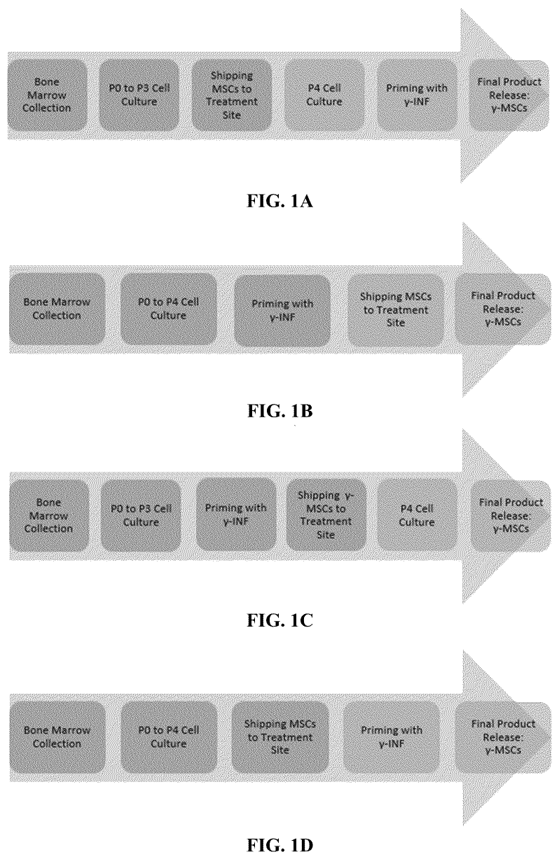

FIG. 1A to FIG. 1D are a schematics showing illustrative manufacturing processes for interferon γ-primed mesenchymal stromal cells (γMSCs). The schematics are to be understood according to the teachings presented in the present disclosure. As example, prior sections of this disclosure describe various processes involved in the “bone marrow collection” step. Any processes disclosed herein that relate to obtaining MSCs from bone marrow are include in the “bone marrow collection”. Similarly, as examples, P0, P1, P2, P3, and P4 cell culturing steps are disclosed below as well as method for priming MSCs with y-INF. Accordingly, FIG. 1A to FIG. 1D provide general methodologies that include the specific methods, steps, reagents, and the like that are disclosed in detail herein.

### Culturing of MSCs

The MSCs may be expanded in culture prior to being IFNγ primed. Culture-expanded MSCs have potent immunosuppressive activity mediated via a variety of cell to cell contact and soluble factors such as IDO, PGE2, TSG-6, CCL-2 and PD-L1, and TGFβ. These mediators inhibit T and B lymphocyte, NK cell and dendritic cell activation and function. MSCs also facilitate endogenous tissue repair and regeneration through secretion of cytokines such as VEGF, IL-6, IL-11, GM-CSF and SCF. As MSCs seem to be hypoimmunogenic and do not express co-stimulatory molecules, they can be infused into major histocompatibility complex (MHC) disparate recipients. Without wishing to be bound by theory, these beneficial properties suggest that MSCs may be useful for prevention and treatment of acute GVHD.

In one aspect of the present disclosure, extracted MSCs may be (e.g., vBA-MSCs and/or vBM-MSCs) cultured and passaged to realize clinical scale MSC preparation having a desired number of MSCs with the antigen profiles taught herein. In some embodiments, a clinical scale preparation may be obtained by serial passage expansion where each passage includes a step of splitting the previous culture into a plurality of cultures at a given ratio. Each passaging step increases the number of concurrent cultures in the preparation. In some embodiments, clinical scale preparations having the instant preparation profiles, e.g. antigen profile, TNFRI profile, cryopreservation profile, differentiation profile, and/or sterility (with respect to pathogens) are successfully produced.

In some embodiments, extracted MSCs are cultured in a medium wherein the medium is configured to generate MSCs having the instant preparation profiles, e.g. antigen profile, TNFRI profile, cryopreservation profile, differentiation profile, and/or sterility (with respect to pathogens). In some embodiments, the medium comprises minimal essential medium (MEM).

In some embodiments, the medium comprises alpha MEM. In some embodiments, the medium comprises human platelet lysate (hPL), e.g., Stemulate™. In some embodiments, the medium comprises fibroblast growth factor (FGF, e.g., carrier free FGF and/or FGF-2). In some embodiments, the medium comprises epidermal growth factor (EGF; e.g., carrier free EGF). In some embodiments, the medium comprises alpha MEM, hPL, FGF, EGF, or any combination thereof. In some embodiments, the medium comprises alpha MEM, hPL, FGF, and EGF. In some embodiments, the medium does not further require heparin.

In some embodiments, hPL is present in the medium at about 1% to about 21%. In some embodiments, hPL is present in the medium at about 1% to about 3%, about 1% to about 5%, about 1% to about 7%, about 1% to about 9%, about 1% to about 10%, about 1% to about 11%, about 1% to about 13%, about 1% to about 15%, about 1% to about 17%, about 1% to about 19%, about 1% to about 21%, about 3% to about 5%, about 3% to about 7%, about 3% to about 9%, about 3% to about 10%, about 3% to about 11%, about 3% to about 13%, about 3% to about 15%, about 3% to about 17%, about 3% to about 19%, about 3% to about 21%, about 5% to about 7%, about 5% to about 9%, about 5% to about 10%, about 5% to about 11%, about 5% to about 13%, about 5% to about 15%, about 5% to about 17%, about 5% to about 19%, about 5% to about 21%, about 7% to about 9%, about 7% to about 10%, about 7% to about 11%, about 7% to about 13%, about 7% to about 15%, about 7% to about 17%, about 7% to about 19%, about 7% to about 21%, about 9% to about 10%, about 9% to about 11%, about 9% to about 13%, about 9% to about 15%, about 9% to about 17%, about 9% to about 19%, about 9% to about 21%, about 10% to about 11%, about 10% to about 13%, about 10% to about 15%, about 10% to about 17%, about 10% to about 19%, about 10% to about 21%, about 11% to about 13%, about 11% to about 15%, about 11% to about 17%, about 11% to about 19%, about 11% to about 21%, about 13% to about 15%, about 13% to about 17%, about 13% to about 19%, about 13% to about 21%, about 15% to about 17%, about 15% to about 19%, about 15% to about 21%, about 17% to about 19%, about 17% to about 21%, or about 19% to about 21%. In some embodiments, hPL is present in the medium at about 1%, about 3%, about 5%, about 7%, about 9%, about 10%, about 11%, about 13%, about 15%, about 17%, about 19%, or about 21%. In some embodiments, hPL is present in the medium at least about 1%, about 3%, about 5%, about 7%, about 9%, about 10%, about 11%, about 13%, about 15%, about 17%, or about 19%. In some embodiments, hPL is present in the medium at most about 3%, about 5%, about 7%, about 9%, about 10%, about 11%, about 13%, about 15%, about 17%, about 19%, or about 21%.In some embodiments, FGF is present in the medium at about 0.5 ng/ml to about 5 ng/ml. In some embodiments, FGF is present in the medium at about 0.5 ng/ml to about 1 ng/ml, about 0.5 ng/ml to about 1.5 ng/ml, about 0.5 ng/ml to about 2 ng/ml, about 0.5 ng/ml to about 2.5 ng/ml, about 0.5 ng/ml to about 3 ng/ml, about 0.5 ng/ml to about 3.5 ng/ml, about 0.5 ng/ml to about 4 ng/ml, about 0.5 ng/ml to about 4.5 ng/ml, about 0.5 ng/ml to about 5 ng/ml, about 1 ng/ml to about 1.5 ng/ml, about 1 ng/ml to about 2 ng/ml, about 1 ng/ml to about 2.5 ng/ml, about 1 ng/ml to about 3 ng/ml, about 1 ng/ml to about 3.5 ng/ml, about 1 ng/ml to about 4 ng/ml, about 1 ng/ml to about 4.5 ng/ml, about 1 ng/ml to about 5 ng/ml, about 1.5 ng/ml to about 2 ng/ml, about 1.5 ng/ml to about 2.5 ng/ml, about 1.5 ng/ml to about 3 ng/ml, about 1.5 ng/ml to about 3.5 ng/ml, about 1.5 ng/ml to about 4 ng/ml, about 1.5 ng/ml to about 4.5 ng/ml, about 1.5 ng/ml to about 5 ng/ml, about 2 ng/ml to about 2.5 ng/ml, about 2 ng/ml to about 3 ng/ml, about 2 ng/ml to about 3.5 ng/ml, about 2 ng/ml to about 4 ng/ml, about 2 ng/ml to about 4.5 ng/ml, about 2 ng/ml to about 5 ng/ml, about 2.5 ng/ml to about 3 ng/ml, about 2.5 ng/ml to about 3.5 ng/ml, about 2.5 ng/ml to about 4 ng/ml, about 2.5 ng/ml to about 4.5 ng/ml, about 2.5 ng/ml to about 5 ng/ml, about 3 ng/ml to about 3.5 ng/ml, about 3 ng/ml to about 4 ng/ml, about 3 ng/ml to about 4.5 ng/ml, about 3 ng/ml to about 5 ng/ml, about 3.5 ng/ml to about 4 ng/ml, about 3.5 ng/ml to about 4.5 ng/ml, about 3.5 ng/ml to about 5 ng/ml, about 4 ng/ml to about 4.5 ng/ml, about 4 ng/ml to about 5 ng/ml, or about 4.5 ng/ml to about 5 ng/ml. In some embodiments, FGF is present in the medium at about 0.5 ng/ml, about 1 ng/ml, about 1.5 ng/ml, about 2 ng/ml, about 2.5 ng/ml, about 3 ng/ml, about 3.5 ng/ml, about 4 ng/ml, about 4.5 ng/ml, or about 5 ng/ml. In some embodiments, FGF is present in the medium at least about 0.5 ng/ml, about 1 ng/ml, about 1.5 ng/ml, about 2 ng/ml, about 2.5 ng/ml, about 3 ng/ml, about 3.5 ng/ml, about 4 ng/ml, or about 4.5 ng/ml. In some embodiments, FGF is present in the medium at most about 1 ng/ml, about 1.5 ng/ml, about 2 ng/ml, about 2.5 ng/ml, about 3 ng/ml, about 3.5 ng/ml, about 4 ng/ml, about 4.5 ng/ml, or about 5 ng/ml.

In some embodiments, EGF is present in the medium at about 0.5 ng/ml to about 5 ng/ml. In some embodiments, EGF is present in the medium at about 0.5 ng/ml to about 1 ng/ml, about 0.5 ng/ml to about 1.5 ng/ml, about 0.5 ng/ml to about 2 ng/ml, about 0.5 ng/ml to about 2.5 ng/ml, about 0.5 ng/ml to about 3 ng/ml, about 0.5 ng/ml to about 3.5 ng/ml, about 0.5 ng/ml to about 4 ng/ml, about 0.5 ng/ml to about 4.5 ng/ml, about 0.5 ng/ml to about 5 ng/ml, about 1 ng/ml to about 1.5 ng/ml, about 1 ng/ml to about 2 ng/ml, about 1 ng/ml to about 2.5 ng/ml, about 1 ng/ml to about 3 ng/ml, about 1 ng/ml to about 3.5 ng/ml, about 1 ng/ml to about 4 ng/ml, about 1 ng/ml to about 4.5 ng/ml, about 1 ng/ml to about 5 ng/ml, about 1.5 ng/ml to about 2 ng/ml, about 1.5 ng/ml to about 2.5 ng/ml, about 1.5 ng/ml to about 3 ng/ml, about 1.5 ng/ml to about 3.5 ng/ml, about 1.5 ng/ml to about 4 ng/ml, about 1.5 ng/ml to about 4.5 ng/ml, about 1.5 ng/ml to about 5 ng/ml, about 2 ng/ml to about 2.5 ng/ml, about 2 ng/ml to about 3 ng/ml, about 2 ng/ml to about 3.5 ng/ml, about 2 ng/ml to about 4 ng/ml, about 2 ng/ml to about 4.5 ng/ml, about 2 ng/ml to about 5 ng/ml, about 2.5 ng/ml to about 3 ng/ml, about 2.5 ng/ml to about 3.5 ng/ml, about 2.5 ng/ml to about 4 ng/ml, about 2.5 ng/ml to about 4.5 ng/ml, about 2.5 ng/ml to about 5 ng/ml, about 3 ng/ml to about 3.5 ng/ml, about 3 ng/ml to about 4 ng/ml, about 3 ng/ml to about 4.5 ng/ml, about 3 ng/ml to about 5 ng/ml, about 3.5 ng/ml to about 4 ng/ml, about 3.5 ng/ml to about 4.5 ng/ml, about 3.5 ng/ml to about 5 ng/ml, about 4 ng/ml to about 4.5 ng/ml, about 4 ng/ml to about 5 ng/ml, or about 4.5 ng/ml to about 5 ng/ml. In some embodiments, EGF is present in the medium at about 0.5 ng/ml, about 1 ng/ml, about 1.5 ng/ml, about 2 ng/ml, about 2.5 ng/ml, about 3 ng/ml, about 3.5 ng/ml, about 4 ng/ml, about 4.5 ng/ml, or about 5 ng/ml. In some embodiments, EGF is present in the medium at least about 0.5 ng/ml, about 1 ng/ml, about 1.5 ng/ml, about 2 ng/ml, about 2.5 ng/ml, about 3 ng/ml, about 3.5 ng/ml, about 4 ng/ml, or about 4.5 ng/ml. In some embodiments, EGF is present in the medium at most about 1 ng/ml, about 1.5 ng/ml, about 2 ng/ml, about 2.5 ng/ml, about 3 ng/ml, about 3.5 ng/ml, about 4 ng/ml, about 4.5 ng/ml, or about 5 ng/ml.

In some embodiments, the medium comprises a modified alpha MEM. In some embodiments, the modified alpha MEM comprises one or more inorganic salts, one or more amino acids, one or more vitamins, glucose, lipoic acid, sodium bicarbonate, sodium pyruvate, or any combination thereof.

In some embodiments, the one or more inorganic salts comprise calcium chloride (dihydrate), magnesium sulfate (heptahydrate), potassium chloride, sodium chloride, sodium phosphate monobasic (dehydrate), or any combination thereof. In some embodiments, each inorganic salt present in the medium is present at about 100 mg/Liter to about 800 mg/Liter. In some embodiments, each inorganic salt present in the medium is present at about 100 mg/Liter to about 200 mg/Liter, about 100 mg/Liter to about 300 mg/Liter, about 100 mg/Liter to about 400 mg/Liter, about 100 mg/Liter to about 500 mg/Liter, about 100 mg/Liter to about 600 mg/Liter, about 100 mg/Liter to about 700 mg/Liter, about 100 mg/Liter to about 800 mg/Liter, about 200 mg/Liter to about 300 mg/Liter, about 200 mg/Liter to about 400 mg/Liter, about 200 mg/Liter to about 500 mg/Liter, about 200 mg/Liter to about 600 mg/Liter, about 200 mg/Liter to about 700 mg/Liter, about 200 mg/Liter to about 800 mg/Liter, about 300 mg/Liter to about 400 mg/Liter, about 300 mg/Liter to about 500 mg/Liter, about 300 mg/Liter to about 600 mg/Liter, about 300 mg/Liter to about 700 mg/Liter, about 300 mg/Liter to about 800 mg/Liter, about 400 mg/Liter to about 500 mg/Liter, about 400 mg/Liter to about 600 mg/Liter, about 400 mg/Liter to about 700 mg/Liter, about 400 mg/Liter to about 800 mg/Liter, about 500 mg/Liter to about 600 mg/Liter, about 500 mg/Liter to about 700 mg/Liter, about 500 mg/Liter to about 800 mg/Liter, about 600 mg/Liter to about 700 mg/Liter, about 600 mg/Liter to about 800 mg/Liter, or about 700 mg/Liter to about 800 mg/Liter. In some embodiments, each inorganic salt present in the medium is present at about 100 mg/Liter, about 200 mg/Liter, about 300 mg/Liter, about 400 mg/Liter, about 500 mg/Liter, about 600 mg/Liter, about 700 mg/Liter, or about 800 mg/Liter. In some embodiments, each inorganic salt present in the medium is present at least about 100 mg/Liter, about 200 mg/Liter, about 300 mg/Liter, about 400 mg/Liter, about 500 mg/Liter, about 600 mg/Liter, or about 700 mg/Liter. In some embodiments, each inorganic salt present in the medium is present at most about 200 mg/Liter, about 300 mg/Liter, about 400 mg/Liter, about 500 mg/Liter, about 600 mg/Liter, about 700 mg/Liter, or about 800 mg/Liter.

In some embodiments, the one or more amino acids comprise glycine, alanine, alanyl-glutamine, arginine (HCl), asparagine (monohydrate), aspartic acid, cysteine (HCl) (monohydrate), cystine, glutamic acid, histidine, isoleucine, leucine, lysine, methionine, phenylalanine, proline, serine, threonine, tryptophan, tyrosine, valine, or any combination thereof. In some embodiments, the one or more amino acids are present in the L isoform. In some embodiments, the one or more amino acids are present in the D isoform. In some embodiments, the one or more amino acids are present in both isoforms. In some embodiments, each amino acid present in the medium is present at about 10 mg/Liter to about 100 mg/Liter. In some embodiments, each amino acid present in the medium is present at about 10 mg/Liter to about 20 mg/Liter, about 10 mg/Liter to about 30 mg/Liter, about 10 mg/Liter to about 40 mg/Liter, about 10 mg/Liter to about 50 mg/Liter, about 10 mg/Liter to about 60 mg/Liter, about 10 mg/Liter to about 70 mg/Liter, about 10 mg/Liter to about 80 mg/Liter, about 10 mg/Liter to about 90 mg/Liter, about 10 mg/Liter to about 100 mg/Liter, about 20 mg/Liter to about 30 mg/Liter, about 20 mg/Liter to about 40 mg/Liter, about 20 mg/Liter to about 50 mg/Liter, about 20 mg/Liter to about 60 mg/Liter, about 20 mg/Liter to about 70 mg/Liter, about 20 mg/Liter to about 80 mg/Liter, about 20 mg/Liter to about 90 mg/Liter, about 20 mg/Liter to about 100 mg/Liter, about 30 mg/Liter to about 40 mg/Liter, about 30 mg/Liter to about 50 mg/Liter, about 30 mg/Liter to about 60 mg/Liter, about 30 mg/Liter to about 70 mg/Liter, about 30 mg/Liter to about 80 mg/Liter, about 30 mg/Liter to about 90 mg/Liter, about 30 mg/Liter to about 100 mg/Liter, about 40 mg/Liter to about 50 mg/Liter, about 40 mg/Liter to about 60 mg/Liter, about 40 mg/Liter to about 70 mg/Liter, about 40 mg/Liter to about 80 mg/Liter, about 40 mg/Liter to about 90 mg/Liter, about 40 mg/Liter to about 100 mg/Liter, about 50 mg/Liter to about 60 mg/Liter, about 50 mg/Liter to about 70 mg/Liter, about 50 mg/Liter to about 80 mg/Liter, about 50 mg/Liter to about 90 mg/Liter, about 50 mg/Liter to about 100 mg/Liter, about 60 mg/Liter to about 70 mg/Liter, about 60 mg/Liter to about 80 mg/Liter, about 60 mg/Liter to about 90 mg/Liter, about 60 mg/Liter to about 100 mg/Liter, about 70 mg/Liter to about 80 mg/Liter, about 70 mg/Liter to about 90 mg/Liter, about 70 mg/Liter to about 100 mg/Liter, about 80 mg/Liter to about 90 mg/Liter, about 80 mg/Liter to about 100 mg/Liter, or about 90 mg/Liter to about 100 mg/Liter. In some embodiments, each amino acid present in the medium is present at about 10 mg/Liter, about 20 mg/Liter, about 30 mg/Liter, about 40 mg/Liter, about 50 mg/Liter, about 60 mg/Liter, about 70 mg/Liter, about 80 mg/Liter, about 90 mg/Liter, or about 100 mg/Liter. In some embodiments, each amino acid present in the medium is present at least about 10 mg/Liter, about 20 mg/Liter, about 30 mg/Liter, about 40 mg/Liter, about 50 mg/Liter, about 60 mg/Liter, about 70 mg/Liter, about 80 mg/Liter, or about 90 mg/Liter. In some embodiments, each amino acid present in the medium is present at most about 20 mg/Liter, about 30 mg/Liter, about 40 mg/Liter, about 50 mg/Liter, about 60 mg/Liter, about 70 mg/Liter, about 80 mg/Liter, about 90 mg/Liter, or about 100 mg/Liter. In some embodiments, each amino acid present in the medium is present at about 100 mg/Liter to about 500 mg/Liter. In some embodiments, each amino acid present in the medium is present at about 100 mg/Liter to about 200 mg/Liter, about 100 mg/Liter to about 300 mg/Liter, about 100 mg/Liter to about 400 mg/Liter, about 100 mg/Liter to about 500 mg/Liter, about 200 mg/Liter to about 300 mg/Liter, about 200 mg/Liter to about 400 mg/Liter, about 200 mg/Liter to about 500 mg/Liter, about 300 mg/Liter to about 400 mg/Liter, about 300 mg/Liter to about 500 mg/Liter, or about 400 mg/Liter to about 500 mg/Liter. In some embodiments, each amino acid present in the medium is present at about 100 mg/Liter, about 200 mg/Liter, about 300 mg/Liter, about 400 mg/Liter, or about 500 mg/Liter. In some embodiments, each amino acid present in the medium is present at least about 100 mg/Liter, about 200 mg/Liter, about 300 mg/Liter, or about 400 mg/Liter. In some embodiments, each amino acid present in the medium is present at most about 200 mg/Liter, about 300 mg/Liter, about 400 mg/Liter, or about 500 mg/Liter.

In some embodiments, the one or more vitamins comprise ascorbic acid, biotin, choline chloride, calcium pantothenate, folic acid, myo-inositol, niacinamide, pyridoxal (HCl), pyruvic acid (sodium salt), riboflavin, thiamine (HCl), vitamin B12, or any combination thereof. In some embodiments, the one or more vitamins are present in the L isoform. In some embodiments, the one or more vitamins are present in the D isoform. In some embodiments, the one or more vitamins are present in both isoforms. In some embodiments, each vitamin present in the medium is present at about 0.1 mg/Liter to about 2 mg/Liter. In some embodiments, each vitamin present in the medium is present at about 0.1 mg/Liter to about 0.3 mg/Liter, about 0.1 mg/Liter to about 0.5 mg/Liter, about 0.1 mg/Liter to about 0.7 mg/Liter, about 0.1 mg/Liter to about 0.9 mg/Liter, about 0.1 mg/Liter to about 1.1 mg/Liter, about 0.1 mg/Liter to about 1.3 mg/Liter, about 0.1 mg/Liter to about 1.5 mg/Liter, about 0.1 mg/Liter to about 1.7 mg/Liter, about 0.1 mg/Liter to about 1.9 mg/Liter, about 0.1 mg/Liter to about 2 mg/Liter, about 0.3 mg/Liter to about 0.5 mg/Liter, about 0.3 mg/Liter to about 0.7 mg/Liter, about 0.3 mg/Liter to about 0.9 mg/Liter, about 0.3 mg/Liter to about 1.1 mg/Liter, about 0.3 mg/Liter to about 1.3 mg/Liter, about 0.3 mg/Liter to about 1.5 mg/Liter, about 0.3 mg/Liter to about 1.7 mg/Liter, about 0.3 mg/Liter to about 1.9 mg/Liter, about 0.3 mg/Liter to about 2 mg/Liter, about 0.5 mg/Liter to about 0.7 mg/Liter, about 0.5 mg/Liter to about 0.9 mg/Liter, about 0.5 mg/Liter to about 1.1 mg/Liter, about 0.5 mg/Liter to about 1.3 mg/Liter, about 0.5 mg/Liter to about 1.5 mg/Liter, about 0.5 mg/Liter to about 1.7 mg/Liter, about 0.5 mg/Liter to about 1.9 mg/Liter, about 0.5 mg/Liter to about 2 mg/Liter, about 0.7 mg/Liter to about 0.9 mg/Liter, about 0.7 mg/Liter to about 1.1 mg/Liter, about 0.7 mg/Liter to about 1.3 mg/Liter, about 0.7 mg/Liter to about 1.5 mg/Liter, about 0.7 mg/Liter to about 1.7 mg/Liter, about 0.7 mg/Liter to about 1.9 mg/Liter, about 0.7 mg/Liter to about 2 mg/Liter, about 0.9 mg/Liter to about 1.1 mg/Liter, about 0.9 mg/Liter to about 1.3 mg/Liter, about 0.9 mg/Liter to about 1.5 mg/Liter, about 0.9 mg/Liter to about 1.7 mg/Liter, about 0.9 mg/Liter to about 1.9 mg/Liter, about 0.9 mg/Liter to about 2 mg/Liter, about 1.1 mg/Liter to about 1.3 mg/Liter, about 1.1 mg/Liter to about 1.5 mg/Liter, about 1.1 mg/Liter to about 1.7 mg/Liter, about 1.1 mg/Liter to about 1.9 mg/Liter, about 1.1 mg/Liter to about 2 mg/Liter, about 1.3 mg/Liter to about 1.5 mg/Liter, about 1.3 mg/Liter to about 1.7 mg/Liter, about 1.3 mg/Liter to about 1.9 mg/Liter, about 1.3 mg/Liter to about 2 mg/Liter, about 1.5 mg/Liter to about 1.7 mg/Liter, about 1.5 mg/Liter to about 1.9 mg/Liter, about 1.5 mg/Liter to about 2 mg/Liter, about 1.7 mg/Liter to about 1.9 mg/Liter, about 1.7 mg/Liter to about 2 mg/Liter, or about 1.9 mg/Liter to about 2 mg/Liter. In some embodiments, each vitamin present in the medium is present at about 0.1 mg/Liter, about 0.3 mg/Liter, about 0.5 mg/Liter, about 0.7 mg/Liter, about 0.9 mg/Liter, about 1.1 mg/Liter, about 1.3 mg/Liter, about 1.5 mg/Liter, about 1.7 mg/Liter, about 1.9 mg/Liter, or about 2 mg/Liter. In some embodiments, each vitamin present in the medium is present at least about 0.1 mg/Liter, about 0.3 mg/Liter, about 0.5 mg/Liter, about 0.7 mg/Liter, about 0.9 mg/Liter, about 1.1 mg/Liter, about 1.3 mg/Liter, about 1.5 mg/Liter, about 1.7 mg/Liter, or about 1.9 mg/Liter. In some embodiments, each vitamin present in the medium is present at most about 0.3 mg/Liter, about 0.5 mg/Liter, about 0.7 mg/Liter, about 0.9 mg/Liter, about 1.1 mg/Liter, about 1.3 mg/Liter, about 1.5 mg/Liter, about 1.7 mg/Liter, about 1.9 mg/Liter, or about 2 mg/Liter. In some embodiments, each vitamin present in the medium is present at about 10 mg/Liter to about 120 mg/Liter. In some embodiments, each vitamin present in the medium is present at about 10 mg/Liter to about 20 mg/Liter, about 10 mg/Liter to about 30 mg/Liter, about 10 mg/Liter to about 40 mg/Liter, about 10 mg/Liter to about 50 mg/Liter, about 10 mg/Liter to about 60 mg/Liter, about 10 mg/Liter to about 70 mg/Liter, about 10 mg/Liter to about 80 mg/Liter, about 10 mg/Liter to about 90 mg/Liter, about 10 mg/Liter to about 100 mg/Liter, about 10 mg/Liter to about 110 mg/Liter, about 10 mg/Liter to about 120 mg/Liter, about 20 mg/Liter to about 30 mg/Liter, about 20 mg/Liter to about 40 mg/Liter, about 20 mg/Liter to about 50 mg/Liter, about 20 mg/Liter to about 60 mg/Liter, about 20 mg/Liter to about 70 mg/Liter, about 20 mg/Liter to about 80 mg/Liter, about 20 mg/Liter to about 90 mg/Liter, about 20 mg/Liter to about 100 mg/Liter, about 20 mg/Liter to about 110 mg/Liter, about 20 mg/Liter to about 120 mg/Liter, about 30 mg/Liter to about 40 mg/Liter, about 30 mg/Liter to about 50 mg/Liter, about 30 mg/Liter to about 60 mg/Liter, about 30 mg/Liter to about 70 mg/Liter, about 30 mg/Liter to about 80 mg/Liter, about 30 mg/Liter to about 90 mg/Liter, about 30 mg/Liter to about 100 mg/Liter, about 30 mg/Liter to about 110 mg/Liter, about 30 mg/Liter to about 120 mg/Liter, about 40 mg/Liter to about 50 mg/Liter, about 40 mg/Liter to about 60 mg/Liter, about 40 mg/Liter to about 70 mg/Liter, about 40 mg/Liter to about 80 mg/Liter, about 40 mg/Liter to about 90 mg/Liter, about 40 mg/Liter to about 100 mg/Liter, about 40 mg/Liter to about 110 mg/Liter, about 40 mg/Liter to about 120 mg/Liter, about 50 mg/Liter to about 60 mg/Liter, about 50 mg/Liter to about 70 mg/Liter, about 50 mg/Liter to about 80 mg/Liter, about 50 mg/Liter to about 90 mg/Liter, about 50 mg/Liter to about 100 mg/Liter, about 50 mg/Liter to about 110 mg/Liter, about 50 mg/Liter to about 120 mg/Liter, about 60 mg/Liter to about 70 mg/Liter, about 60 mg/Liter to about 80 mg/Liter, about 60 mg/Liter to about 90 mg/Liter, about 60 mg/Liter to about 100 mg/Liter, about 60 mg/Liter to about 110 mg/Liter, about 60 mg/Liter to about 120 mg/Liter, about 70 mg/Liter to about 80 mg/Liter, about 70 mg/Liter to about 90 mg/Liter, about 70 mg/Liter to about 100 mg/Liter, about 70 mg/Liter to about 110 mg/Liter, about 70 mg/Liter to about 120 mg/Liter, about 80 mg/Liter to about 90 mg/Liter, about 80 mg/Liter to about 100 mg/Liter, about 80 mg/Liter to about 110 mg/Liter, about 80 mg/Liter to about 120 mg/Liter, about 90 mg/Liter to about 100 mg/Liter, about 90 mg/Liter to about 110 mg/Liter, about 90 mg/Liter to about 120 mg/Liter, about 100 mg/Liter to about 110 mg/Liter, about 100 mg/Liter to about 120 mg/Liter, or about 110 mg/Liter to about 120 mg/Liter. In some embodiments, each vitamin present in the medium is present at about 10 mg/Liter, about 20 mg/Liter, about 30 mg/Liter, about 40 mg/Liter, about 50 mg/Liter, about 60 mg/Liter, about 70 mg/Liter, about 80 mg/Liter, about 90 mg/Liter, about 100 mg/Liter, about 110 mg/Liter, or about 120 mg/Liter. In some embodiments, each vitamin present in the medium is present at least about 10 mg/Liter, about 20 mg/Liter, about 30 mg/Liter, about 40 mg/Liter, about 50 mg/Liter, about 60 mg/Liter, about 70 mg/Liter, about 80 mg/Liter, about 90 mg/Liter, about 100 mg/Liter, or about 110 mg/Liter.

In some embodiments, each vitamin present in the medium is present at most about 20 mg/Liter, about 30 mg/Liter, about 40 mg/Liter, about 50 mg/Liter, about 60 mg/Liter, about 70 mg/Liter, about 80 mg/Liter, about 90 mg/Liter, about 100 mg/Liter, about 110 mg/Liter, or about 120 mg/Liter.

In some embodiments, the glucose comprised in the medium is anhydrous. In some embodiments, the glucose is present in the L isoform. In some embodiments, the glucose is present in the D isoform. In some embodiments, the glucose is present in both isoforms. In some embodiments, glucose present in the medium is present at about 500 mg/Liter to about 1,600 mg/Liter. In some embodiments, glucose present in the medium is present at about 500 mg/Liter to about 600 mg/Liter, about 500 mg/Liter to about 700 mg/Liter, about 500 mg/Liter to about 800 mg/Liter, about 500 mg/Liter to about 900 mg/Liter, about 500 mg/Liter to about 1,000 mg/Liter, about 500 mg/Liter to about 1,100 mg/Liter, about 500 mg/Liter to about 1,200 mg/Liter, about 500 mg/Liter to about 1,300 mg/Liter, about 500 mg/Liter to about 1,400 mg/Liter, about 500 mg/Liter to about 1,500 mg/Liter, about 500 mg/Liter to about 1,600 mg/Liter, about 600 mg/Liter to about 700 mg/Liter, about 600 mg/Liter to about 800 mg/Liter, about 600 mg/Liter to about 900 mg/Liter, about 600 mg/Liter to about 1,000 mg/Liter, about 600 mg/Liter to about 1,100 mg/Liter, about 600 mg/Liter to about 1,200 mg/Liter, about 600 mg/Liter to about 1,300 mg/Liter, about 600 mg/Liter to about 1,400 mg/Liter, about 600 mg/Liter to about 1,500 mg/Liter, about 600 mg/Liter to about 1,600 mg/Liter, about 700 mg/Liter to about 800 mg/Liter, about 700 mg/Liter to about 900 mg/Liter, about 700 mg/Liter to about 1,000 mg/Liter, about 700 mg/Liter to about 1,100 mg/Liter, about 700 mg/Liter to about 1,200 mg/Liter, about 700 mg/Liter to about 1,300 mg/Liter, about 700 mg/Liter to about 1,400 mg/Liter, about 700 mg/Liter to about 1,500 mg/Liter, about 700 mg/Liter to about 1,600 mg/Liter, about 800 mg/Liter to about 900 mg/Liter, about 800 mg/Liter to about 1,000 mg/Liter, about 800 mg/Liter to about 1,100 mg/Liter, about 800 mg/Liter to about 1,200 mg/Liter, about 800 mg/Liter to about 1,300 mg/Liter, about 800 mg/Liter to about 1,400 mg/Liter, about 800 mg/Liter to about 1,500 mg/Liter, about 800 mg/Liter to about 1,600 mg/Liter, about 900 mg/Liter to about 1,000 mg/Liter, about 900 mg/Liter to about 1,100 mg/Liter, about 900 mg/Liter to about 1,200 mg/Liter, about 900 mg/Liter to about 1,300 mg/Liter, about 900 mg/Liter to about 1,400 mg/Liter, about 900 mg/Liter to about 1,500 mg/Liter, about 900 mg/Liter to about 1,600 mg/Liter, about 1,000 mg/Liter to about 1,100 mg/Liter, about 1,000 mg/Liter to about 1,200 mg/Liter, about 1,000 mg/Liter to about 1,300 mg/Liter, about 1,000 mg/Liter to about 1,400 mg/Liter, about 1,000 mg/Liter to about 1,500 mg/Liter, about 1,000 mg/Liter to about 1,600 mg/Liter, about 1,100 mg/Liter to about 1,200 mg/Liter, about 1,100 mg/Liter to about 1,300 mg/Liter, about 1,100 mg/Liter to about 1,400 mg/Liter, about 1,100 mg/Liter to about 1,500 mg/Liter, about 1,100 mg/Liter to about 1,600 mg/Liter, about 1,200 mg/Liter to about 1,300 mg/Liter, about 1,200 mg/Liter to about 1,400 mg/Liter, about 1,200 mg/Liter to about 1,500 mg/Liter, about 1,200 mg/Liter to about 1,600 mg/Liter, about 1,300 mg/Liter to about 1,400 mg/Liter, about 1,300 mg/Liter to about 1,500 mg/Liter, about 1,300 mg/Liter to about 1,600 mg/Liter, about 1,400 mg/Liter to about 1,500 mg/Liter, about 1,400 mg/Liter to about 1,600 mg/Liter, or about 1,500 mg/Liter to about 1,600 mg/Liter. In some embodiments, glucose present in the medium is present at about 500 mg/Liter, about 600 mg/Liter, about 700 mg/Liter, about 800 mg/Liter, about 900 mg/Liter, about 1,000 mg/Liter, about 1,100 mg/Liter, about 1,200 mg/Liter, about 1,300 mg/Liter, about 1,400 mg/Liter, about 1,500 mg/Liter, or about 1,600 mg/Liter. In some embodiments, glucose present in the medium is present at least about 500 mg/Liter, about 600 mg/Liter, about 700 mg/Liter, about 800 mg/Liter, about 900 mg/Liter, about 1,000 mg/Liter, about 1,100 mg/Liter, about 1,200 mg/Liter, about 1,300 mg/Liter, about 1,400 mg/Liter, or about 1,500 mg/Liter. In some embodiments, glucose present in the medium is present at most about 600 mg/Liter, about 700 mg/Liter, about 800 mg/Liter, about 900 mg/Liter, about 1,000 mg/Liter, about 1,100 mg/Liter, about 1,200 mg/Liter, about 1,300 mg/Liter, about 1,400 mg/Liter, about 1,500 mg/Liter, or about 1,600 mg/Liter.

In some embodiments, lipoic acid present in the medium is present at about 0.05 mg/Liter to about 0.5 mg/Liter. In some embodiments, the lipoic acid is present in the medium in the form of DL-thiotic acid. In some embodiments, lipoic acid present in the medium is present at about 0.05 mg/Liter to about 0.1 mg/Liter, about 0.05 mg/Liter to about 0.15 mg/Liter, about 0.05 mg/Liter to about 0.2 mg/Liter, about 0.05 mg/Liter to about 0.25 mg/Liter, about 0.05 mg/Liter to about 0.3 mg/Liter, about 0.05 mg/Liter to about 0.35 mg/Liter, about 0.05 mg/Liter to about 0.4 mg/Liter, about 0.05 mg/Liter to about 0.45 mg/Liter, about 0.05 mg/Liter to about 0.5 mg/Liter, about 0.1 mg/Liter to about 0.15 mg/Liter, about 0.1 mg/Liter to about 0.2 mg/Liter, about 0.1 mg/Liter to about 0.25 mg/Liter, about 0.1 mg/Liter to about 0.3 mg/Liter, about 0.1 mg/Liter to about 0.35 mg/Liter, about 0.1 mg/Liter to about 0.4 mg/Liter, about 0.1 mg/Liter to about 0.45 mg/Liter, about 0.1 mg/Liter to about 0.5 mg/Liter, about 0.15 mg/Liter to about 0.2 mg/Liter, about 0.15 mg/Liter to about 0.25 mg/Liter, about 0.15 mg/Liter to about 0.3 mg/Liter, about 0.15 mg/Liter to about 0.35 mg/Liter, about 0.15 mg/Liter to about 0.4 mg/Liter, about 0.15 mg/Liter to about 0.45 mg/Liter, about 0.15 mg/Liter to about 0.5 mg/Liter, about 0.2 mg/Liter to about 0.25 mg/Liter, about 0.2 mg/Liter to about 0.3 mg/Liter, about 0.2 mg/Liter to about 0.35 mg/Liter, about 0.2 mg/Liter to about 0.4 mg/Liter, about 0.2 mg/Liter to about 0.45 mg/Liter, about 0.2 mg/Liter to about 0.5 mg/Liter, about 0.25 mg/Liter to about 0.3 mg/Liter, about 0.25 mg/Liter to about 0.35 mg/Liter, about 0.25 mg/Liter to about 0.4 mg/Liter, about 0.25 mg/Liter to about 0.45 mg/Liter, about 0.25 mg/Liter to about 0.5 mg/Liter, about 0.3 mg/Liter to about 0.35 mg/Liter, about 0.3 mg/Liter to about 0.4 mg/Liter, about 0.3 mg/Liter to about 0.45 mg/Liter, about 0.3 mg/Liter to about 0.5 mg/Liter, about 0.35 mg/Liter to about 0.4 mg/Liter, about 0.35 mg/Liter to about 0.45 mg/Liter, about 0.35 mg/Liter to about 0.5 mg/Liter, about 0.4 mg/Liter to about 0.45 mg/Liter, about 0.4 mg/Liter to about 0.5 mg/Liter, or about 0.45 mg/Liter to about 0.5 mg/Liter. In some embodiments, lipoic acid present in the medium is present at about 0.05 mg/Liter, about 0.1 mg/Liter, about 0.15 mg/Liter, about 0.2 mg/Liter, about 0.25 mg/Liter, about 0.3 mg/Liter, about 0.35 mg/Liter, about 0.4 mg/Liter, about 0.45 mg/Liter, or about 0.5 mg/Liter. In some embodiments, lipoic acid present in the medium is present at least about 0.05 mg/Liter, about 0.1 mg/Liter, about 0.15 mg/Liter, about 0.2 mg/Liter, about 0.25 mg/Liter, about 0.3 mg/Liter, about 0.35 mg/Liter, about 0.4 mg/Liter, or about 0.45 mg/Liter. In some embodiments, lipoic acid present in the medium is present at most about 0.1 mg/Liter, about 0.15 mg/Liter, about 0.2 mg/Liter, about 0.25 mg/Liter, about 0.3 mg/Liter, about 0.35 mg/Liter, about 0.4 mg/Liter, about 0.45 mg/Liter, or about 0.5 mg/Liter.

In some embodiments, sodium bicarbonate present in the medium is present at about 250 mg/Liter to about 2,000 mg/Liter. In some embodiments, sodium bicarbonate present in the medium is present at about 250 mg/Liter to about 500 mg/Liter, about 250 mg/Liter to about 750 mg/Liter, about 250 mg/Liter to about 1,000 mg/Liter, about 250 mg/Liter to about 1,250 mg/Liter, about 250 mg/Liter to about 1,500 mg/Liter, about 250 mg/Liter to about 1,750 mg/Liter, about 250 mg/Liter to about 2,000 mg/Liter, about 500 mg/Liter to about 750 mg/Liter, about 500 mg/Liter to about 1,000 mg/Liter, about 500 mg/Liter to about 1,250 mg/Liter, about 500 mg/Liter to about 1,500 mg/Liter, about 500 mg/Liter to about 1,750 mg/Liter, about 500 mg/Liter to about 2,000 mg/Liter, about 750 mg/Liter to about 1,000 mg/Liter, about 750 mg/Liter to about 1,250 mg/Liter, about 750 mg/Liter to about 1,500 mg/Liter, about 750 mg/Liter to about 1,750 mg/Liter, about 750 mg/Liter to about 2,000 mg/Liter, about 1,000 mg/Liter to about 1,250 mg/Liter, about 1,000 mg/Liter to about 1,500 mg/Liter, about 1,000 mg/Liter to about 1,750 mg/Liter, about 1,000 mg/Liter to about 2,000 mg/Liter, about 1,250 mg/Liter to about 1,500 mg/Liter, about 1,250 mg/Liter to about 1,750 mg/Liter, about 1,250 mg/Liter to about 2,000 mg/Liter, about 1,500 mg/Liter to about 1,750 mg/Liter, about 1,500 mg/Liter to about 2,000 mg/Liter, or about 1,750 mg/Liter to about 2,000 mg/Liter. In some embodiments, sodium bicarbonate present in the medium is present at about 250 mg/Liter, about 500 mg/Liter, about 750 mg/Liter, about 1,000 mg/Liter, about 1,250 mg/Liter, about 1,500 mg/Liter, about 1,750 mg/Liter, or about 2,000 mg/Liter. In some embodiments, sodium bicarbonate present in the medium is present at least about 250 mg/Liter, about 500 mg/Liter, about 750 mg/Liter, about 1,000 mg/Liter, about 1,250 mg/Liter, about 1,500 mg/Liter, or about 1,750 mg/Liter. In some embodiments, sodium bicarbonate present in the medium is present at most about 500 mg/Liter, about 750 mg/Liter, about 1,000 mg/Liter, about 1,250 mg/Liter, about 1,500 mg/Liter, about 1,750 mg/Liter, or about 2,000 mg/Liter.

In some embodiments, sodium pyruvate present in the medium is present at about 50 mg/Liter to about 160 mg/Liter. In some embodiments, sodium pyruvate present in the medium is present at about 50 mg/Liter to about 60 mg/Liter, about 50 mg/Liter to about 70 mg/Liter, about 50 mg/Liter to about 80 mg/Liter, about 50 mg/Liter to about 90 mg/Liter, about 50 mg/Liter to about 100 mg/Liter, about 50 mg/Liter to about 110 mg/Liter, about 50 mg/Liter to about 120 mg/Liter, about 50 mg/Liter to about 130 mg/Liter, about 50 mg/Liter to about 140 mg/Liter, about 50 mg/Liter to about 150 mg/Liter, about 50 mg/Liter to about 160 mg/Liter, about 60 mg/Liter to about 70 mg/Liter, about 60 mg/Liter to about 80 mg/Liter, about 60 mg/Liter to about 90 mg/Liter, about 60 mg/Liter to about 100 mg/Liter, about 60 mg/Liter to about 110 mg/Liter, about 60 mg/Liter to about 120 mg/Liter, about 60 mg/Liter to about 130 mg/Liter, about 60 mg/Liter to about 140 mg/Liter, about 60 mg/Liter to about 150 mg/Liter, about 60 mg/Liter to about 160 mg/Liter, about 70 mg/Liter to about 80 mg/Liter, about 70 mg/Liter to about 90 mg/Liter, about 70 mg/Liter to about 100 mg/Liter, about 70 mg/Liter to about 110 mg/Liter, about 70 mg/Liter to about 120 mg/Liter, about 70 mg/Liter to about 130 mg/Liter, about 70 mg/Liter to about 140 mg/Liter, about 70 mg/Liter to about 150 mg/Liter, about 70 mg/Liter to about 160 mg/Liter, about 80 mg/Liter to about 90 mg/Liter, about 80 mg/Liter to about 100 mg/Liter, about 80 mg/Liter to about 110 mg/Liter, about 80 mg/Liter to about 120 mg/Liter, about 80 mg/Liter to about 130 mg/Liter, about 80 mg/Liter to about 140 mg/Liter, about 80 mg/Liter to about 150 mg/Liter, about 80 mg/Liter to about 160 mg/Liter, about 90 mg/Liter to about 100 mg/Liter, about 90 mg/Liter to about 110 mg/Liter, about 90 mg/Liter to about 120 mg/Liter, about 90 mg/Liter to about 130 mg/Liter, about 90 mg/Liter to about 140 mg/Liter, about 90 mg/Liter to about 150 mg/Liter, about 90 mg/Liter to about 160 mg/Liter, about 100 mg/Liter to about 110 mg/Liter, about 100 mg/Liter to about 120 mg/Liter, about 100 mg/Liter to about 130 mg/Liter, about 100 mg/Liter to about 140 mg/Liter, about 100 mg/Liter to about 150 mg/Liter, about 100 mg/Liter to about 160 mg/Liter, about 110 mg/Liter to about 120 mg/Liter, about 110 mg/Liter to about 130 mg/Liter, about 110 mg/Liter to about 140 mg/Liter, about 110 mg/Liter to about 150 mg/Liter, about 110 mg/Liter to about 160 mg/Liter, about 120 mg/Liter to about 130 mg/Liter, about 120 mg/Liter to about 140 mg/Liter, about 120 mg/Liter to about 150 mg/Liter, about 120 mg/Liter to about 160 mg/Liter, about 130 mg/Liter to about 140 mg/Liter, about 130 mg/Liter to about 150 mg/Liter, about 130 mg/Liter to about 160 mg/Liter, about 140 mg/Liter to about 150 mg/Liter, about 140 mg/Liter to about 160 mg/Liter, or about 150 mg/Liter to about 160 mg/Liter. In some embodiments, sodium pyruvate present in the medium is present at about 50 mg/Liter, about 60 mg/Liter, about 70 mg/Liter, about 80 mg/Liter, about 90 mg/Liter, about 100 mg/Liter, about 110 mg/Liter, about 120 mg/Liter, about 130 mg/Liter, about 140 mg/Liter, about 150 mg/Liter, or about 160 mg/Liter. In some embodiments, sodium pyruvate present in the medium is present at least about 50 mg/Liter, about 60 mg/Liter, about 70 mg/Liter, about 80 mg/Liter, about 90 mg/Liter, about 100 mg/Liter, about 110 mg/Liter, about 120 mg/Liter, about 130 mg/Liter, about 140 mg/Liter, or about 150 mg/Liter. In some embodiments, sodium pyruvate present in the medium is present at most about 60 mg/Liter, about 70 mg/Liter, about 80 mg/Liter, about 90 mg/Liter, about 100 mg/Liter, about 110 mg/Liter, about 120 mg/Liter, about 130 mg/Liter, about 140 mg/Liter, about 150 mg/Liter, or about 160 mg/Liter.

In some embodiments, the pH of the alpha MEM is between 7.0 and 7.4.

In some embodiments, the alpha MEM comprises the ingredients are presented in Table 2.

TABLE 2

| Components | mg/Liter |
|---|---|
| INORGANIC SALTS | |
| Calcium Chloride, dihydrate | 264 |
| (CaCl₂•2H₂O) | |
| Magnesium Sulfate, heptahydrate | 200 |
| (MgSO₄•7H₂0) | |
| Potassium Chloride (KCl) | 400 |
| Sodium Chloride (NaCl) | 6800 |
| Sodium Phosphate Monobasic, dihydrate | 158 |
| (NaF1₂1, 0₄•2H₂0) | |
| OTHER COMPONENTS | |
| D-Glucose, Anhydrous (C₆11₁₂0₆) | 1000 |
| Lipoic Acid (DL-Thiotic Acid) | 0.2 |
| (C81⁻1140252) | |
| Sodium Bicarbonate (NaHCO₃) | 2200 |
| Sodium Pyruvate (C₃H₃Na0₃) | 110 |
| AMINO ACIDS | |
| Glycine (C2H5NO2) | 500 |
| L-Alanine (C3H7NO2) | 250 |
| L-Alanyl-Glutamine (C₈H₁₅N₃0₄) | 406 |
| L-Arginine HCI (C₆H₁₅CIN₄0₂) | 105 |
| L-Asparagine, monohydrate | 500 |
| (C₄H₈N₂0₃•1 − 1₂0) | |
| L-Aspartic Acid (C4H7N04) | 300 |
| L-Cysteine HCI, monohydrate | 100 |
| (C₃H₇NO₂•HCI•H₂0) | |
| L-Cystine (C6Hi2N20452) | 240 |
| L-Glutamic Acid (C₅H₉N0₄) | 750 |
| L-Histidine (C6H9N302) | 310 |
| L-Isoleucine (C₆F1₁₃NO₂) | 52.4 |
| L-Leucine (C₆F1₁₃NO₂) | 52.4 |
| L-Lysine (C₆H₁₄N₂0₂) | 580 |
| L-Methionine (C₅H₁₁NO₂S) | 150 |
| L-Phenylalanine (C₉H₁₁NO₂) | 320 |
| L-Proline (C5H9NO2) | 400 |
| L-Serine (C₃H₇NO₃) | 250 |
| L-Threonine (C₄H₉NO₃) | 480 |
| L-Tryptophan | 100 |
| (C11H12N2O2) | |
| L-Tyrosine (C₉H₉NO₃) | 360 |
| L-Valine (C₅H₁₁NO₂) | 460 |
| VITAMINS | |
| L-Ascorbic Acid (C6H806) | 500 |
| D-Biotin (CᵢₒHᵢ₆N₂0₃S) | 0.1 |
| Choline Chloride | 10 |
| (C₅H₁₄CIN0) | |
| D-Calcium Pantothenate | 10 |
| (Ci8H32CaN201o) | |
| Folic Acid (C191⁻119N706) | 10 |
| Myo-Inositol (C₆11₁₂0₆) | 20 |
| Niacinamide (C₆H₆N₂0) | 10 |
| Pyridoxal HCI | 10 |
| (C₈H₉NO₃•FICI) | |
| Pyruvic Acid, Sodium Salt | 110 |
| (C₃H₃Na0₃) | |
| Riboflavin (C₁₇H_{2O}N₄0₆) | 0.1 |
| Thiamine HCI | 10 |
| (C₁₂H₁₇CIN₄0S•HCI) | |
| Vitamin B12 | 1.3 |
| (C63H88CON14°14P) | |

In some embodiments, the vBM-MSCs and/or vBA-MSCs (collectively MSCs) are cultured in an MSC Culture medium comprising alpha MEM as described in Table 2, 10% hPL (e.g., Stemulate™, 2 ng/ml recombinant, FGF (e.g., basic fibroblast growth factor (FGF-2) and/or carrier free FGF), and 2 ng/ml recombinant, epidermal growth factor (EGF, e.g., carrier free EGF). In some embodiments, no heparin is included in the culturing medium. In various embodiments, the MSCs are cultured for about 14 days with fresh media changes occurring every 3 to 4 days. Approximately 3.9 billion mononuclear cells (e.g., 2 to 5 billion, 3 to 4 billion, and any number of cells therebetween) are plated in culture dishes, e.g., six CellBIND® 10-chamber CellSTACKS®. In some embodiments, no bovine or porcine components and no antibiotics/mycotics are used in culturing process. The MSCs at this stage are termed Passage 0 (P0) cells.

When the P0 MSCs have achieved about 75% confluence or greater, the cells are detached and re-plated in MSC Culture Media. The MSCs at this stage are termed Passage 1 (P1) cells. Detachment, at this passage and/or any future passage, may comprise either or both of mechanical and enzymatic methods. The enzymatic method may comprise use of a trypsin or a commercially-available trypsin replacement, e.g., TrypLE™. In some embodiments, the P1 cells (about 410 million cells, e.g., 250 million to 500 million, 300 million to 450 million, or any number of cells therebetween) are cultured for 4 to 5 days and when the culture has reached about 75% confluence or greater, the cells are detached and resuspended in Plasma Lyte-A+2.5% human serum albumin (HSA)+5% dimethyl sulfoxide (DMSO) at about 13 million cells/ml and packaged in about 220 2-mL CellSeal® closed-system cryovials at 2 mL per cryovial using an automatic filler. The cryovials are then cryopreserved and placed into the vapor phase above liquid nitrogen in cryogenic tanks for storage at less than or equal to −140° C.

In alternate embodiments, the P1 cells are not cryopreserved; instead, are detached and re-plated in MSC Culture media to become the Passage 2 (P2) cells.

Table 3 lists testing optional performed on the P1 cells and preferable features that cultured MSCs may possess. In some cases, only subset of the tests listed in Table 3 are performed. As an example, Quantitative Transmission Electron Microscopy may be omitted.

TABLE 3
P1 MSC Characterization

| Attributes | Test | Preferable Features |
|---|---|---|
| Viability | Trypan Blue or Acridine | ≥70% |
| | Orange/Propidium Iodide | |
| Cell Product | Direct Inoculation Method | No Growth |
| Sterility | (determined suitable through | |
| | Bacteriostasis and Fungistasis | |
| | testing on test article) | |
| Mycoplasma | Rapid Detection of Mycoplasma by | Negative for Mycoplasma |
| | Real-Time PCR | |
| Adventitious | 28-Day In Vitro Assay (utilizing 3 | Negative for the Presence of |
| Agents | cell lines) | Viral Contaminants |
| | CoA Detection of 14 viruses by RT- | Negative |
| | PCR (Human Panel 1) | |
| | Quantitative Transmission Electron | No extraneous agents observed |
| | Microscopy of Sections for the | in the 200 cell profiles |
| | Detection of Viruses, Fungi, Yeasts, | examined |
| | Bacteria, and Mycoplasmas (200 | |
| | cell profiles) | |
| Cell Product | Flow Cytometry | CD90, CD105 positive |
| Identity and | | (≥90%), CD45, CD14 negative |
| Cellular Purity | | (≤5%) |
| Cell Product | Colony forming unit-fibroblast | Greater than 20,000 colonies |
| Activity | (CFU-F) analysis | per million cells plated in |
| | | preferred. |

Potency of P1 cells can be evaluated through colony forming unit-fibroblast (CFU-F) analysis. For this assay, cells are resuspended in medium and counted using a Cellometer or via Trypan Blue Manual Counting. Next, 50 cells each are pipetted into 2 wells of a 6 well tissue culture treated plate. The plate is incubated for 10-14 days or until colonies are above 75% confluence (e.g., 80% confluence), refeeding with prewarmed medium every 3-4 days. Once the desired confluence is met, medium is aspirated, the plate is washed twice with phosphate buffered saline (PBS), and 5 mL of Methanol is added to each well. This is allowed to sit for 5 minutes to fix the cells, then it is removed, and the wells are stained with Crystal Violet for 20 minutes. Finally, the Crystal Violet is removed, and the plates are washed 3 times with DI water, and colonies are counted (a colony must have 50 or more cells). An average of the two wells is taken and the average is used to calculate CFU-F colonies per 1M cells plated.

For P1 cells (or later stages), various methodologies can be used to detect mycoplasma including PCR, ELISA and biochemical reaction. All methods have proven reliable; however, PCR and ELISA methods are more labor intensive than biochemical methods. In some embodiments, the MycoAlert® Assay system (Lonza) is used to detect mycoplasma. The MycoAlert® Assay is a selective biochemical test that exploits the activity of certain mycoplasmal enzymes. The presence of these enzymes provides a rapid screening procedure, allowing sensitive detection of contaminating mycoplasma in a test sample. The viable mycoplasma is lysed and the enzymes react with the MycoAlert® Substrate catalyzing the conversion of ADP to ATP. By measuring the level of ATP in a sample both before and after the addition of the MycoAlert® Substrate, a ratio can be obtained which is indicative of the presence or absence of mycoplasma. If these enzymes are not present, the second reading shows no increase over the first, while reaction of mycoplasmal enzymes with their specific substrates in the MycoAlert® substrate leads to elevated ATP levels. Changes in ATP are detected using a bioluminescent reaction.

P1 cells, either thawed from frozen cryovials or freshly detached from the P1 culture vessel, are re-plated in MSC Culture media to become the Passage 2 (P2) cells. When the P2 cells have achieved about 75% confluence or greater, e.g., around four to five days, the cells are detached and re-plated in MSC Culture Media. In some embodiments, cells (about 410 million cells, e.g., 250 million to 500 million, 300 million to 450 million, or any number of cells therebetween) are re-plated in about 16 CellBind® 10-chamber CellStacks®. These cells are now termed Passage 3 or P3 cells.

In some embodiments, the P3 cells are cultured for 4-5 days and once the culture vessels have achieved about 75% confluence or greater, the cells are detached, resuspended in PlasmaLyte-A+2.5% HSA+5% DMSO at about 20 million cells/ml and packaged in 5-mL CellSeal® closed-system cryovials at 5 mL per cryovial using an automatic filler. The cryovials are then cryopreserved, which may comprise a precooling step or equilibration at 4° C. followed by passive cryopreservation at a rate of −1° C. per minute to about −80° C. or less; then the cryovials are placed into the vapor phase above liquid nitrogen in cryogenic tanks for storage at less than or equal to −140° C. Cryopreserved P3 cells may be shipped to a treatment site for further culturing and final product formulation to γ-MSC. See, e.g., FIG. 1A. In some cases, the MSC cells are collected from donor bone and initially cultured (P0 through P3) at the same location as the treatment site; in these cases, cryopreserved P3 cells do not need to be shipped to another site. These embodiments are variants of FIG. 1B and FIG. 1D in which the “Shipping” may be within a facility, campus, or healthcare complex.

In alternate embodiments, the P3 cells are not cryopreserved; instead, are detached and re-plated in MSC Culture media to become the Passage 4 (P4) cells. See, e.g., FIG. 1B and FIG. 1D.

Table 4 lists optional tests performed on the P3 cells and preferable features that cultured MSCs may possess. In some cases, only subset of the tests listed in Table 4 are performed.

TABLE 4
P3 MSC Characterization

| Attributes | Test | Preferable Features |
|---|---|---|
| Viability | Trypan Blue or Acridine | ≥70% |
| | Orange/Propidium Iodide | |
| Cell Product | Direct Inoculation Method | No Growth |
| Sterility | (determined suitable through | |
| | Bacteriostasis/Fungistasis testing | |
| | on test article), e.g., USP/CFR | |
| | 610.12 GMP | |
| Expended Media | Filtration method (determined | No Growth |
| Sterility | suitable through Bacteriostasis/ | |
| | Fungistasis testing on test article) | |
| Mycoplasma | Rapid Detection of Mycoplasma by | Negative for Mycoplasma |
| | Real-Time PCR | |
| Cell Product | Flow Cytometry | CD90, 105 positive (≥90%), |
| Identity and Purity | | CD45, CD14 negative (≤5%) |
| Cell Product | Colony forming unit-fibroblast | >20k colonies per 1M cells |
| Activity | (CFU-F) | plated |

Potency of P3 cells can be evaluated through colony forming unit-fibroblast (CFU-F) analysis. For this assay, cells are resuspended in medium and counted using a Cellometer or via Trypan Blue Manual Counting. Next, 50 cells each are pipetted into 2 wells of a 6 well tissue culture treated plate. The plate is incubated for 10-14 days or until colonies are at 80% confluence, refeeding with prewarmed medium every 3-4 days. Once confluent, medium is aspirated, the plate is washed twice with phosphate buffered saline (PBS), and 5 mL of Methanol is added to each well. This is allowed to sit for 5 minutes to fix the cells, then it is removed, and the wells are stained with Crystal Violet for 20 minutes. Finally, the Crystal Violet is removed, and the plates are washed 3 times with DI water, and colonies are counted (a colony must have 50 or more cells). An average of the two wells is taken and the average is used to calculate CFU-F colonies per 1M cells plated.

The time elapsed through each culture step is variable, with >75% confluence generally being achieved in about four to about five days. Time elapsed from a culture harvest to the start of the cryopreservation may be less than about six hours. Upon cryopreservation, cells are expected to be indefinitely stable.

In some embodiments, the primary MSCs may be further passaged to non-primary cells (e.g. removed from the culture surface and expanded into additional area) by seeding at a density of about 1,000 to about one million nucleated cells/cm² of culture dish (e.g. about 5,900 cells/cm² plus and minus about 1,200), and then culturing for additional days, e.g. about 14±about 2 days. In suitable embodiments, the primary cells may be grown to confluence, and in some instances may be passaged to a second culture of non-primary cells by seeding the primary cells from a confluent primary cell culture in the second culture surface in an amount below confluence and growing the non-primary culture to confluence. This method can be repeated for additional passages.

In some embodiments, the MSCs in the treatment composition may originate from sequential generation number (i.e., they are within about 1 or about 2 or about 3 or about 4 cell doublings of each other). Optionally, the average number of cell doublings in the present composition treatment composition may be about 20 to about 25 doublings. Optionally, the average number of cell doublings in the present treatment composition may be about 9 to about 13 (e.g., about 11 or about 11.2) doublings arising from the primary culture, plus about 1, about 2, about 3, or about 4 doublings per passage (for example, about 2.5 doublings per passage). Exemplary average cell doublings in present preparations may be of about 13.5, about 16, about 18.5, about 21, about 23.5, about 26, about 28.5, about 31, about 33.5, or about 36 when produced by about 1, about 2, about 3, about 4, about 5, about 6, about 7, about 8, about 9, or about 10 passages, respectively.

In some embodiments, notwithstanding one or more population doublings, the MSCs in the treatment composition (e.g. vBM-MSCs and/or vBA-MSCs; collectively MSCs) may originate from MSCs that were cultured through about 1, about 2, about 3, about 4, about 5, about 6, about 7, about 8, about 9, or about 10 passages.

Confluence refers to the percentage of the surface of a culture dish that is covered by adherent cells. For example, 50 percent confluence means roughly half of the surface is covered, while 100 percent confluence means the surface is completely covered by the cells, and no more room is left for the cells to grow as a monolayer. By “75% confluence or greater” is meant about 75% confluence, about 76% confluence, about 77% confluence, about 78% confluence, about 79% confluence, about 80% confluence, about 81% confluence, about 82% confluence, about 83% confluence, about 84% confluence, about 85% confluence, about 86% confluence, about 87% confluence, about 88% confluence, about 89% confluence, about 90% confluence, about 91% confluence, about 92% confluence, about 93% confluence, about 94% confluence, about 95% confluence, about 96% confluence, about 97% confluence, about 98% confluence, about 99% confluence, or about 100% confluence. Confluence can be determined by any standard method used in the field. See, e.g., Haenel, Frauke, and Norbert Garbow. “Cell counting and confluency analysis as quality controls in cell-based assays.” Multimode Detection (2014): 1-5.

### Cryopreservation and Rinse Media

The MSCs may undergo cryopreservation after completing a primary expansion. In some embodiments, for a method provided herein, said cell culture was cryopreserved in a cryopreservation media, wherein said cryopreservation media comprises an electrolyte formulation, human serum albumin (HSA), dimethyl sulfoxide (DMSO), or any combination thereof.

In some embodiments, cryovials are cryopreserved in a method comprising a precooling step or equilibration at 4° C. followed by passive cryopreservation at a rate of −1° C. per minute to about −80° C. or less; then the cryovials are placed into the vapor phase above liquid nitrogen in cryogenic tanks for storage at less than or equal to −140° C.

In some embodiments, said cryopreservation media comprises about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, or about 9% to about 10% HSA. In some embodiments, said cryopreservation media comprises about 1% to about 5% HSA. In some embodiments, said cryopreservation media comprises about 2.5% HSA.

In some embodiments, said cryopreservation media comprises about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, or about 9% to about 10% DMSO. In some embodiments, said cryopreservation media comprises about 1% to about 10% DMSO. In some embodiments, said cryopreservation media comprises about 5% DMSO.

In some embodiments, said electrolyte formulation is Plasmalyte A.

In some embodiments, for a method provided herein, the method further comprises, resuspending said cell culture in a rinse media, wherein said rinse media comprises an electrolyte formulation, human serum albumin (HSA), or both. In some embodiments, for a method provided herein, the method further comprises resuspending said cell culture in a rinse media, wherein said rinse media comprises an electrolyte formulation, human serum albumin (HSA), or both.

In some embodiments, the rinse media is fresh.

In some embodiments, said rinse media comprises about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, or about 9% to about 10% HSA. In some embodiments, said rinse media comprises about 1% to about 5% HSA. In some embodiments, said rinse media comprises about 2.5% HSA.

In some embodiments, said electrolyte formulation is Plasmalyte A.

In some embodiments, the systems and methods described herein have the ability to generate about 220 cryovials (26 million cells/vial) of passage 1 (P1) master cell bank (MCB), which can generate approximately 193,600 vials (100 million cells/vial) of passage 4 (P4) marrow-derived mesenchymal stem cell final product with the potential to treat about 193,600 subjects at a dose of 100 million cells/dose. In some embodiments, the systems and methods described herein generate between about 100, 120, 140, 160, 180, 200, 220, 240, 260, 280, 300, or more than 300 cryovials of passage 1 master cell bank. In some embodiments, each cryovial comprises about 10 million, about 11 million, about 12 million, about 13 million, about 14 million, about 15 million, about 16 million, about 17 million, about 18 million, about 19 million, about 20 million, about 21 million, about 22 million, about 23 million, about 24 million, about 25 million, about 26 million, about 27 million, about 28 million, about 29 million, about 30 million, about 32 million, about 32 million, about 33 million, about 34 million, about 35 million, about 36 million, about 37 million, about 38 million, about 48 million, or 40 million cells per vial of passage 1 master bank cells. In some embodiments, this allows the generation of about 100,000, about 110,000, about 120,000, about 130,000, about 140,000, about 150,000, about 160,000, about 170,000, about 180,000, about 190,000, about 200,000, about 210,000, about 220,000, about 230,000, about 240,000, about 250,000, about 260,000, about 270,000, about 280,000, about 290,000, about or 300,000 vials of passage 4 (P4) cells. In some embodiments, this allows the generation of at least about 100,000, about 110,000, about 120,000, about 130,000, about 140,000, about 150,000, about 160,000, about 170,000, about 180,000, about 190,000, about 200,000, about 210,000, about 220,000, about 230,000, about 240,000, about 250,000, about 260,000, about 270,000, about 280,000, about 290,000, about or 300,000 vials of passage 4 (P4) cells. In some embodiments, each vial of P4 cells comprises about 10 million, about 20 million, about 30 million, about 40 million, about 50 million, about 60 million, about 70 million, about 80 million, about 90 million, about 100 million, about 110 million, about 120 million, about 130 million, about 140, 150 million, about 160 million, about 170 million, about 180 million, about 290 million, about 200 million or more than 200 million cells. In some embodiments, each vial of P4 cells comprises at least about 10 million, about 20 million, about 30 million, about 40 million, about 50 million, about 60 million, about 70 million, about 80 million, about 90 million, about 100 million, about 110 million, about 120 million, about 130 million, about 140, 150 million, about 160 million, about 170 million, about 180 million, about 290 million, about 200 million or more than 200 million cells. In some embodiments, this allows the treatment of about 100,000, about 110,000, about 120,000, about 130,000, about 140,000, about 150,000, about 160,000, about 170,000, about 180,000, about 190,000, about 200,000, about 210,000, about 220,000, about 230,000, about 240,000, about 250,000, about 260,000, about 270,000, about 280,000, about 290,000, about or 300,000 patients. In some embodiments, this allows the treatment of at least about 100,000, about 110,000, about 120,000, about 130,000, about 140,000, about 150,000, about 160,000, about 170,000, about 180,000, about 190,000, about 200,000, about 210,000, about 220,000, about 230,000, about 240,000, about 250,000, about 260,000, about 270,000, about 280,000, about 290,000, about or 300,000 patients.

### Cryorecovery

The cryopreserved MSCs may be thawed and undergo a second expansion. In some embodiments, MSCs and compositions comprising the same are provided to an end user (e.g., treatment facility) in a condition where they can be immediately be used (i.e., injected into a subject) and with minimal processing. In practice, facilities where the use of MSCs would take place likely do not have personnel trained in MSC sample prep. Therefore, there is a need for methods and systems of MSC sample preparation for immediate injection by the end user and where the MSCs do not require further processing steps upon arrival to the treatment facility.

The methods and systems disclosed herein comprise sample preparation of MSCs to including thawing cryopreserved MSCs and maintaining the MSCs under specific conditions (e.g. specific temperature(s)) for a period of time. The methods and systems disclosed herein also comprise sample preparation of MSCs including thawing cryopreserved MSCs, maintaining the MSCs for a period of time under specific conditions (e.g. a specific/first temperature), and then maintaining the MSCs under different conditions (e.g. at a different temperature than the temperature under which the MSCs were maintained immediately post-thaw). In some embodiments, this change in temperature is a cooling. In some embodiments, the MSCs are maintained in hypothermic conditions post-thaw until direct infusion into a subject.

Provided herein, in some embodiments, is a method of warming a cryopreserved population of stem cells to a first temperature and storing said stem cells at a second temperature less than about 40° C. In some embodiments, the first temperature that the cryopreserved population of stem cells is warmed to is greater than about 0° C. In some embodiments, the first temperature is greater than about 20° C. In some embodiments, the second temperature that the stem cells are stored at is a hypothermic temperature.

In some embodiments, a frozen cryovial comprising cryopreserved cells (e.g., P3 cells) are placed into a 37° C. water bath. The vial will be kept in the water bath until approximately 80% of ice has melted (which takes about two to three minutes). A cryovial is then be sprayed with sterile 70% ethanol (EtOH), wiped with sterile wipes and transferred into a biosafety cabinet. Cells in the thawed vial can then transferred to a sterile conical tube, the cells diluted with PlasmaLyte-A+0.5% HAS, centrifuged (e.g., at room temperature) to form a cell pellet, and the supernatant is removed and the pellet is resuspended in a culturing medium, e.g., MSC Culture Media, for further plating and culturing, e.g., as Passage 4 (P4).

Provided herein, in one aspect, is a method for preparing stem cells for infusion, the method comprising: (a) providing a cryopreserved population of cells comprising said stem cells; (b) warming said stem cells to a first temperature and holding said stem cells at said first temperature for a first period of time; and (c) changing said first temperature to a second temperature and maintaining said stem cells at said second temperature for a second period of time.

In some embodiments, said first temperature is greater than about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C. In some embodiments, said first temperature is about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C. In some embodiments, said first temperature is greater than 0° C.

In some embodiments, said second temperature is less than about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C. In some embodiments, said second temperature is about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C. In some embodiments, said second temperature is hypothermic. In some embodiments, said second temperature is less than 40° C.

In some embodiments, said first time period is less than about one week. In some embodiments, said time period is less than about 7 days, about 6 days, about 5 days, about 4 days, about 3 days, about 2 days, or about 1 day. In some embodiments, said time period is less than about 5 days. In some embodiments, said time period is less than about 2 days. In some embodiments, said time period is less than about 1 day. In some embodiments, said time period is less than about 24 hours, about 23 hours, about 22 hours, about 21 hours, about 20 hours, about 19 hours, about 18 hours, about 17 hours, about 16 hours, about 15 hours, about 14 hours, about 13 hours, about 12 hours, about 11 hours, about 10 hours, about 9 hours, about 8 hours, about 7 hours, about 6 hours, about 5 hours, about 4 hours, about 3 hours, about 2 hours, or about 1 hour. In some embodiments, said time period is less than about 12 hours. In some embodiments, said time period is less than about 6 hours. In some embodiments, said time period is less than about 2 hours. In some embodiments, said time period is less than about 60 minutes, about 59 minutes, about 58 minutes, about 57 minutes, about 56 minutes, about 55 minutes, about 54 minutes, about 53 minutes, about 52 minutes, about 51 minutes, about 50 minutes, about 49 minutes, about 48 minutes, about 47 minutes, about 46 minutes, about 45 minutes, about 44 minutes, about 43 minutes, about 42 minutes, about 41 minutes, about 40 minutes, about 39 minutes, about 38 minutes, about 37 minutes, about 36 minutes, about 35 minutes, about 34 minutes, about 33 minutes, about 32 minutes, about 31 minutes, about 30 minutes, about 29 minutes, about 28 minutes, about 27 minutes, about 26 minutes, about 25 minutes, about 24 minutes, about 23 minutes, about 22 minutes, about 21 minutes, about 20 minutes, about 19 minutes, about 18 minutes, about 17 minutes, about 16 minutes, about 15 minutes, about 14 minutes, about 13 minutes, about 12 minutes, about 11 minutes, about 10 minutes, about 9 minutes, about 8 minutes, about 7 minutes, about 6 minutes, about 5 minutes, about 4 minutes, about 3 minutes, about 2 minutes, or about 1 minute.

In some embodiments, the MSCs are maintained after they are warmed/thawed. In some embodiments, this maintenance is not accounted for in the first period of time described herein. In some embodiments, this maintenance is accounted for in the first period of time. In some embodiments, this maintenance follows similar methods as the MSC culturing methods described herein. In some embodiments, the post-thaw maintenance methods comprise not allowing the MSCs to double in population. In some embodiments, the MSCs are recovered and/or packaged prior to doubling. In some embodiments, the post-thaw culturing maintenance methods comprise culturing plating said stem cells at about 500 cells/cm² to about 4,000 cells/cm². In some embodiments, the post-thaw culturing maintenance methods comprise culturing plating said stem cells at about 500 cells/cm² to about 1,000 cells/cm², about 500 cells/cm² to about 1,500 cells/cm², about 500 cells/cm² to about 2,000 cells/cm², about 500 cells/cm² to about 2,500 cells/cm², about 500 cells/cm² to about 3,000 cells/cm², about 500 cells/cm² to about 3,500 cells/cm², about 500 cells/cm² to about 4,000 cells/cm², about 1,000 cells/cm² to about 1,500 cells/cm², about 1,000 cells/cm² to about 2,000 cells/cm², about 1,000 cells/cm² to about 2,500 cells/cm², about 1,000 cells/cm² to about 3,000 cells/cm², about 1,000 cells/cm² to about 3,500 cells/cm², about 1,000 cells/cm² to about 4,000 cells/cm², about 1,500 cells/cm² to about 2,000 cells/cm², about 1,500 cells/cm² to about 2,500 cells/cm², about 1,500 cells/cm² to about 3,000 cells/cm², about 1,500 cells/cm² to about 3,500 cells/cm², about 1,500 cells/cm² to about 4,000 cells/cm², about 2,000 cells/cm² to about 2,500 cells/cm², about 2,000 cells/cm² to about 3,000 cells/cm², about 2,000 cells/cm² to about 3,500 cells/cm², about 2,000 cells/cm² to about 4,000 cells/cm², about 2,500 cells/cm² to about 3,000 cells/cm², about 2,500 cells/cm² to about 3,500 cells/cm², about 2,500 cells/cm² to about 4,000 cells/cm², about 3,000 cells/cm² to about 3,500 cells/cm², about 3,000 cells/cm² to about 4,000 cells/cm², or about 3,500 cells/cm² to about 4,000 cells/cm². In some embodiments, the post-thaw culturing maintenance methods comprise culturing plating said stem cells at about 500 cells/cm², about 1,000 cells/cm², about 1,500 cells/cm², about 2,000 cells/cm², about 2,500 cells/cm², about 3,000 cells/cm², about 3,500 cells/cm², or about 4,000 cells/cm². In some embodiments, the post-thaw culturing maintenance methods comprise culturing plating said stem cells at least about 500 cells/cm², about 1,000 cells/cm², about 1,500 cells/cm², about 2,000 cells/cm², about 2,500 cells/cm², about 3,000 cells/cm², or about 3,500 cells/cm². In some embodiments, the post-thaw culturing maintenance methods comprise culturing plating said stem cells at most about 1,000 cells/cm², about 1,500 cells/cm², about 2,000 cells/cm², about 2,500 cells/cm², about 3,000 cells/cm², about 3,500 cells/cm², or about 4,000 cells/cm². In some embodiments, the post-thaw maintenance methods comprise plating said stem cells at about 3,000 to about 10,000, about 3,000 to about 9,000, about 3,000 to about 8,000 about, about 3,000 to about 7,000, about 3,000 to about 6,000, about 3,000 to about 5,000, about 3,000 to about 4,000, about 10,000 to about 50,000, about 10,000 to about 40,000, about 10,000 to about 30,000, about 10,000 to about 20,000, about 20,000 to about 50,000, about 20,000 to about 40,000, about 20,000 to about 30,000, about 30,000 to about 50,000, about 30,000 to about 40,000, or about 40,000 to about 50,000 cells/cm² at said first temperature. In some embodiments, for a method provided herein, the method further comprises, prior to (c), maintaining said stem cells at about 3,000 cells/cm² to about 50,000 cells/cm² at said first temperature. In some embodiments, the post-thaw culturing maintenance methods comprise culturing plating said stem cells at about 10,000 cells/cm² to about 50,000 cells/cm². In some embodiments, the post-thaw culturing maintenance methods comprise culturing plating said stem cells at about 10,000 cells/cm² to about 15,000 cells/cm², about 10,000 cells/cm² to about 20,000 cells/cm², about 10,000 cells/cm² to about 25,000 cells/cm², about 10,000 cells/cm² to about 30,000 cells/cm², about 10,000 cells/cm² to about 35,000 cells/cm², about 10,000 cells/cm² to about 40,000 cells/cm², about 10,000 cells/cm² to about 45,000 cells/cm², about 10,000 cells/cm² to about 50,000 cells/cm², about 15,000 cells/cm² to about 20,000 cells/cm², about 15,000 cells/cm² to about 25,000 cells/cm², about 15,000 cells/cm² to about 30,000 cells/cm², about 15,000 cells/cm² to about 35,000 cells/cm², about 15,000 cells/cm² to about 40,000 cells/cm², about 15,000 cells/cm² to about 45,000 cells/cm², about 15,000 cells/cm² to about 50,000 cells/cm², about 20,000 cells/cm² to about 25,000 cells/cm², about 20,000 cells/cm² to about 30,000 cells/cm², about 20,000 cells/cm² to about 35,000 cells/cm², about 20,000 cells/cm² to about 40,000 cells/cm², about 20,000 cells/cm² to about 45,000 cells/cm², about 20,000 cells/cm² to about 50,000 cells/cm², about 25,000 cells/cm² to about 30,000 cells/cm², about 25,000 cells/cm² to about 35,000 cells/cm², about 25,000 cells/cm² to about 40,000 cells/cm², about 25,000 cells/cm² to about 45,000 cells/cm², about 25,000 cells/cm² to about 50,000 cells/cm², about 30,000 cells/cm² to about 35,000 cells/cm², about 30,000 cells/cm² to about 40,000 cells/cm², about 30,000 cells/cm² to about 45,000 cells/cm², about 30,000 cells/cm² to about 50,000 cells/cm², about 35,000 cells/cm² to about 40,000 cells/cm², about 35,000 cells/cm² to about 45,000 cells/cm², about 35,000 cells/cm² to about 50,000 cells/cm², about 40,000 cells/cm² to about 45,000 cells/cm², about 40,000 cells/cm² to about 50,000 cells/cm², or about 45,000 cells/cm² to about 50,000 cells/cm². In some embodiments, the post-thaw culturing maintenance methods comprise culturing plating said stem cells at about 10,000 cells/cm², about 15,000 cells/cm², about 20,000 cells/cm², about 25,000 cells/cm², about 30,000 cells/cm², about 35,000 cells/cm², about 40,000 cells/cm², about 45,000 cells/cm², or about 50,000 cells/cm². In some embodiments, the post-thaw culturing maintenance methods comprise culturing plating said stem cells at least about 10,000 cells/cm², about 15,000 cells/cm², about 20,000 cells/cm², about 25,000 cells/cm², about 30,000 cells/cm², about 35,000 cells/cm², about 40,000 cells/cm², or about 45,000 cells/cm². In some embodiments, the post-thaw culturing maintenance methods comprise culturing plating said stem cells at most about 15,000 cells/cm², about 20,000 cells/cm², about 25,000 cells/cm², about 30,000 cells/cm², about 35,000 cells/cm², about 40,000 cells/cm², about 45,000 cells/cm², or about 50,000 cells/cm². In some embodiments, for a method provided herein, the method further comprises, prior to (c), maintaining said stem cells at about 3,000, about 4,000, about 5,000, about 6,000, about 7,000, about 8,000, about 9,000, 10,000, about 20,000, about 30,000, about 40,000, or about 50,000 cells/cm² at said first temperature. In some embodiments, for a method provided herein, the method further comprises, prior to (c), maintaining said stem cells at about 32,000 cells/cm² at said first temperature. In some embodiments, the post-thaw maintaining takes place in a T25 flask. In some embodiments, the post-thaw maintaining takes place in a T75 flask. In some embodiments, the post-thaw maintaining takes place in another sized flasks.

In some embodiments, the post-thaw culturing maintenance methods increases the number of cells in a culture vessel from about 5% to about 500%. As examples, the post-thaw culturing maintenance methods may increase the number of cells in a culture vessel by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 120%, about 140%, about 160%, about 180%, about 200%, about 220%, about 240%, about 260%, about 280%, about 300%, about 320%, about 340%, about 360%, about 380%, about 400%, about 420%, about 440%, about 460%, about 480%, or about 500%. The post-thaw culturing maintenance methods may increase the number of cells in a culture vessel by about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 100%, about 100% to about 200%, about 200% to about 300%, about 300% to about 400%, or about 400% to about 500%.

In some embodiments, the stem cells are packaged in 5 mL volumes of 10×10⁶ live cells/ml in Plasma-Lyte A+2.5% HSA (Rinse Media). In some embodiments, the stem cells are packaged in volumes of about 1×10⁶ live cells/ml to about 12×10⁶ live cells/ml. In some embodiments, the stem cells are packaged in volumes of about 1×10⁶ live cells/ml to about 2×10⁶ live cells/ml, about 1×10⁶ live cells/ml to about 3×10⁶ live cells/ml, about 1×10⁶ live cells/ml to about 4×10⁶ live cells/ml, about 1×10⁶ live cells/ml to about 5×10⁶ live cells/ml, about 1×10⁶ live cells/ml to about 6×10⁶ live cells/ml, about 1×10⁶ live cells/ml to about 7×10⁶ live cells/ml, about 1×10⁶ live cells/ml to about 8×10⁶ live cells/ml, about 1×10⁶ live cells/ml to about 9×10⁶ live cells/ml, about 1×10⁶ live cells/ml to about 10×10⁶ live cells/ml, about 1×10⁶ live cells/ml to about 11×10⁶ live cells/ml, about 1×10⁶ live cells/ml to about 12×10⁶ live cells/ml, about 2×10⁶ live cells/ml to about 3×10⁶ live cells/ml, about 2×10⁶ live cells/ml to about 4×10⁶ live cells/ml, about 2×10⁶ live cells/ml to about 5×10⁶ live cells/ml, about 2×10⁶ live cells/ml to about 6×10⁶ live cells/ml, about 2×10⁶ live cells/ml to about 7×10⁶ live cells/ml, about 2×10⁶ live cells/ml to about 8×10⁶ live cells/ml, about 2×10⁶ live cells/ml to about 9×10⁶ live cells/ml, about 2×10⁶ live cells/ml to about 10×10⁶ live cells/ml, about 2×10⁶ live cells/nil to about 11×10⁶ live cells/ml, about 2×10⁶ live cells/ml to about 12×10⁶ live cells/ml, about 3×10⁶ live cells/ml to about 4×10⁶ live cells/ml, about 3×10⁶ live cells/ml to about 5×10⁶ live cells/ml, about 3×10⁶ live cells/ml to about 6×10⁶ live cells/ml, about 3×10⁶ live cells/ml to about 7×10⁶ live cells/ml, about 3×10⁶ live cells/ml to about 8×10⁶ live cells/ml, about 3×10⁶ live cells/ml to about 9×10⁶ live cells/ml, about 3×10⁶ live cells/ml to about 10×10⁶ live cells/ml, about 3×10⁶ live cells/ml to about 11×10⁶ live cells/ml, about 3×10⁶ live cells/ml to about 12×10⁶ live cells/ml, about 4×10⁶ live cells/ml to about 5×10⁶ live cells/ml, about 4×10⁶ live cells/ml to about 6×10⁶ live cells/ml, about 4×10⁶ live cells/ml to about 7×10⁶ live cells/ml, about 4×10⁶ live cells/ml to about 8×10⁶ live cells/ml, about 4×10⁶ live cells/nil to about 9×10⁶ live cells/ml, about 4×10⁶ live cells/ml to about 10×10⁶ live cells/ml, about 4×10⁶ live cells/ml to about 11×10⁶ live cells/ml, about 4×10⁶ live cells/ml to about 12×10⁶ live cells/ml, about 5×10⁶ live cells/ml to about 6×10⁶ live cells/ml, about 5×10⁶ live cells/nil to about 7×10⁶ live cells/ml, about 5×10⁶ live cells/ml to about 8×10⁶ live cells/ml, about 5×10⁶ live cells/nil to about 9×10⁶ live cells/ml, about 5×10⁶ live cells/ml to about 10×10⁶ live cells/ml, about 5×10⁶ live cells/ml to about 11×10⁶ live cells/ml, about 5×10⁶ live cells/nil to about 12×10⁶ live cells/ml, about 6×10⁶ live cells/ml to about 7×10⁶ live cells/ml, about 6×10⁶ live cells/ml to about 8×10⁶ live cells/ml, about 6×10⁶ live cells/ml to about 9×10⁶ live cells/ml, about 6×10⁶ live cells/ml to about 10×10⁶ live cells/ml, about 6×10⁶ live cells/ml to about 11×10⁶ live cells/ml, about 6×10⁶ live cells/ml to about 12×10⁶ live cells/ml, about 7×10⁶ live cells/ml to about 8×10⁶ live cells/ml, about 7×10⁶ live cells/ml to about 9×10⁶ live cells/ml, about 7×10⁶ live cells/ml to about 10×10⁶ live cells/ml, about 7×10⁶ live cells/nil to about 11×10⁶ live cells/ml, about 7×10⁶ live cells/ml to about 12×10⁶ live cells/ml, about 8×10⁶ live cells/ml to about 9×10⁶ live cells/ml, about 8×10⁶ live cells/ml to about 10×10⁶ live cells/ml, about 8×10⁶ live cells/ml to about 11×10⁶ live cells/ml, about 8×10⁶ live cells/nil to about 12×10⁶ live cells/ml, about 9×10⁶ live cells/ml to about 10×10⁶ live cells/ml, about 9×10⁶ live cells/ml to about 11×10⁶ live cells/ml, about 9×10⁶ live cells/ml to about 12×10⁶ live cells/ml, about 10×10⁶ live cells/ml to about 11×10⁶ live cells/ml, about 10×10⁶ live cells/ml to about 12×10⁶ live cells/ml, or about 11×10⁶ live cells/ml to about 12×10⁶ live cells/ml. In some embodiments, the stem cells are packaged in volumes of about 1×10⁶ live cells/ml, about 2×10⁶ live cells/ml, about 3×10⁶ live cells/ml, about 4×10⁶ live cells/ml, about 5×10⁶ live cells/ml, about 6×10⁶ live cells/ml, about 7×10⁶ live cells/ml, about 8×10⁶ live cells/ml, about 9×10⁶ live cells/ml, about 10×10⁶ live cells/ml, about 11×10⁶ live cells/ml, or about 12×10⁶ live cells/ml. In some embodiments, the stem cells are packaged in volumes of at least about 1×10⁶ live cells/ml, about 2×10⁶ live cells/ml, about 3×10⁶ live cells/ml, about 4×10⁶ live cells/ml, about 5×10⁶ live cells/ml, about 6×10⁶ live cells/ml, about 7×10⁶ live cells/ml, about 8×10⁶ live cells/ml, about 9×10⁶ live cells/ml, about 10×10⁶ live cells/ml, or about 11×10⁶ live cells/ml. In some embodiments, the stem cells are packaged in volumes of at most about 2×10⁶ live cells/ml, about 3×10⁶ live cells/ml, about 4×10⁶ live cells/ml, about 5×10⁶ live cells/ml, about 6×10⁶ live cells/ml, about 7×10⁶ live cells/ml, about 8×10⁶ live cells/ml, about 9×10⁶ live cells/ml, about 10×10⁶ live cells/ml, about 11×10⁶ live cells/ml, or about 12×10⁶ live cells/ml.

Once the stem cells are packaged, the stem cells are put under the second temperature for the second time period. In some embodiments, the second temperature is less than about 40° C., about 39° C., about 38° C., about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., about 21° C., about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., or about 1° C. In some embodiments, said cell culture is maintained at about 40° C., about 39° C., about 38° C., about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., about 21° C., about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., or about 1° C. In some embodiments, the second temperature is less than 37° C. In some embodiments, the second temperature is less than 35° C. In some embodiments, the second temperature is less than 30° C. In some embodiments, the second temperature is less than 25° C. In some embodiments, said the second temperature is less than 20° C. In some embodiments, the second temperature is about 2° C. to about 8° C.

In some embodiments, said second time period is less than about one week. In some embodiments, said time period is less than about 7 days, about 6 days, about 5 days, about 4 days, about 3 days, about 2 days, or about 1 day. In some embodiments, said time period is less than about 5 days. In some embodiments, said time period is less than about 2 days. In some embodiments, said time period is less than about 1 day. In some embodiments, said time period is less than about 24 hours, about 23 hours, about 22 hours, about 21 hours, about 20 hours, about 19 hours, about 18 hours, about 17 hours, about 16 hours, about 15 hours, about 14 hours, about 13 hours, about 12 hours, about 11 hours, about 10 hours, about 9 hours, about 8 hours, about 7 hours, about 6 hours, about 5 hours, about 4 hours, about 3 hours, about 2 hours, or about 1 hour. In some embodiments, said time period is less than about 12 hours. In some embodiments, said time period is less than about 6 hours. In some embodiments, said time period is less than about 2 hours. In some embodiments, said time period is less than about 60 minutes, about 59 minutes, about 58 minutes, about 57 minutes, about 56 minutes, about 55 minutes, about 54 minutes, about 53 minutes, about 52 minutes, about 51 minutes, about 50 minutes, about 49 minutes, about 48 minutes, about 47 minutes, about 46 minutes, about 45 minutes, about 44 minutes, about 43 minutes, about 42 minutes, about 41 minutes, about 40 minutes, about 39 minutes, about 38 minutes, about 37 minutes, about 36 minutes, about 35 minutes, about 34 minutes, about 33 minutes, about 32 minutes, about 31 minutes, about 30 minutes, about 29 minutes, about 28 minutes, about 27 minutes, about 26 minutes, about 25 minutes, about 24 minutes, about 23 minutes, about 22 minutes, about 21 minutes, about 20 minutes, about 19 minutes, about 18 minutes, about 17 minutes, about 16 minutes, about 15 minutes, about 14 minutes, about 13 minutes, about 12 minutes, about 11 minutes, about 10 minutes, about 9 minutes, about 8 minutes, about 7 minutes, about 6 minutes, about 5 minutes, about 4 minutes, about 3 minutes, about 2 minutes, or about 1 minute.

### Methods of Manufacturing γMSCs

The present invention provides methods for manufacturing γMSCs, the method comprising obtaining a culture of MSCs and contacting the MSCs with IFNγ.

The MSCs may comprise vertebral bone marrow MSCs (vBM-MSCs), vertebral bone-adherent MSCs (vBA-MSCs), or both. The MSCs may be obtained from one or more vertebral bodies, and the vertebral bodies may be deceased humans.

MSCs may undergo a second expansion after cryopreservation. The second expansion may occur similarly to a first primary expansion, as described herein. The second expansion may also include additional components in the expansion medium. For example, the culture medium of the second expansion may comprise IFNγ. The IFNγ may be present in the expansion medium in order to prime the MSCs. Priming the MSCs with IFNγ creates γMSCs which may be used in human administration for the treatment of GVHD.

In some embodiments, P3 cells that have been cryofrozen are thawed or P3 that are detached from their culturing vessels are then further plated and cultured to form the Passage 4 (P4) cells. See, e.g., FIG. 1A to FIG. 1D.

P3 cells can be transferred to a new, sterile centrifuge tube and the cells may be diluted with about 2 volumes of PlasmaLyte-A+0.5% HSA. The cells can be then centrifuged at about 500 RCF for 10 minutes at room temperature to form a cell pellet. The supernatant is removed and the pellet is resuspended in a culturing medium, e.g., MSC Culture Media as described herein. Cell viability and count can be determined using Trypan Blue Exclusion via Manual Counting (or using AO/PI Method via automatic Cellometer). MSCs can be seeded at about 3,000-4,000 cells/cm2 onto eight CellBIND® 5-chamber CellSTACKs® and placed into a 5% CO₂, 37° C., humidified tissue culture incubator. The plating step will be considered Day 0 and this cell culture step is considered Passage 4 (P4).

At Day 3, the MSC culture undergoes a media change to remove expended media and non-adherent cells. MSCs are then returned to the tissue culture incubator for two additional days. At Day 5, half of the media from a flask is removed and a sample for BAcT/Alert (according to according to 21 CFR 610.12) is collected from this expended media from each flask. An aliquot (of about half of expended media that was removed) of culture media+50 ng/ml γ-IFN (R&D Systems, Minneapolis, USA) is made and added to each flask. The interferon gamma primed P4 cells are once again returned to the tissue culture incubator for two additional days. At Day 7, γ-MSCs cell density and morphology are inspected using a bright-field inverted microscope. γ-MSCs are harvested from culture via TrypLE™ Select, washed three times with phosphate buffered saline (PBS), and assessed for cell viability and count. Final product must meet release criteria as defined in Table 5.

Table 5 lists testing performed on the P4 cells and preferable features that γ-MSCs may possess.

TABLE 5
P4 γ-MSC Characterization

| Attribute | Test | Preferable Features |
|---|---|---|
| Expended Media | BacT/Alert according to 21 CFR | No Growth |
| Sterility from Day 5 | 610.12 | |
| Media Change | | |
| Viability and Cell | AO/PI via Cellometer or Trypan | 85 ± 15%, and sufficient |
| Count | Blue exclusion via Manual Count | number of cells for dose |
| | | (based on patient's ideal |
| | | body weight) |
| Rapid Final Cell | Gram Stain (Microscopy) | No organisms seen |
| Product Sterility | | |
| Mycoplasma | Lonza MycoAlert | Negative |
| Endotoxin | Charles River Endosafe ® PTS | <2.5 EU/ml |
| Final Cell Product | Flow Cytometry | CD105 positive (≥85%), |
| Identity | | CD45 negative (≤3%) |
| Final Cell Product | BacT/Alert according to 21 CFR | No Growth [will report |
| Sterility | 610.12 | after 14-days] |

The IFNγ may be present in the expansion medium at a concentration of about 100 U/ml to about 1000 U/ml. The IFNγ may be present in the expansion medium at a concentration of about 10 U/ml to about 500 U/ml, about 10 U/ml to about 1000 U/ml, about 10 U/ml to about 1500 U/ml, about 10 U/ml to about 2000 U/ml, about 50 U/ml to about 500 U/ml, about 50 U/ml to about 1000 U/ml, about 50 U/ml to about 1500 U/ml, about 50 U/ml to about 2000 U/ml, about 100 U/ml to about 500 U/ml, about 100 U/ml to about 1500 U/ml, and about 100 U/ml to about 2000 U/ml. The IFNγ may be present in the expansion medium at a concentration of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, or 2000 U/ml.

The IFNγ may be present in the expansion medium at a concentration of about 1 to about 30 ng/ml. The IFNγ may be present in the expansion medium at a concentration of about 1 to about 15 ng/ml, 1 to about 45 ng/ml, or 1 to about 60 ng/ml. The IFNγ may be present in the expansion medium at a concentration of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 ng/ml.

The second expansion may occur for about 7 days. The second expansion may occur for about 1 to about 3 days, about 1 to about 5 days, about 1 to about 9 days, about 1 to about 11 days, about 1 to about 13 days, or about 1 to about 15 days. The second expansion may occur for about 1, 2, 3, 4, 5, 6, 7, 8, 9, 10, 11, 12, 13, 14, or 15 days.

The IFNγ may be added to the expansion media during the final day, the final 2 days, or the final 3 days of the second expansion. The IFNγ may also be added to the expansion media during the final 4 days, the final 5 days, the final 6 days, the final 7 days, the final 8 days, the final 9 days, the final 10 days, the final 11 days, the final 12 days, the final 13 days, or the final 14 days of the second expansion.

In some cases, when the MSCs were being interferon γ-primed, the culture was not in a hypoxic condition. As examples, the culture did not have decreased levels of O₂, did not have increased levels of CO₂, and/or lacked the presence of a hypoxia mimetic. Decreased levels of O₂ may be from about 1% O₂ to about 5% O₂. Increased levels of CO₂ can be about 5% CO₂ or higher. Illustrative hypoxia mimetics include desferoxamine, cobalt chloride, hydralazine, nickel chloride, diazoxide, and dimethyloxalyglycine.

In embodiments, a clinical product comprising γ-IFN primed MSCs substantially lacks any residual γ-IFN. By substantially lacking residual γ-IFN can mean that an ELISA in unable to detect the cytokine in a wash media nor in a sample of the cell product itself. If any residual γ-IFN is detected in a clinical product the cells may be pelleted, washed and resuspended until γ-IFN becomes undetectable. By administering a clinical product that substantially lacks γ-IFN reduces the likelihood that a subject incur a γ-IFN mediated toxicity (Table 5).

In some embodiments, the preparations and compositions of the present disclosure comprise interferon γ-primed human mesenchymal stromal cells (γMSCs) for preventing or reducing the likelihood of Graft Versus Host Disease (GVHD) or a symptom thereof in a human subject that has been administered a hematopoietic stem cell transplant (HCT). The MSCs may be obtained from one deceased human, as described herein. The MSCs may also be obtained from one or more vertebral bodies. The MSCs may comprise vertebral bone marrow MSCs (vBM-MSCs), vertebral bone-adherent MSCs (vBA-MSCs), or both. The vBM-MSCs and vBA-MSCs may be derived as is described herein.

In embodiments, Endotoxin testing is performed using the Endosafe®-PTS™ system according to SOP EPIC Limulus Amebocyte Lysate (LAL) Assay for Endotoxin Detection Using the Endosafe®-PTS™. The Endosafe®-PTS™ is a rapid, point-of-use test system that provides quantitative LAL results within 15 minutes after specimen preparation. The PTS™ utilizes LAL reagents in an FDA-licensed disposable test cartridge with a handheld reader for a completely contained, real-time endotoxin testing system. The PTS™ can be used to get a quick read on raw materials and STAT samples that require immediate analysis. The flexibility of the PTS™ allows it to be used in conventional, quality control testing laboratories as well as at the point of sample collection. Preferably cells for clinical use have <2.5 EU/ml for release.

As shown in FIG. 1A, cultured P3 cells are shipped to a treatment site. The P3 cells may be cryopreserved and shipped accordingly. However, if the treatment site is local or within the same facility, campus, or healthcare complex, the P3 cells may not need to be cryopreserved and are instead shipped on ice, at room temperature, or at a culturing temperature (e.g., about 37° C.). The P3 cells are warmed (when cryopreserved or when shipped cold) or otherwise prepared for further culturing and are cultured to obtain P4 cells. The P4 cells are primed with IFNγ as disclosed herein. And, the final γ-MSCs are prepared (e.g., concentrated, diluted, and/or combined with pharmaceutically-acceptable solvents or carriers and the like) for transplant into a patient in need thereof.

As shown in FIG. 1B, cultured P4 cells are primed with IFNγ as disclosed herein. The γ-MSCs are shipped to a treatment site. The γ-MSCs are may be cryopreserved and shipped accordingly. However, if the treatment site is local or within the same facility, campus, or healthcare complex, the γ-MSCs are may not need to be cryopreserved and are instead shipped on ice, at room temperature, or at another temperature (e.g., about 37° C.). The γ-MSCs are warmed (when cryopreserved or when shipped cold) and/or otherwise prepared (e.g., concentrated, diluted, and/or combined with pharmaceutically-acceptable solvents or carriers and the like) for transplant to a patient in need thereof.

As shown in FIG. 1C, cultured P3 cells are primed with IFNγ as disclosed herein. The γ-MSCs are shipped to a treatment site. The γ-MSCs may be cryopreserved and shipped accordingly. However, if the treatment site is local or within the same facility, campus, or healthcare complex, the γ-MSCs may not need to be cryopreserved and are instead shipped on ice, at room temperature, or at a culturing temperature (e.g., about 37° C.). The γ-MSCs are warmed (when cryopreserved or when shipped cold) or otherwise prepared for further culturing and are cultured to obtain P4-stage γ-MSCs. The P4-stage γ-MSCs are prepared (e.g., concentrated, diluted, and/or combined with pharmaceutically-acceptable solvents or carriers and the like) for transplant to a patient in need thereof.

As shown in FIG. 1D, cultured P4 cells are shipped to a treatment site. The P4 cells may be cryopreserved and shipped accordingly. However, if the treatment site is local or within the same facility, campus, or healthcare complex, the P4 cells may not need to be cryopreserved and are instead shipped on ice, at room temperature, or at a culturing temperature (e.g., about 37° C.). The P4 cells are warmed (when cryopreserved or when shipped cold) or otherwise prepared for priming with IFNγ as disclosed herein. And, the final γ-MSCs are prepared (e.g., concentrated, diluted, and/or combined with pharmaceutically-acceptable solvents or carriers and the like) for transplant to a patient in need thereof.

In any of the herein disclosed aspects or embodiments, rather than cryopreserving MSCs or γMSCs and thawing the MSCs (which are subsequently primed) or thawing the γMSCs prior to use (either immediately or after one or more culturing steps), fresh MSCs or fresh γMSCs may be used.

Alternates and/or Supplements to Priming MSCs with IFNγ

In vivo, MSCs are stimulated in response to abnormal conditions, e.g., caused by infection, cancer, and injury. The presence of circulating IFNγ can be a signal to MSCs which identifies an abnormal condition. In response to the circulating IFNγ, the MSCs are primed and secrete, at least, factors and proteins that help remedy the abnormal condition. In various aspects of the present disclosure, MSCs are primed in vitro or ex vivo by contact with IFNγ to transform the cells into γMSCs. In some cases, MSCs are only primed by contact with IFNγ. In other cases, MSCs are primed by contact with IFNγ and also stimulated with one or both of serum starvation and temperature shock. In yet other cases, MSCs are not primed by contact with IFNγ and are instead stimulated with one or both of serum starvation and temperature shock.

Serum starvation is the removal of serum (which is a source of proteins, including growth factors) from a culturing medium. Serum starvation, like the presence of IFNγ, informs the MSCs of an abnormal condition, which likewise will stimulate the MSCs. Serum starvation can occur contemporaneously with the IFNγ priming (as disclosed elsewhere herein). In certain methods, human platelet lysate (hPL) is omitted from the culturing medium to enact serum starvation. As examples, the serum starvation may occur during the final day or the final two days of culturing. In some cases, MSCs are primed by contact with IFNγ and are stimulated by serum starvation.

In some embodiments, MSCs are not primed by contact with IFNγ and are instead stimulated by serum starvation.

Temperature shock is an increase in the temperature of a cell culture to a degree and duration that the cells are shocked, e.g., induce expression of heat shock proteins. Temperature shock is another indicator of an abnormal condition (e.g., suggesting an infection) which simulates MSCs. In some embodiments, the temperature of the cell culture is raised to about 40° C. or higher. The duration may be for less than an hour, for about an hour, for about one or two hours, or for more than two hours. In various embodiments, the temperature shock occurs during the final day of culturing. In some embodiments, the temperature shock occurs in the final hour or final hours of culturing. In some cases, MSCs are primed by contact with IFNγ and are stimulated by temperature shock.

In some embodiments, MSCs are not primed by contact with IFNγ and are instead stimulated by temperature shock.

In various embodiments, MSCs are not primed by contact with IFNγ and are instead stimulated by serum starvation and by temperature shock.

In embodiments, MSCs are primed by contact with IFNγ and stimulated by serum starvation and by temperature shock.

In any herein disclosed method or composition, a γMSC may be an MSC that was only primed by contact with IFNγ. In any herein disclosed method or composition, a γMSC may be an MSC that was primed by contact with IFNγ and also stimulated with one or both of serum starvation and temperature shock. In any herein disclosed method or composition, a “γMSC” or “MSC” may be an MSC that was not primed by contact with IFNγ and was instead stimulated with one or both of serum starvation and temperature shock.

Compositions of γMSCs and/or MSCs

A clinical product may be tested analyzed to measure expression values of a subset of genes to determine if γ-IFN priming (as disclosed herein) introduced additional inter-donor variation other than what intrinsically exists in MSCs.

A clinical product may be assessed for the integrity of the chromosomes (karyotype). Twenty cells in metaphase from the standard MSC preparation and γ-IFN-primed MSCs (as disclosed herein) can be examined to detect the presence of 46 chromosomes including two sex chromosomes and any consistent structural or numerical abnormalities.

The surface phenotype of cells in the clinical product can be determined by flow cytometry. γ-IFN-primed MSCs should express HLA Class I and II molecules, as well as PD-L1 and PD-L2, but not CD80 or CD86.

MSCs and γ-MSCs (as disclosed herein) should differentiate in vitro into osteoblasts, adipocytes, and chondroblasts. Clinical products can be tested to determine if they can differentiate in vitro into, at least, these cell types. Differentiation of osteoblasts, adipocytes, and chondroblasts, respectively, can be detected morphologically using Alizarin Red S, Oil Red O, and Alcian Blue histochemical staining.

In some cases, a clinical product (or a previous-stage product) is tested for sterility by Gram stain and the final cellular material using the BacT/Alert system by inoculating aerobic and anaerobic test bottles and incubating for a 14-day culture period. The BacT/Alert uses a constant monitoring system that will alert presence of cellular growth at any point during the 14-day incubation.

The numbers of cells in clinical product and the number of viable cells can be assayed via the nucleic acid binding dyes acridine orange (AO) and propidium iodide (PI) using an automated cell counter (Cellometer), or via trypan blue exclusion method using manual count.

In various embodiment, a clinical product may comprise from about 1×10⁶ γMSCs/ml to about 1×10⁷ γMSCs/ml of infusion medium. As examples, the clinical product may comprise about 1×10⁶ γMSCs/ml, 2×10⁶ γMSCs/ml, 3×10⁶ γMSCs/ml, 4×10⁶ γMSCs/ml, 5×10⁶ γMSCs/ml, 6×10⁶ γMSCs/ml, 7×10⁶ γMSCs/ml, 8×10⁶ γMSCs/ml, 9×10⁶ γMSCs/ml, or 1×10⁷ γMSCs/ml, and any concentation of γMSCs therebetween. In one example, a clinical product comprise about 4×10⁶ γMSCs/ml of infusion media, e.g., about 3.5×10⁶ γMSCs/ml, 3.6×10⁶ γMSCs/ml, 3.7×10⁶ γMSCs/ml, 3.8×10⁶ γMSCs/ml, 3.9×10⁶ γMSCs/ml, 4×10⁶ γMSCs/ml, 4.1×10⁶ γMSCs/ml, 4.2×10⁶ γMSCs/ml, 4.3×10⁶ γMSCs/ml, 4.4×10⁶ γMSCs/ml, or 4.5×10⁶ γMSCs/ml, and any concentation of γMSCs therebetween.

An infusion medium may comprise about 0.5% human serum albumin (HSA).

In various clinical products, no antibiotics or anti-mycotics were used in the MSC manufacturing process, no bovine or porcine components were used in the MSC manufacturing process, and/or irradiation was not used during any step in the MSC manufacturing process.

In some embodiments, the preparations and compositions of the present disclosure may comprise at least 100 million γMSCs and/or MSCs having an antigen profile of more than about 1.75% CD45+ cells, at least about 95% CD105+ cells, and at least about 95% CD166+ cells and the cells may be expanded ex vivo from passage 2 until passage 4 while maintaining population uniformity based upon the antigen profile (i.e. more than about 1.75% CD45+ cells, at least about 95% CD105+ cells, and at least about 95% CD166+ cells).

In some embodiments, the preparations and compositions of the present disclosure may comprise at least 100 million γMSCs and/or MSCs having an antigen profile of less than about 5% CD45+ cells, at least about 95% CD105+ cells, and at least about 95% CD166+ cells and the cells may be expanded ex vivo from passage 2 until passage 4 while maintaining population uniformity based upon the antigen profile (i.e. less than about 1.75% CD45+ cells, at least about 95% CD105+ cells, and at least about 95% CD166+ cells). In embodiments, the γMSCs and/or MSCs have an antigen profile of less than about 4% CD45+ cells, less than about 3% CD45+ cells, less than about 2% CD45+ cells, less than about 1% CD45+ cells, or about 0% CD45+ cells.

In some embodiments, the preparations and compositions of the present disclosure may comprise γMSCs and/or MSCs having an antigen profile of reduced expression of one or more senescent cell markers, as compared to bone marrow-derived MSCs prepared according to known MSC culturing techniques. In some embodiments, the one or more senescent cell markers comprise MIC-A, MIC-B, ULBP2, or any combination thereof. NK cell-mediated immune responses are stimulated by MIC-A, MIC-B, and/or ULBP2.

In some embodiments, the γMSC and/or MSC preparations and compositions described herein comprise a number of cells that express one or more senescent cell markers of about 1% less than bone marrow-derived MSCs to about 100% less than bone marrow-derived MSCs. In some embodiments, the γMSC and/or MSC preparations and compositions described herein comprise an amount of cells that express one or more senescent cell markers of about 100% less than bone marrow-derived MSCs to about 90% less than bone marrow-derived MSCs, about 100% less than bone marrow-derived MSCs to about 80% less than bone marrow-derived MSCs, about 100% less than bone marrow-derived MSCs to about 70% less than bone marrow-derived MSCs, about 100% less than bone marrow-derived MSCs to about 60% less than bone marrow-derived MSCs, about 100% less than bone marrow-derived MSCs to about 50% less than bone marrow-derived MSCs, about 100% less than bone marrow-derived MSCs to about 40% less than bone marrow-derived MSCs, about 100% less than bone marrow-derived MSCs to about 30% less than bone marrow-derived MSCs, about 100% less than bone marrow-derived MSCs to about 20% less than bone marrow-derived MSCs, about 100% less than bone marrow-derived MSCs to about 10% less than bone marrow-derived MSCs, about 100% less than bone marrow-derived MSCs to about 5% less than bone marrow-derived MSCs, about 100% less than bone marrow-derived MSCs to about 1% less than bone marrow-derived MSCs, about 90% less than bone marrow-derived MSCs to about 80% less than bone marrow-derived MSCs, about 90% less than bone marrow-derived MSCs to about 70% less than bone marrow-derived MSCs, about 90% less than bone marrow-derived MSCs to about 60% less than bone marrow-derived MSCs, about 90% less than bone marrow-derived MSCs to about 50% less than bone marrow-derived MSCs, about 90% less than bone marrow-derived MSCs to about 40% less than bone marrow-derived MSCs, about 90% less than bone marrow-derived MSCs to about 30% less than bone marrow-derived MSCs, about 90% less than bone marrow-derived MSCs to about 20% less than bone marrow-derived MSCs, about 90% less than bone marrow-derived MSCs to about 10% less than bone marrow-derived MSCs, about 90% less than bone marrow-derived MSCs to about 5% less than bone marrow-derived MSCs, about 90% less than bone marrow-derived MSCs to about 1% less than bone marrow-derived MSCs, about 80% less than bone marrow-derived MSCs to about 70% less than bone marrow-derived MSCs, about 80% less than bone marrow-derived MSCs to about 60% less than bone marrow-derived MSCs, about 80% less than bone marrow-derived MSCs to about 50% less than bone marrow-derived MSCs, about 80% less than bone marrow-derived MSCs to about 40% less than bone marrow-derived MSCs, about 80% less than bone marrow-derived MSCs to about 30% less than bone marrow-derived MSCs, about 80% less than bone marrow-derived MSCs to about 20% less than bone marrow-derived MSCs, about 80% less than bone marrow-derived MSCs to about 10% less than bone marrow-derived MSCs, about 80% less than bone marrow-derived MSCs to about 5% less than bone marrow-derived MSCs, about 80% less than bone marrow-derived MSCs to about 1% less than bone marrow-derived MSCs, about 70% less than bone marrow-derived MSCs to about 60% less than bone marrow-derived MSCs, about 70% less than bone marrow-derived MSCs to about 50% less than bone marrow-derived MSCs, about 70% less than bone marrow-derived MSCs to about 40% less than bone marrow-derived MSCs, about 70% less than bone marrow-derived MSCs to about 30% less than bone marrow-derived MSCs, about 70% less than bone marrow-derived MSCs to about 20% less than bone marrow-derived MSCs, about 70% less than bone marrow-derived MSCs to about 10% less than bone marrow-derived MSCs, about 70% less than bone marrow-derived MSCs to about 5% less than bone marrow-derived MSCs, about 70% less than bone marrow-derived MSCs to about 1% less than bone marrow-derived MSCs, about 60% less than bone marrow-derived MSCs to about 50% less than bone marrow-derived MSCs, about 60% less than bone marrow-derived MSCs to about 40% less than bone marrow-derived MSCs, about 60% less than bone marrow-derived MSCs to about 30% less than bone marrow-derived MSCs, about 60% less than bone marrow-derived MSCs to about 20% less than bone marrow-derived MSCs, about 60% less than bone marrow-derived MSCs to about 10% less than bone marrow-derived MSCs, about 60% less than bone marrow-derived MSCs to about 5% less than bone marrow-derived MSCs, about 60% less than bone marrow-derived MSCs to about 1% less than bone marrow-derived MSCs, about 50% less than bone marrow-derived MSCs to about 40% less than bone marrow-derived MSCs, about 50% less than bone marrow-derived MSCs to about 30% less than bone marrow-derived MSCs, about 50% less than bone marrow-derived MSCs to about 20% less than bone marrow-derived MSCs, about 50% less than bone marrow-derived MSCs to about 10% less than bone marrow-derived MSCs, about 50% less than bone marrow-derived MSCs to about 5% less than bone marrow-derived MSCs, about 50% less than bone marrow-derived MSCs to about 1% less than bone marrow-derived MSCs, about 40% less than bone marrow-derived MSCs to about 30% less than bone marrow-derived MSCs, about 40% less than bone marrow-derived MSCs to about 20% less than bone marrow-derived MSCs, about 40% less than bone marrow-derived MSCs to about 10% less than bone marrow-derived MSCs, about 40% less than bone marrow-derived MSCs to about 5% less than bone marrow-derived MSCs, about 40% less than bone marrow-derived MSCs to about 1% less than bone marrow-derived MSCs, about 30% less than bone marrow-derived MSCs to about 20% less than bone marrow-derived MSCs, about 30% less than bone marrow-derived MSCs to about 10% less than bone marrow-derived MSCs, about 30% less than bone marrow-derived MSCs to about 5% less than bone marrow-derived MSCs, about 30% less than bone marrow-derived MSCs to about 1% less than bone marrow-derived MSCs, about 20% less than bone marrow-derived MSCs to about 10% less than bone marrow-derived MSCs, about 20% less than bone marrow-derived MSCs to about 5% less than bone marrow-derived MSCs, about 20% less than bone marrow-derived MSCs to about 1% less than bone marrow-derived MSCs, about 10% less than bone marrow-derived MSCs to about 5% less than bone marrow-derived MSCs, about 10% less than bone marrow-derived MSCs to about 1% less than bone marrow-derived MSCs, or about 5% less than bone marrow-derived MSCs to about 1% less than bone marrow-derived MSCs. In some embodiments, the vBA-MSC preparations and compositions described herein comprise an amount of cells that express one or more senescent cell markers of about 100% less than bone marrow-derived MSCs, about 90% less than bone marrow-derived MSCs, about 80% less than bone marrow-derived MSCs, about 70% less than bone marrow-derived MSCs, about 60% less than bone marrow-derived MSCs, about 50% less than bone marrow-derived MSCs, about 40% less than bone marrow-derived MSCs, about 30% less than bone marrow-derived MSCs, about 20% less than bone marrow-derived MSCs, about 10% less than bone marrow-derived MSCs, about 5% less than bone marrow-derived MSCs, or about 1% less than bone marrow-derived MSCs. In some embodiments, the vBA-MSC preparations and compositions described herein comprise an amount of cells that express one or more senescent cell markers of at least about 100% less than bone marrow-derived MSCs, about 90% less than bone marrow-derived MSCs, about 80% less than bone marrow-derived MSCs, about 70% less than bone marrow-derived MSCs, about 60% less than bone marrow-derived MSCs, about 50% less than bone marrow-derived MSCs, about 40% less than bone marrow-derived MSCs, about 30% less than bone marrow-derived MSCs, about 20% less than bone marrow-derived MSCs, about 10% less than bone marrow-derived MSCs, or about 5% less than bone marrow-derived MSCs. In some embodiments, the vBA-MSC preparations and compositions described herein comprise an amount of cells that express one or more senescent cell markers of at most about 90% less than bone marrow-derived MSCs, about 80% less than bone marrow-derived MSCs, about 70% less than bone marrow-derived MSCs, about 60% less than bone marrow-derived MSCs, about 50% less than bone marrow-derived MSCs, about 40% less than bone marrow-derived MSCs, about 30% less than bone marrow-derived MSCs, about 20% less than bone marrow-derived MSCs, about 10% less than bone marrow-derived MSCs, about 5% less than bone marrow-derived MSCs, or about 1% less than bone marrow-derived MSCs.

In some embodiments, the preparations and compositions of the present disclosure generate a lessened NK cell-mediated immune response upon administration to a subject comprising mis-matched MHC molecules (e.g. mis-matched human leukocyte antigens when the subject is a human), as compared to administration of a composition comprising bone marrow-derived MSCs. In some embodiments, the preparations and compositions of the present disclosure do not generate a NK cell-mediated immune response upon administration to a subject comprising mis-matched MHC molecules (e.g. mis-matched human leukocyte antigens when the subject is a human).

In some embodiments, the composition of γMSCs and/or MSCs may be comprised of less than about 5% CD45+ cells. In some embodiments, the composition of γMSCs and/or MSCs may be comprised of less than about 90%, 80%, 70%, 60%, 50%, 40%, 30%, 20%, or 10% CD45+ cells. In some embodiments, the composition of γMSCs and/or MSCs may be comprised of less than about 0.5% CD45+ to about 10% CD45+. In some embodiments, the composition of γMSCs and/or MSCs may be comprised of less than about 10% CD45+ to about 9% CD45+, about 10% CD45+ to about 8% CD45+, about 10% CD45+ to about 7% CD45+, about 10% CD45+ to about 6% CD45+, about 10% CD45+ to about 5% CD45+, about 10% CD45+ to about 4% CD45+, about 10% CD45+ to about 3% CD45+, about 10% CD45+ to about 2% CD45+, about 10% CD45+ to about 1% CD45+, about 10% CD45+ to about 0.5% CD45+, about 9% CD45+ to about 8% CD45+, about 9% CD45+ to about 7% CD45+, about 9% CD45+ to about 6% CD45+, about 9% CD45+ to about 5% CD45+, about 9% CD45+ to about 4% CD45+, about 9% CD45+ to about 3% CD45+, about 9% CD45+ to about 2% CD45+, about 9% CD45+ to about 1% CD45+, about 9% CD45+ to about 0.5% CD45+, about 8% CD45+ to about 7% CD45+, about 8% CD45+ to about 6% CD45+, about 8% CD45+ to about 5% CD45+, about 8% CD45+ to about 4% CD45+, about 8% CD45+ to about 3% CD45+, about 8% CD45+ to about 2% CD45+, about 8% CD45+ to about 1% CD45+, about 8% CD45+ to about 0.5% CD45+, about 7% CD45+ to about 6% CD45+, about 7% CD45+ to about 5% CD45+, about 7% CD45+ to about 4% CD45+, about 7% CD45+ to about 3% CD45+, about 7% CD45+ to about 2% CD45+, about 7% CD45+ to about 1% CD45+, about 7% CD45+ to about 0.5% CD45+, about 6% CD45+ to about 5% CD45+, about 6% CD45+ to about 4% CD45+, about 6% CD45+ to about 3% CD45+, about 6% CD45+ to about 2% CD45+, about 6% CD45+ to about 1% CD45+, about 6% CD45+ to about 0.5% CD45+, about 5% CD45+ to about 4% CD45+, about 5% CD45+ to about 3% CD45+, about 5% CD45+ to about 2% CD45+, about 5% CD45+ to about 1% CD45+, about 5% CD45+ to about 0.5% CD45+, about 4% CD45+ to about 3% CD45+, about 4% CD45+ to about 2% CD45+, about 4% CD45+ to about 1% CD45+, about 4% CD45+ to about 0.5% CD45+, about 3% CD45+ to about 2% CD45+, about 3% CD45+ to about 1% CD45+, about 3% CD45+ to about 0.5% CD45+, about 2% CD45+ to about 1% CD45+, about 2% CD45+ to about 0.5% CD45+, or about 1% CD45+ to about 0.5% CD45+. In some embodiments, the composition of γMSCs and/or MSCs may be comprised of less than about 10% CD45+, about 9% CD45+, about 8% CD45+, about 7% CD45+, about 6% CD45+, about 5% CD45+, about 4% CD45+, about 3% CD45+, about 2% CD45+, about 1% CD45+, or about 0.5% CD45+. In some embodiments, the composition of γMSCs and/or MSCs may be comprised of less than at least about 10% CD45+, about 9% CD45+, about 8% CD45+, about 7% CD45+, about 6% CD45+, about 5% CD45+, about 4% CD45+, about 3% CD45+, about 2% CD45+, or about 1% CD45+. In some embodiments, the composition of γMSCs and/or MSCs may be comprised of less than at most about 9% CD45+, about 8% CD45+, about 7% CD45+, about 6% CD45+, about 5% CD45+, about 4% CD45+, about 3% CD45+, about 2% CD45+, about 1% CD45+, or about 0.5% CD45+. In some embodiments, the γMSCs and/or MSCs have an antigen profile of about 0% CD45+ cells.

In some embodiments, the composition of γMSCs and/or MSCs may be comprised of at least about 90% CD105+ cells. In some embodiments, the composition of γMSCs and/or MSCs may be comprised of at least about 10%, 20%, 30%, 40%, 50%, 60%, 70%, or 80% CD105+ cells. In some embodiments, the composition of γMSCs and/or MSCs may be comprised of at least about 70% CD105+ cells to about 100% CD105+ cells. In some embodiments, the composition of γMSCs and/or MSCs may be comprised of at least about 100% CD105+ cells to about 95% CD105+ cells, about 100% CD105+ cells to about 94% CD105+ cells, about 100% CD105+ cells to about 93% CD105+ cells, about 100% CD105+ cells to about 92% CD105+ cells, about 100% CD105+ cells to about 91% CD105+ cells, about 100% CD105+ cells to about 90% CD105+ cells, about 100% CD105+ cells to about 85% CD105+ cells, about 100% CD105+ cells to about 80% CD105+ cells, about 100% CD105+ cells to about 75% CD105+ cells, about 100% CD105+ cells to about 70% CD105+ cells, about 95% CD105+ cells to about 94% CD105+ cells, about 95% CD105+ cells to about 93% CD105+ cells, about 95% CD105+ cells to about 92% CD105+ cells, about 95% CD105+ cells to about 91% CD105+ cells, about 95% CD105+ cells to about 90% CD105+ cells, about 95% CD105+ cells to about 85% CD105+ cells, about 95% CD105+ cells to about 80% CD105+ cells, about 95% CD105+ cells to about 75% CD105+ cells, about 95% CD105+ cells to about 70% CD105+ cells, about 94% CD105+ cells to about 93% CD105+ cells, about 94% CD105+ cells to about 92% CD105+ cells, about 94% CD105+ cells to about 91% CD105+ cells, about 94% CD105+ cells to about 90% CD105+ cells, about 94% CD105+ cells to about 85% CD105+ cells, about 94% CD105+ cells to about 80% CD105+ cells, about 94% CD105+ cells to about 75% CD105+ cells, about 94% CD105+ cells to about 70% CD105+ cells, about 93% CD105+ cells to about 92% CD105+ cells, about 93% CD105+ cells to about 91% CD105+ cells, about 93% CD105+ cells to about 90% CD105+ cells, about 93% CD105+ cells to about 85% CD105+ cells, about 93% CD105+ cells to about 80% CD105+ cells, about 93% CD105+ cells to about 75% CD105+ cells, about 93% CD105+ cells to about 70% CD105+ cells, about 92% CD105+ cells to about 91% CD105+ cells, about 92% CD105+ cells to about 90% CD105+ cells, about 92% CD105+ cells to about 85% CD105+ cells, about 92% CD105+ cells to about 80% CD105+ cells, about 92% CD105+ cells to about 75% CD105+ cells, about 92% CD105+ cells to about 70% CD105+ cells, about 91% CD105+ cells to about 90% CD105+ cells, about 91% CD105+ cells to about 85% CD105+ cells, about 91% CD105+ cells to about 80% CD105+ cells, about 91% CD105+ cells to about 75% CD105+ cells, about 91% CD105+ cells to about 70% CD105+ cells, about 90% CD105+ cells to about 85% CD105+ cells, about 90% CD105+ cells to about 80% CD105+ cells, about 90% CD105+ cells to about 75% CD105+ cells, about 90% CD105+ cells to about 70% CD105+ cells, about 85% CD105+ cells to about 80% CD105+ cells, about 85% CD105+ cells to about 75% CD105+ cells, about 85% CD105+ cells to about 70% CD105+ cells, about 80% CD105+ cells to about 75% CD105+ cells, about 80% CD105+ cells to about 70% CD105+ cells, or about 75% CD105+ cells to about 70% CD105+ cells. In some embodiments, the composition of γMSCs and/or MSCs may be comprised of about 100% CD105+ cells, at least about 99% CD105+ cells, about 98% CD105+ cells, about 97% CD105+ cells, about 96% CD105+ cells, about 95% CD105+ cells, about 94% CD105+ cells, about 93% CD105+ cells, about 92% CD105+ cells, about 91% CD105+ cells, about 90% CD105+ cells, about 85% CD105+ cells, about 80% CD105+ cells, about 75% CD105+ cells, or about 70% CD105+ cells. In some embodiments, the composition of γMSCs and/or MSCs may be comprised of at least at least about 100% CD105+ cells, about 95% CD105+ cells, about 94% CD105+ cells, about 93% CD105+ cells, about 92% CD105+ cells, about 91% CD105+ cells, about 90% CD105+ cells, about 85% CD105+ cells, about 80% CD105+ cells, or about 75% CD105+ cells. In some embodiments, the composition of γMSCs and/or MSCs may be comprised of about 99.9% CD105+ cells, about 99.8% CD105+ cells, about 99.7% CD105+ cells, about 99.6% CD105+ cells, about 99.5% CD105+ cells, about 99.4% CD105+ cells, about 99.3% CD105+ cells, about 99.2% CD105+ cells, about 99.1% CD105+ cells, or about 99.0% CD105+ cells.

In some embodiments, the composition of γMSCs and/or MSCs comprise at least about 90% CD166+ cells. In some embodiments, the composition of γMSCs and/or MSCs comprise at least about 10%, 20%, 30%, 40%, 50%, 60%, 70%, or 80% CD166+ cells. In some embodiments, the composition of γMSCs and/or MSCs may be comprised of at least about 70% CD166+ cells to about 100% CD166+ cells. In some embodiments, the composition of γMSCs and/or MSCs may be comprised of at least about 100% CD166+ cells to about 95% CD166+ cells, about 100% CD166+ cells to about 94% CD166+ cells, about 100% CD166+ cells to about 93% CD166+ cells, about 100% CD166+ cells to about 92% CD166+ cells, about 100% CD166+ cells to about 91% CD166+ cells, about 100% CD166+ cells to about 90% CD166+ cells, about 100% CD166+ cells to about 85% CD166+ cells, about 100% CD166+ cells to about 80% CD166+ cells, about 100% CD166+ cells to about 75% CD166+ cells, about 100% CD166+ cells to about 70% CD166+ cells, about 95% CD166+ cells to about 94% CD166+ cells, about 95% CD166+ cells to about 93% CD166+ cells, about 95% CD166+ cells to about 92% CD166+ cells, about 95% CD166+ cells to about 91% CD166+ cells, about 95% CD166+ cells to about 90% CD166+ cells, about 95% CD166+ cells to about 85% CD166+ cells, about 95% CD166+ cells to about 80% CD166+ cells, about 95% CD166+ cells to about 75% CD166+ cells, about 95% CD166+ cells to about 70% CD166+ cells, about 94% CD166+ cells to about 93% CD166+ cells, about 94% CD166+ cells to about 92% CD166+ cells, about 94% CD166+ cells to about 91% CD166+ cells, about 94% CD166+ cells to about 90% CD166+ cells, about 94% CD166+ cells to about 85% CD166+ cells, about 94% CD166+ cells to about 80% CD166+ cells, about 94% CD166+ cells to about 75% CD166+ cells, about 94% CD166+ cells to about 70% CD166+ cells, about 93% CD166+ cells to about 92% CD166+ cells, about 93% CD166+ cells to about 91% CD166+ cells, about 93% CD166+ cells to about 90% CD166+ cells, about 93% CD166+ cells to about 85% CD166+ cells, about 93% CD166+ cells to about 80% CD166+ cells, about 93% CD166+ cells to about 75% CD166+ cells, about 93% CD166+ cells to about 70% CD166+ cells, about 92% CD166+ cells to about 91% CD166+ cells, about 92% CD166+ cells to about 90% CD166+ cells, about 92% CD166+ cells to about 85% CD166+ cells, about 92% CD166+ cells to about 80% CD166+ cells, about 92% CD166+ cells to about 75% CD166+ cells, about 92% CD166+ cells to about 70% CD166+ cells, about 91% CD166+ cells to about 90% CD166+ cells, about 91% CD166+ cells to about 85% CD166+ cells, about 91% CD166+ cells to about 80% CD166+ cells, about 91% CD166+ cells to about 75% CD166+ cells, about 91% CD166+ cells to about 70% CD166+ cells, about 90% CD166+ cells to about 85% CD166+ cells, about 90% CD166+ cells to about 80% CD166+ cells, about 90% CD166+ cells to about 75% CD166+ cells, about 90% CD166+ cells to about 70% CD166+ cells, about 85% CD166+ cells to about 80% CD166+ cells, about 85% CD166+ cells to about 75% CD166+ cells, about 85% CD166+ cells to about 70% CD166+ cells, about 80% CD166+ cells to about 75% CD166+ cells, about 80% CD166+ cells to about 70% CD166+ cells, or about 75% CD166+ cells to about 70% CD166+ cells. In some embodiments, the composition of γMSCs and/or MSCs may be comprised of at least about 100% CD166+ cells, about 95% CD166+ cells, about 94% CD166+ cells, about 93% CD166+ cells, about 92% CD166+ cells, about 91% CD166+ cells, about 90% CD166+ cells, about 85% CD166+ cells, about 80% CD166+ cells, about 75% CD166+ cells, or about 70% CD166+ cells. In some embodiments, the composition of γMSCs and/or MSCs may be comprised of at least at least about 100% CD166+ cells, about 95% CD166+ cells, about 94% CD166+ cells, about 93% CD166+ cells, about 92% CD166+ cells, about 91% CD166+ cells, about 90% CD166+ cells, about 85% CD166+ cells, about 80% CD166+ cells, or about 75% CD166+ cells. In some embodiments, the composition of γMSCs and/or MSCs may be comprised of at least at most about 95% CD166+ cells, about 94% CD166+ cells, about 93% CD166+ cells, about 92% CD166+ cells, about 91% CD166+ cells, about 90% CD166+ cells, about 85% CD166+ cells, about 80% CD166+ cells, about 75% CD166+ cells, or about 70% CD166+ cells.

In some embodiments, the γMSCs are obtained from a matched unrelated donor to the subject or an unmatched donor to the subject. The γMSCs may also be obtained from a donor different from the donor of cells administered in the HCT or from the same donor.

In some embodiments, the composition is formulated to comprise at least about 1×10⁶ γMSCs/kg of ideal body weight or actual body weight of the human subject. In some embodiments, the composition is formulated to comprise at least about 2×10⁶ γMSCs/kg of ideal body weight or actual body weight of the human subject. In some embodiments, the composition is formulated to comprise at least about 5×10⁶ γMSCs/kg of ideal body weight or actual body weight of the human subject. In some embodiments, the composition is formulated to comprise at least about 10×10⁶ γMSCs/kg of ideal body weight or actual body weight of the human subject. In some embodiments, the composition is formulated to comprise at least about 3, 4, 6, 7, 8, 9, 11, 12, 13, 14, 15, 16, 17, 18, 19, or 20×10⁶ γMSCs/kg of ideal body weight or actual body weight of the human subject. In some embodiments, the composition is formulated to comprise at least about 1 to about 5×10⁶ γMSCs/kg, about 6 to about 10×10⁶ γMSCs/kg, about 11 to about 15×10⁶ γMSCs/kg, or about 16 to about 20×10⁶ γMSCs/kg of ideal body weight or actual body weight of the human subject.

Some embodiments include a plurality of compositions. For example, a plurality of compositions may include one composition of γMSCs comprising at least about 2×10⁶ γMSCs/kg of ideal body weight or actual body weight of the human subject and a second composition comprising at least about 5×10⁶ γMSCs/kg of ideal body weight or actual body weight of the human subject. A plurality of compositions may include one composition of γMSCs comprising at least about 2×10⁶ γMSCs/kg of ideal body weight or actual body weight of the human subject and a second composition comprising at least about 10×10⁶ γMSCs/kg of ideal body weight or actual body weight of the human subject. A plurality of compositions may include one composition of γMSCs comprising at least about 5×10⁶ γMSCs/kg of ideal body weight or actual body weight of the human subject and a second composition comprising at least about 10×10⁶ γMSCs/kg of ideal body weight or actual body weight of the human subject. A plurality of compositions may include one composition of γMSCs comprising at least about 2×10⁶ γMSCs/kg of ideal body weight or actual body weight of the human subject, a second composition comprising at least about 5×10⁶ γMSCs/kg of ideal body weight or actual body weight of the human subject, and a third composition comprising at least about 10×10⁶ γMSCs/kg of ideal body weight or actual body weight of the human subject. An additional plurality of compositions may include one composition of γMSCs comprising at least about 1×10⁶ γMSCs/kg of ideal body weight or actual body weight of the human subject, a second composition comprising at least about 2×10⁶ γMSCs/kg of ideal body weight or actual body weight of the human subject, a third composition comprising at least about 5×10⁶ γMSCs/kg of ideal body weight or actual body weight of the human subject, and a fourth composition comprising at least about 10×10⁶ γMSCs/kg of ideal body weight or actual body weight of the human subject. Any of these plurality of compositions may be suitable for hematopoietic cell transplant (HCT). In an embodiment suitable for HCT, the compositions comprise cells obtained from bone marrow and, optionally, selected for CD34+ cells.

Table 6 below shows desirable characteristics of γMSCs compositions for infusion into a patient.

TABLE 6
Desirable characteristics of γMSCs compositions

| | | |
|---|---|---|
| Test | Method | Acceptable Result |

Pre-infusion Data

| | | |
|---|---|---|
| Cell Number | Automated Cell Counter | Required Cell Dose ± 15% |
| Cell Viability | Automated Cell Counter | 85 ± 15% |
| Appearance | Automated Cell Counter | No aggregates |
| Sterility (Day −2) | Microscopy | Gram stain Negative |
| | BacT Alert | 48-hour culture negative |
| Sterility (Day 0) | Microscopy | Gram stain Negative |
| Endotoxin | Endosafe | <2.5 EU/ml |
| Mycoplasma | MycoAlert Assay | None Detected |
| Identity | Flow cytometry | CD45 < 3% |
| | | CD105 > 85% |

Post-Infusion-day Data

| | | |
|---|---|---|
| Sterility (Day −2) | BacT Alert | 14 Day culture Negative |
| Sterility (Day 0) | | 14 Day culture Negative |
| IDO1 Expression | Quantitative PCR | RE > 1 |

### Methods of Treating

Some embodiments of the present disclosure involve use of any of the compositions disclosed herein for preventing or reducing the likelihood of Graft Versus Host Disease (GVHD) or a symptom thereof. The composition may be administered to a subject from about one day before the HCT is administered to at least one day after the subject was administered a hematopoietic stem cell transplant.

In certain aspects, described herein is a method for preventing or reducing the likelihood of GVHD or a symptom thereof in a subject that has been or will be administered a hematopoietic stem cell transplant (HCT), the method comprising administering to the subject an effective amount of interferon γ-primed mesenchymal stromal cells (γMSCs).

In some embodiments, described herein is a method for preventing or reducing the likelihood of GVHD or a symptom thereof in a subject, the method comprising: administering to the subject a hematopoietic stem cell transplant (HCT); and administering to the subject an effective amount of interferon γ-primed mesenchymal stromal cells (γMSCs).

In some embodiments, described herein is a method for preventing or reducing the likelihood of Graft Versus Host Disease (GVHD) or a symptom thereof in a subject that will be administered a hematopoietic stem cell transplant (HCT), the method comprising administering to the subject an effective amount of interferon γ-primed mesenchymal stromal cells (γMSCs).

In some embodiments, the methods described herein comprise the γMSCs being administered to the subject from about one day before the HCT is administered to at least one day after the HCT was administered. The obtained from a deceased donor. The γMSCs may be obtained from a matched unrelated donor to the subject or an unmatched donor to the subject. Additionally, the γMSCs may be obtained from a donor different from the donor of cells administered in the HCT or from the same donor.

An infusion dose can be prepared in a 0.5% HSA+PlasmaLyte-A solution at 4×10⁶ cells/ml and kept at room temperature in an infusion bag or syringe(s) until infused. A sample of the cells, e.g., about 1 to 2×10⁶ γ-MSCs can be collected for detecting microscopic or vegetative contaminating organisms, e.g., bacterial (aerobic & anaerobic) and fungal, endotoxin and mycoplasma presence; the sample can be used for flow cytometry analysis, which may include a determination of viable cell number.

If a clinical product is stored in a syringe, it can be placed on a vertical rotator and maintained at room temperature for not more than 6 hours and if the clinical product is in a bag, it can be placed on a horizontal rotator and maintained at room temperature for not more than 6 hours.

In some embodiments, the amount of γMSCs administered to the subject comprises at least about 1×10⁶ cells/kg of ideal body weight or actual body weight. The subject may also be administered a plurality of doses of γMSCs in amounts from about 1×10⁶ cells/kg to about 10×10⁶ cells/kg of ideal body weight or actual body weight. The subject may be administered a plurality of doses of γMSCs in amounts from about 1×10⁶ cells/kg of ideal body weight or actual body weight to about 2×10⁶ cells/kg of ideal body weight or actual body weight, about 1×10⁶ cells/kg of ideal body weight or actual body weight to about 3×10⁶ cells/kg of ideal body weight or actual body weight, about 1×10⁶ cells/kg of ideal body weight or actual body weight to about 4×10⁶ cells/kg of ideal body weight or actual body weight, about 1×10⁶ cells/kg of ideal body weight or actual body weight to about 5×10⁶ cells/kg of ideal body weight or actual body weight, about 1×10⁶ cells/kg of ideal body weight or actual body weight to about 6×10⁶ cells/kg of ideal body weight or actual body weight, about 1×10⁶ cells/kg of ideal body weight or actual body weight to about 7×10⁶ cells/kg of ideal body weight or actual body weight, about 1×10⁶ cells/kg of ideal body weight or actual body weight to about 8×10⁶ cells/kg of ideal body weight or actual body weight, about 1×10⁶ cells/kg of ideal body weight or actual body weight to about 9×10⁶ cells/kg of ideal body weight or actual body weight, about 1×10⁶ cells/kg of ideal body weight or actual body weight to about 10×10⁶ cells/kg of ideal body weight or actual body weight, about 2×10⁶ cells/kg of ideal body weight or actual body weight to about 3×10⁶ cells/kg of ideal body weight or actual body weight, about 2×10⁶ cells/kg of ideal body weight or actual body weight to about 4×10⁶ cells/kg of ideal body weight or actual body weight, about 2×10⁶ cells/kg of ideal body weight or actual body weight to about 5×10⁶ cells/kg of ideal body weight or actual body weight, about 2×10⁶ cells/kg of ideal body weight or actual body weight to about 6×10⁶ cells/kg of ideal body weight or actual body weight, about 2×10⁶ cells/kg of ideal body weight or actual body weight to about 7×10⁶ cells/kg of ideal body weight or actual body weight, about 2×10⁶ cells/kg of ideal body weight or actual body weight to about 8×10⁶ cells/kg of ideal body weight or actual body weight, about 2×10⁶ cells/kg of ideal body weight or actual body weight to about 9×10⁶ cells/kg of ideal body weight or actual body weight, about 2×10⁶ cells/kg of ideal body weight or actual body weight to about 10×10⁶ cells/kg of ideal body weight or actual body weight, about 3×10⁶ cells/kg of ideal body weight or actual body weight to about 4×10⁶ cells/kg of ideal body weight or actual body weight, about 3×10⁶ cells/kg of ideal body weight or actual body weight to about 5×10⁶ cells/kg of ideal body weight or actual body weight, about 3×10⁶ cells/kg of ideal body weight or actual body weight to about 6×10⁶ cells/kg of ideal body weight or actual body weight, about 3×10⁶ cells/kg of ideal body weight or actual body weight to about 7×10⁶ cells/kg of ideal body weight or actual body weight, about 3×10⁶ cells/kg of ideal body weight or actual body weight to about 8×10⁶ cells/kg of ideal body weight or actual body weight, about 3×10⁶ cells/kg of ideal body weight or actual body weight to about 9×10⁶ cells/kg of ideal body weight or actual body weight, about 3×10⁶ cells/kg of ideal body weight or actual body weight to about 10×10⁶ cells/kg of ideal body weight or actual body weight, about 4×10⁶ cells/kg of ideal body weight or actual body weight to about 5×10⁶ cells/kg of ideal body weight or actual body weight, about 4×10⁶ cells/kg of ideal body weight or actual body weight to about 6×10⁶ cells/kg of ideal body weight or actual body weight, about 4×10⁶ cells/kg of ideal body weight or actual body weight to about 7×10⁶ cells/kg of ideal body weight or actual body weight, about 4×10⁶ cells/kg of ideal body weight or actual body weight to about 8×10⁶ cells/kg of ideal body weight or actual body weight, about 4×10⁶ cells/kg of ideal body weight or actual body weight to about 9×10⁶ cells/kg of ideal body weight or actual body weight, about 4×10⁶ cells/kg of ideal body weight or actual body weight to about 10×10⁶ cells/kg of ideal body weight or actual body weight, about 5×10⁶ cells/kg of ideal body weight or actual body weight to about 6×10⁶ cells/kg of ideal body weight or actual body weight, about 5×10⁶ cells/kg of ideal body weight or actual body weight to about 7×10⁶ cells/kg of ideal body weight or actual body weight, about 5×10⁶ cells/kg of ideal body weight or actual body weight to about 8×10⁶ cells/kg of ideal body weight or actual body weight, about 5×10⁶ cells/kg of ideal body weight or actual body weight to about 9×10⁶ cells/kg of ideal body weight or actual body weight, about 5×10⁶ cells/kg of ideal body weight or actual body weight to about 10×10⁶ cells/kg of ideal body weight or actual body weight, about 6×10⁶ cells/kg of ideal body weight or actual body weight to about 7×10⁶ cells/kg of ideal body weight or actual body weight, about 6×10⁶ cells/kg of ideal body weight or actual body weight to about 8×10⁶ cells/kg of ideal body weight or actual body weight, about 6×10⁶ cells/kg of ideal body weight or actual body weight to about 9×10⁶ cells/kg of ideal body weight or actual body weight, about 6×10⁶ cells/kg of ideal body weight or actual body weight to about 10×10⁶ cells/kg of ideal body weight or actual body weight, about 7×10⁶ cells/kg of ideal body weight or actual body weight to about 8×10⁶ cells/kg of ideal body weight or actual body weight, about 7×10⁶ cells/kg of ideal body weight or actual body weight to about 9×10⁶ cells/kg of ideal body weight or actual body weight, about 7×10⁶ cells/kg of ideal body weight or actual body weight to about 10×10⁶ cells/kg of ideal body weight or actual body weight, about 8×10⁶ cells/kg of ideal body weight or actual body weight to about 9×10⁶ cells/kg of ideal body weight or actual body weight, about 8×10⁶ cells/kg of ideal body weight or actual body weight to about 10×10⁶ cells/kg of ideal body weight or actual body weight, or about 9×10⁶ cells/kg of ideal body weight or actual body weight to about 10×10⁶ cells/kg of ideal body weight or actual body weight.

In some embodiments, the methods described herein comprise a subsequent dose being administered to the subject. The second dose may be administered at an amount greater than the preceding dose. The subject may also receive a plurality of doses comprising at least two doses, three doses, four doses, five doses, six doses, seven doses, eight doses, nine doses, or ten or more doses.

In some embodiments, a first dose comprises about 2×10⁶ γMSCs/kg of ideal body weight or actual body weight, the second dose comprises about 5×10⁶ γMSCs/kg of ideal body weight or actual body weight, and the at least third dose comprises about 10×10⁶ γMSCs/kg of ideal body weight or actual body weight. The first dose may be administered from about one day before the HCT is administered to about one day after the HCT was administered, the second dose may be administered about three days after the HCT was administered, and the at least third dose may be administered from about five days to about thirty days after the HCT was administered. The first dose may be administered the same day as the HCT was administered. The first dose may also be administered about one, two, three, four, five, six, seven, eight, nine, or ten days after the HCT was administered. The second dose may be administered about four, five, six, seven, eight, nine, ten, eleven, twelve, thirteen, fourteen, or fifteen days after the HCT was administered. The at least third dose may be administered about six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty days after the HCT was administered.

In some embodiments, the MSCs may be expanded in culture as described herein prior to being interferon γ-primed. The MSCs may also undergo a primary expansion followed by cryopreservation, as described herein. In some cases, the primary expansion is not followed by cryopreservation and is instead directly undergoes a second expansion. The MSCs may also be thawed and undergo a second expansion, as described herein.

In some embodiments, the MSCs have been thawed to at least about 0° C., 1° C., 2° C., 3° C., 4° C., 5° C., 6° C., 7° C., 8° C., 9° C. or 10° C. In some embodiments, the MSCs have been thawed to between about 0° C. to 10° C., 0° C. to 9° C., 0° C. to 8° C., 0° C. to 7° C., 0° C. to 6° C., 0° C. to 5° C., 0° C. to 4° C., 0° C. to 3° C., 0° C. to 2° C. or 0° C. to 1° C. In some embodiments, the MSCs have been thawed at least about 1 hours, 2 hours, 3 hours, 4 hours, 5 hours, 6 hours, 7 hours, 8 hours, 9 hours, 19 hours, 11 hours, 12 hours, 13 hours, 14 hours, 15 hours, 16 hours, 17 hours, 18 hours, 19 hours, 20 hours, 21 hours, 22 hours, 23 or 24 hours prior to administration to the subject. In some embodiments, the MSCs have been thawed for at least about 1 days, 1.5 days, 2 days, 2.5 days, 3 days, 3.5 days, 4 days, 4.5 days, 6 days, 6.5 days, 7 days, or more than 7 days prior to administration to the subject.

In some embodiments the MSCs are warmed to room temperature before administration to a subject using the methods described herein. In some embodiments, the MSCs are warmed to about body temperature before administration to a subject. In some embodiments, the MSCs are warmed to about 10° C., 11° C., 12° C., 13° C., 14° C., 15° C., 16° C., 17° C., 18° C., 19 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., or 38° C. before administration to a subject.

In some embodiments, the second expansion may last for about seven days. The second expansion may occur for about 1 to about 3 days, about 1 to about 5 days, about 1 to about 9 days, about 1 to about 11 days, about 1 to about 13 days, or about 1 to about 15 days. The second expansion may occur for about 1, 2, 3, 4, 5, 6, 7, 8, 9, 10, 11, 12, 13, 14, or 15 days.

In some embodiments, the MSCs are primed with interferon gamma (IFNγ) during the second expansion. The IFNγ may be added to the expansion media during the final day, the final 2 days, or the final 3 days of the second expansion. The IFNγ may also be added to the expansion media during the final 4 days, the final 5 days, the final 6 days, the final 7 days, the final 8 days, the final 9 days, the final 10 days, the final 11 days, the final 12 days, the final 13 days, or the final 14 days of the second expansion.

In some embodiments, the IFNγ may be present in the expansion medium at a concentration of about 100 U/ml to about 1000 U/ml. The IFNγ may be present in the expansion medium at a concentration of about 10 U/ml to about 500 U/ml, about 10 U/ml to about 1000 U/ml, about 10 U/ml to about 1500 U/ml, about 10 U/ml to about 2000 U/ml, about 50 U/ml to about 500 U/ml, about 50 U/ml to about 1000 U/ml, about 50 U/ml to about 1500 U/ml, about 50 U/ml to about 2000 U/ml, about 100 U/ml to about 500 U/ml, about 100 U/ml to about 1500 U/ml, and about 100 U/ml to about 2000 U/ml. The IFNγ may be present in the expansion medium at a concentration of about 10, 20, 30, 40, 50, 60, 70, 80, 90, 100, 110, 120, 130, 140, 150, 160, 170, 180, 190, 200, 210, 220,230, 240,250,260, 270,280,290, 300, 310, 320, 330, 340, 350, 360, 370, 380, 390, 400, 410, 420,430, 440,450,460, 470,480,490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, or 2000 U/ml.

In some embodiments, the IFNγ may be present in the expansion medium at a concentration of about 1 ng/ml to about 30 ng/ml. The IFNγ may be present in the expansion medium at a concentration of about 1 ng/ml to about 15 ng/ml, 1 ng/ml to about 45 ng/ml, or 1 ng/ml to about 60 ng/ml. The IFNγ may be present in the expansion medium at a concentration of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 ng/ml.

In any of the herein disclosed aspects or embodiments, rather than cryopreserving MSCs or γMSCs and thawing the MSCs (which are subsequently primed) or thawing the γMSCs prior to use (either immediately or after one or more culturing steps), fresh MSCs or fresh γMSCs may be used.

In some cases, when the MSCs were being interferon γ-primed, the culture was not in a hypoxic condition. As examples, the culture did not have decreased levels of O₂, did not have increased levels of CO₂, and/or lacked the presence of a hypoxia mimetic. Decreased levels of O₂ may be from about 1% O₂ to about 5% O₂. Increased levels of CO₂ can be about 5% CO₂ or higher. Illustrative hypoxia mimetics include desferoxamine, cobalt chloride, hydralazine, nickel chloride, diazoxide, and dimethyloxalyglycine.

In some embodiments, the subject is a human. In some embodiments, the human may be a child or an adult. In some embodiments, the subject has myelodysplastic syndrome (MDS) and/or a leukemia. The leukemia may be acute myeloid leukemia (AML), acute lymphoblastic leukemia (ALL), or minimal residual disease (MRD) associated with AML or ALL.

In some embodiments, the γMSCs suppress T cells that underlie GVHD. The GVHD may be acute GVHD (aGVHD) or chronic GVHD (cGVHD). The γMSCs may reduce the likelihood of aGVHD from about day 30 to about day 100 after the HCT was administered. The γMSCs may reduce the likelihood of aGVHD from about day 10 to about day 50, about day 30 to about day 70, about day 30 to about day 130, or about day 50 to about day 150 after the HCT was administered.

In some embodiments, the γMSCs may reduce the likelihood of grade II-IV and/or grade III-IV aGVHD according to the Modified Glucksberg grading scale.

In some embodiments, the γMSCs may reduce the likelihood of cGVHD from about day 100 to about day 365 after the HCT was administered. The γMSCs may reduce the likelihood of cGVHD from about day 50 to about day 150, about day 100 to about day 300, about day 100 to about day 400, or about day 150 to about day 500 after the HCT was administered. The γMSCs may reduce the likelihood of Score 1 to Score 3 cGVHD according to the cGVHD NIH scoring scale.

In some embodiments, the γMSCs may reduce the likelihood of relapse. Relapse may be defined by either morphological or cytogenetic evidence of AML, ALL or MDS and including MRD in ALL consistent with pre-transplant features.

In some embodiments, the γMSCs may increase the likelihood of 1-year GVHD-free/relapse-free survival (GRFS). The γMSCs may increase the likelihood of 2-, 3-, 4-, 5-, 6-, 7-, 8-, 9-, and 10-year GVHD-free/relapse-free survival (GRFS).

In some embodiments, the γMSCs may increase the likelihood of disease-free survival (DFS). DFS may be defined as the minimum time interval from transplant to relapse/recurrence of disease, to death, or to last follow-up.

In some embodiments, the γMSCs may reduce the likelihood of 1- and 2-year non-relapse mortality (NRM). The γMSCs may also reduce the likelihood of 3-, 4-, 5-, 6-, 7-, 8-, 9-, and 10-year NRM.

In some embodiments, the γMSCs may increase the likelihood of overall survival (OS). The γMSCs may reduce the likelihood of primary and/or secondary graft failure. The γMSCs may improve immune reconstitution of 1 year. The γMSCs may also improve immune reconstitution of at least 6 months, 18 months, 2 years, 3 years, 4 years, 5 years, 6 years, 7 years, 8 years, 9 years, or 10 years or more.

In some embodiments, the γMSCs may reduce the time to neutrophil engraftment. Neutrophil engraftment may be defined as achieving a donor derived Absolute Neutrophil Count (ANC)≥500/μL.

In some embodiments, the γMSCs may reduce the time to platelet engraftment at ≥20,000/μl. Platelet engraftment may be defined as the first day of a minimum of three consecutive measurements on different days such that the patient has achieved a platelet count >20,000/μL and >50,000/μL with no platelet transfusions in the preceding seven days.

In some embodiments, the γMSCs may reduce the likelihood of viral (CMV, EBV) reactivation. In some embodiments, the γMSCs may reduce secretion of serum cytokines and/or the presence and/or severity of a cytokine storm. In some embodiments, the γMSCs may reduce the likelihood of a bacterial, fungal, and/or viral infection.

In some embodiments, the methods comprise obtaining the MSCs from one or more vertebral bodies. The MSCs may comprise vertebral bone marrow MSCs (vBM-MSCs), vertebral bone-adherent MSCs (vBA-MSCs), or both. The MSCs may comprise less than about 5% CD45+ cells (e.g., less than less than about 5% CD45+ cells, less than less than about 4% CD45+ cells, less than about 3% CD45+ cells, less than about 2% CD45+ cells, less than about 1% CD45+ cells, or about 0% CD45+ cells), comprises at least about 90% CD105+ cells (e.g., about 90% CD105+ cells, about 91% CD105+ cells, about 92% CD105+ cells, about 93% CD105+ cells, about 94% CD105+ cells, about 95% CD105+ cells, about 96% CD105+ cells, about 97% CD105+ cells, about 98% CD105+ cells, or at least about 99% CD105+ cells), and/or comprises at least about 90% CD166+ cells.

In some embodiments, the HCT comprises cells obtained from bone marrow and, optionally, selected for CD34+ cells.

The present disclosure should be considered as illustrative and not restrictive in character. It is understood that only certain embodiments have been presented and that all changes, modifications and further applications that come within the spirit of the disclosure are desired to be protected.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

### Example 1: Manufacturing γMSCs

This example illustrates a two-step MSC manufacturing process to maximize the immune modularity and tissue regenerative potential of MSCs.

A practical barrier to successful MSC therapy is that freshly thawed, previously cryopreserved cells seem to lack much of the immune modulatory and tissue regenerative potential of freshly expanded cells. The infusion of freshly expanded cells could counteract this. However, the growth kinetics of MSCs in vitro is highly variable; therefore, preparing MSCs to be infused on a preplanned day (e.g. day 1 after HCT) is challenging.

A two-step MSC manufacturing process is used to overcome this obstacle. MSCs are isolated and undergo a primary expansion. The resulting cells are cryopreserved to generate a stock of MSCs. At the appropriate time, the indicated number of cells are thawed and returned to tissue culture for a secondary expansion over a predefined interval (e.g. 7 days). During this secondary expansion, MSCs are primed with IFNγ to reliably yield freshly expanded γMSCs for infusion on the indicated day. This manufacturing process shows that MSCs and γMSCs prepared according to this protocol have the expected characteristics suggesting that these γMSCs do not pose a greater risk to subjects than conventionally prepared cells.

Notably, in some embodiments, when the MSCs were being interferon γ-primed, the culture was not in a hypoxic condition. As examples, the culture did not have decreased levels of O₂, did not have increased levels of CO₂, and/or lacked the presence of a hypoxia mimetic. Decreased levels of O₂ may be from about 1% O₂ to about 5% O₂. Increased levels of CO₂ can be about 5% CO₂ or higher. Illustrative hypoxia mimetics include desferoxamine, cobalt chloride, hydralazine, nickel chloride, diazoxide, and dimethyloxalyglycine.

Bone marrow was obtained from cadaver bones and cryopreserved as described elsewhere in the present disclosure.

Passage 0: One-half bag of the cryopreserved bone marrow is utilized for primary cell culture. The cryopreserved bag was taken out of inventory and the unit was thawed as per the batch record instruction. Approximately 3.9 billion mononuclear cells were plated in six CellBIND® 10-chamber CellSTACKS® and cultured for −14 days in MSC Culture Media. These Passage 0 (P0) cells were cultured for ˜14 days with fresh media changes every 3-4 days.

Passage 1: At >75% confluence, P0 cells were detached using TrypLE™ Select, and ˜410M cells are immediately replated in MSC Culture Media in sixteen CellBIND® 10-chamber CellSTACKS®. These Passage 1 (P1) cells were incubated for 4-5 days and once the flasks were >75% confluent, cells were detached using TrypLE™ Select. P1 cells were resuspended in PlasmaLyte-A+2.5% HSA+5% DMSO at 13M cells/ml and packaged in ˜220×2 mL cryovials at 2 mL per cryovial using an automatic filler for the CellSeal vials. Cells were then cryopreserved and placed into the vapor phase above LN2 in alarm monitored cryogenic tanks for storage at <−140° C. The complete P1 cells testing criteria are presented elsewhere in this disclosure at Table 3.

Passage 2/3: Next, one P1 vial was thawed and plated in one CellBIND® 10-chamber CellSTACK® in MSC Culture Media. These cells became Passage 2 (P2) cells. The P2 cells were cultured for 4-5 days and once the flasks were >75% confluent, cells were detached using TrypLE™ Select. ˜410 million cells were immediately replated in MSC Culture Media in sixteen CellBIND® 10-chamber CellSTACKS®. These then became Passage 3 (P3) cells.

The P3 cells were cultured for 4-5 days and once the flasks were >75% confluent, cells were detached using TrypLE™ Select. P3 cells were resuspended in PlasmaLyte-A+2.5% HSA+5% DMSO at 20M cells/ml and packaged in 5 mL CellSeal closed-system cryovials at 5 mL per cryovial using an automatic filler. Cells were then cryopreserved, which consisted of precooling or equilibration at 4° C. followed by passive cryopreservation at a rate of −1° C./minute to <−80° C. and then moving into vapor phase above LN2 for storage at <−140° C. The complete P3 cells testing criteria were presented in Table 4. The P3 cells constitute the cells which were used for manufacture and final product formulation.

Process Timing and Intermediate Storage: The time elapsed through each culture step was variable, with >75% confluence generally was achieved in 4-5 days. Time elapsed from a culture harvest to the start of the cryopreservation was <6 hours. Upon cryopreservation cells are theoretically stable indefinitely, however, real-time stability studies are ongoing.

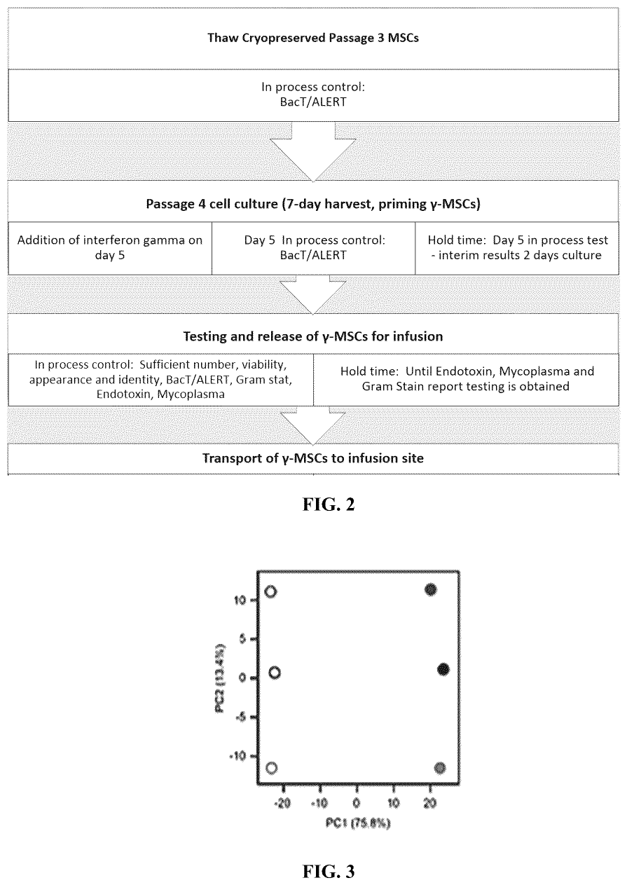

Final Product Manufacturing Process (Secondary Expansion): Processing of the final product for infusion included thawing of the cryopreserved P3 MSCs from Ossium, priming with interferon gamma, and then final formulation. A flow diagram illustrating the process is provided in FIG. 2.

When shipped to a distant site, the vial(s) were shipped in a Cryoport EXP-6 dry vapor shipper (holding temperature validated for up to 10 days). The Cryoport dry vapor shipper was boxed and sent overnight to the distant site. The dry vapor shipper was continuously monitored via Cryoport's Smartpak II® monitoring system. Once at the distant site, the vials were promptly removed from the dry vapor shipper and placed in a liquid nitrogen freezer.

The next step was to remove a vial of the cryopreserved P3 cells and place it into a 37° C. water bath. The vial was kept in the water bath until approximately 80% of ice has melted (approximately 2-3 minutes). Vial was then sprayed with sterile 70% ethanol (EtOH), wiped with sterile wipes and transferred into the biosafety cabinet. The thawed sample was then transferred to a sterile conical tube (50 mL) using a Sexton CellSeal Vial Adapter and syringe. After all P3 cells had been transferred, the cells were diluted with 2 volumes of PlasmaLyte-A+0.5% HSA. The cells were then centrifuged at 500 RCF for 10 minutes at room temperature to form a cell pellet. The supernatant was then removed and the pellet was resuspended in MSC Culture Media. Cell viability and count were then determined using Trypan Blue Exclusion via Manual Counting (or using AO/PI Method via automatic Cellometer). MSCs were then be seeded at 3,000-4,000/cm2 onto eight CellBIND® 5-chamber CellSTACKs® and placed into a 5% CO₂, 37° C., humidified tissue culture incubator. The plating step is considered Day 0 and this cell culture step is considered Passage 4 (P4).

At Day 3, the MSC culture underwent a complete media change to remove expended media and non-adherent cells. MSCs were then returned to the tissue culture incubator for two additional days.

At Day 5, half of the media from each flask was removed and a sample for BAcT/Alert was collected from this expended media from each flask. An aliquot (equating to the half of expended media that was removed) of culture media+50 ng/ml γ-IFN (R&D Systems, Minneapolis, USA) was made and added to each flask according to a standard operating procedure. The interferon gamma primed P4 cells were once again returned to the tissue culture incubator for 2 additional days.

At Day 7, γ-MSCs cell density and morphology were inspected using a bright-field inverted microscope. γ-MSCs were harvested from culture via TrypLE™ Select, washed three times with phosphate buffered saline (PBS), and assessed for cell viability and count. Final product met the preferable features as defined in Table 5.

An infusion dose was prepared in a 0.5% HSA+PlasmaLyte-A solution at 4×10⁶ cells/ml and kept at room temperature in an infusion bag or syringe(s) until infused. Prior to release, approximately 1-2×10⁶ γ-MSCs were taken for a stat Gram Stain, which was examined for any microscopic or vegetative organisms and determination of viable cell number. Testing was initiated for bacterial (aerobic & anaerobic) and fungal, endotoxin and mycoplasma presence, and flow cytometry analysis.

A number of studies have been performed to characterize and evaluate the potential effects of the use of γ-IFN for priming MSCs. Residual levels of γ-IFN, product variability, karyotype, phenotype and potential for in vitro differentiation were evaluated for γ-IFN primed MSCs compared to non-primed MSCs.

To determine whether γ-IFN priming of MSCs imparts the potential for residual activity to the product, residual γ-IFN in the clinical product was examined An ELISA detected the anticipated concentration of γ-IFN in the culture media but no cytokine was detected in the wash media nor in the cell product suspended in infusion media (linear sensitivity, 15 μg/ml).

Thus, subjects should not incur any risk of γ-IFN mediated toxicity as relevant amounts will not be infused into patient (Table 7). γ-IFN was measured by ELISA and reported as pg/ml.

TABLE 7
Residual γ-IFN cells in washing and infusion medium

| | Expanded | Expanded MSCs with |
|---|---|---|
| Test Media | MSCs | (48 h) γ-IFN Priming |
| Fresh Media + γ-IFN, | NA | 5210 |
| 500 U/ml (control) | | |
| Final Culture Medium | 7 | 5239 |
| PBS Plate Rinse | <1 | 86 |
| Wash #1 | <1 | <1 |
| Wash #2 | <1 | <1 |
| Infusion medium (0 h) | <1 | <1 |
| Infusion medium (6 h) | <1 | <1 |

To determine if γ-IFN of MSCs introduced additional variability in the products compared to the intrinsic variability between donors, which has not shown to be problematic, three different preparations of γ-MSCs were assayed with RNA Seq.

Principal component analysis (PCA) using the normalized expression values for the 500 genes with the highest variance, showed distinct clustering of the three γ-MSC samples from MSCs, with the first principal component accounting for 86% of the variance. This analysis also showed a separation of the three donor samples, but this only accounted for 8% of the variance. While the preponderance of variation was due to γ-IFN priming, the cytokine produces a similar variance in each of the patients; thus, γ-IFN priming does not introduce additional inter-donor variation other than what intrinsically exists in MSCs. FIG. 3 demonstrates differential gene expression of MSCs (open shapes) and γ-MSC (filled shapes) from each donor (same color and similar verticality).

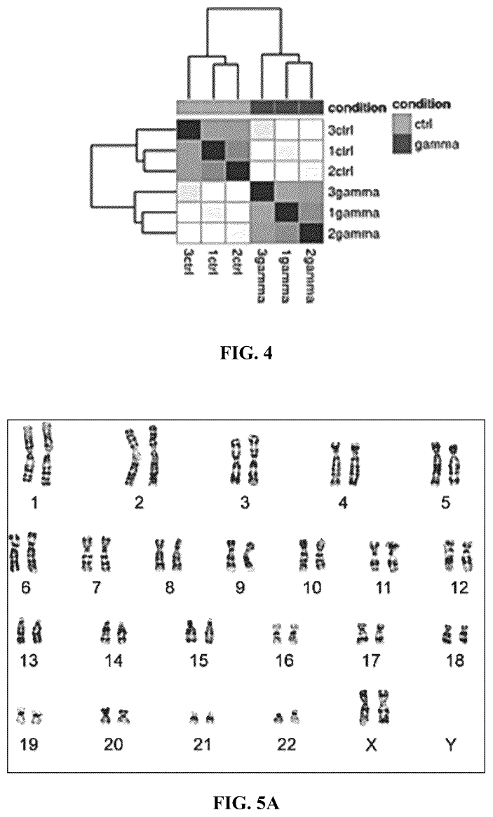

Like PCA, Euclidean distance was calculated to assess the overall similarity between samples. This analysis revealed tight hierarchical clustering of the three treated or untreated samples and distinct separation was clearly visible between the two groups suggesting that y-IFN priming has a similar impact among the donors. FIG. 4 demonstrates overall similarity among the samples. The darker blue indicating a shorter distance, i.e., more similar; lighter blue indicating a greater distance (less similar).

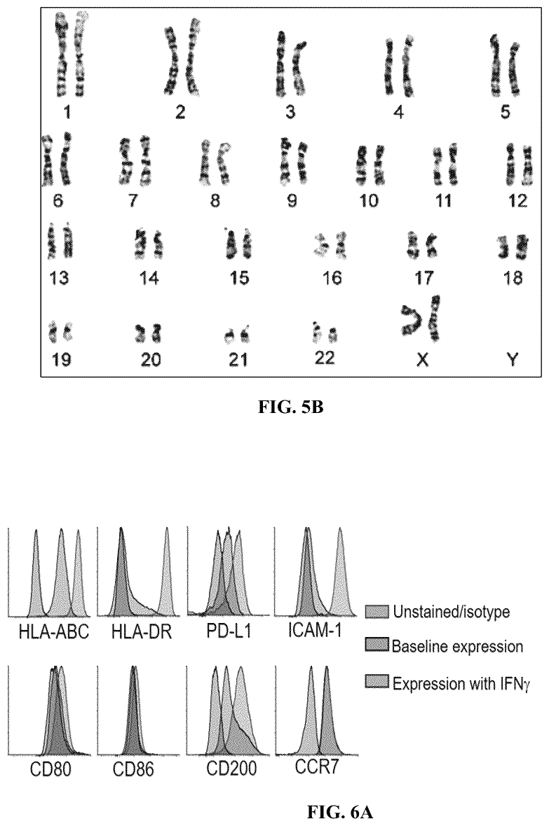

The final MSC product that was to be infused in a patient was assessed for the integrity of the chromosomes (karyotype). Twenty cells in metaphase from the standard MSC preparation and γ-IFN-primed MSCs were examined. All exhibited modal number of 46 chromosomes including two sex chromosomes. No consistent structural or numerical abnormalities were detected. All five cells assessed for G-banding from each preparation displayed a normal pattern. FIG. 5A and FIG. 5B show representative karyotypes of (FIG. 5A) MSCs and (FIG. 5B) γ-IFN-primed MSCs from cells that have undergone primary expansion, cryopreservation, and secondary expansion, and are appropriate to infuse into a subject.

The surface phenotype of the cell products were determined by flow cytometry. γ-IFN-primed MSCs express HLA Class I and II molecules, as well as PD-L1 and PD-L2, but not CD80 or CD86 as expected. MSCs and γ-MSCs underwent in vitro differentiation to osteoblasts, adipocytes, and chondroblasts. Both cell preparations differentiated similarly as determined by Alizarin Red S, Oil Red 0, and Alcian Blue histochemical staining, respectively.

Potency of P1 and P3 cells was evaluated through colony forming unit-fibroblast (CFU-F) analysis (for information/characterization only at P1). For this assay, cells were resuspended in medium and counted using a Cellometer or via Trypan Blue Manual Counting. Next, 50 cells were each pipetted into 2 wells of a 6 well tissue culture treated plate. The plate was incubated for 10-14 days or until colonies are at 80% confluence, refeeding with prewarmed medium every 3-4 days. Once confluent, medium was aspirated, the plate was washed twice with phosphate buffered saline (PBS), and 5 mL of Methanol was added to each well. The cells were allowed to sit for 5 minutes in methanol to fix the cells, then the methanol was removed, and the wells were stained with Crystal Violet for 20 minutes. Finally, the Crystal Violet was removed, and the plates were washed 3 times with DI water, and colonies were counted (a colony was defined as having 50 or more cells). An average of the two wells was taken and the average was used to calculate CFU-F colonies per 1M cells plated.

Table 8 (below) lists the release testing of MSCs has been completed and the acceptable results is presented below.

TABLE 8
Release Testing: Validation Expansions

| | | Acceptable | |
|---|---|---|---|
| Test | Method | Result | Measured Result |
| Cell Number | Automated Cell | Required Cell | 12.2 − 16-fold expansion |
| | Counter | Dose ± 15% | Vial 1 = 1.15 × 10⁹ viable |
| Cell Viability | Automated Cell | 85 ± 15% | 95.80% |
| | Counter | | 95.10% |
| | | | 90.60% |
| Appearance | Automated Cell | No aggregates | No aggregates |
| | Counter | | |
| Sterility (Day −2) | Microscopy | Gram stain | Negative for all three |
| | | Negative | expansions |
| | BacT Alert | 48-hour culture | Negative for all three |
| | | negative | expansions |
| Sterility (Day 0) | Microscopy | Gram stain | Negative for all three |
| | | Negative | expansions |
| Endotoxin | Endosafe | <2.5 EU/ml | <2.5 EU/ml for all three |
| | | | expansions |
| Mycoplasma | MycoAlert Assay | None Detected | None detected for all three |
| | | | expansions |
| Identity | Flow cytometry | CD45 < 3% | 0.0% for all three expansions |
| | | CD105 > 85% | 99.8% for all three |
| | | | expansions |

Post-Infusion-day Data

| | | | |
|---|---|---|---|
| Sterility (Day −2) | BacT Alert | 14 Day culture | Negative for all three |
| | | Negative | expansions |
| Sterility (Day 0) | | 14 Day culture | Negative for all three |
| | | Negative | expansions |
| IDO1 Expression | Quantitative PCR | RE > 1 | RE = 11.5 |
| | | | RE = 26.0 |
| | | | RE = 33.7 |

The method of this example generally follow the process shown in FIG. 1A. However, the steps and ordering thereof as disclosed in this example can be adjusted to follow the processes shown in any of FIG. 11B, FIG. 1C, or FIG. 1D.

### Example 2: Phenotype Analysis of MSC after Exposure to IFNγ

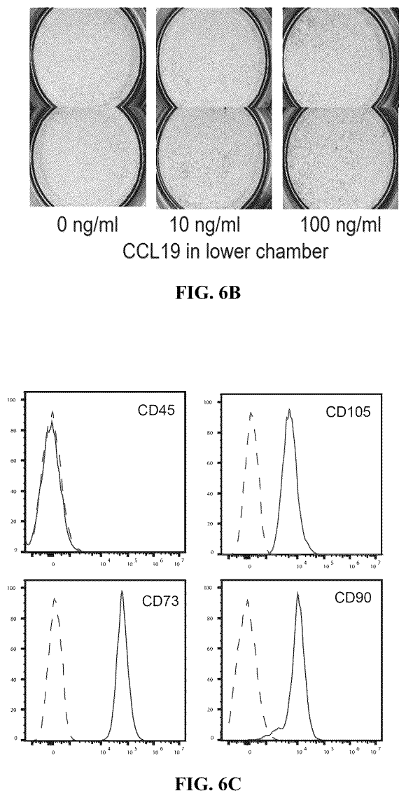

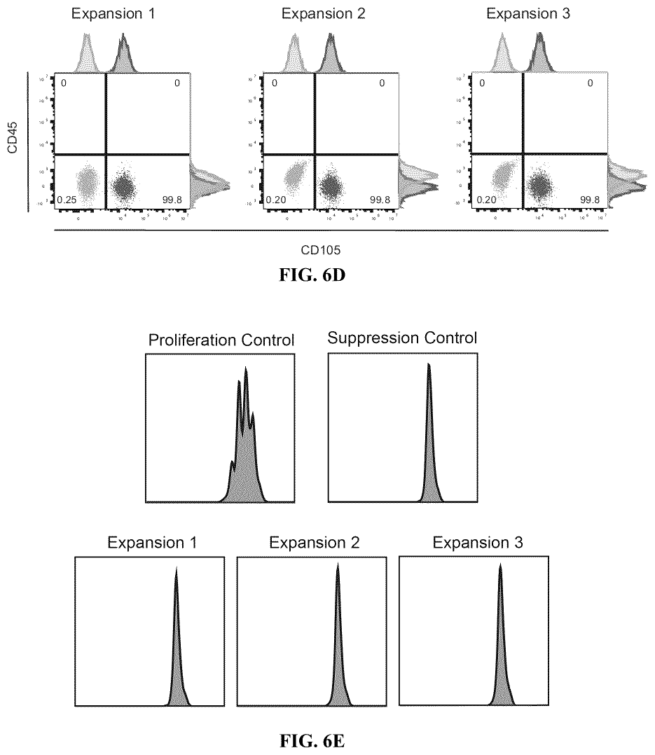

The phenotypes of vBM-MSC and/or vBA-MSCs (collectively MSCs) recovered by the methods described herein, after exposure to IFNγ, were characterized (see FIG. 6A). The observed phenotypes were consistent with the phenotypes previously reported for bone marrow derived MSCs exposed to IFNγ. Moreover, vBA-MSC express low levels of CCR7 which was sufficient to induce migration towards CCL19 in a transwell assay (see FIG. 6B). A flow cytometric analysis of ex vivo expanded, interferon γ primed BM MSCs shows the widely accepted defining antigen expression (FIG. 6C), which shows histograms of flow cytometric analyses of interferon γ primed BM MSCs for the indicated antigens (blue solid line) or isotype control (red dashed line). As shown in FIG. 6C, the γMSC cells lacked expression of CD45 indicating the absence of hematopoietic contamination, and highly express CD105, CD73, and CD90 characteristic of the MSC phenotype. Scatter plots of three γ primed BM-MSCs expansions showed percentages of CD45+ cells of less than 0.25% and percentages of CD105+ cells of 99.8% (See, FIG. 6D). A 10.5-fold expansion over seven days (3.4 population doubling, 48.3 h doubling time), with >99% viability (n=2) was observed with these cells.

The interferon γ primed BM MSCs were able to suppress T cells in vitro. Freshly isolated human T cells were labeled with Cell Trace Far Red (membrane tracking dye, Thermo Fisher) and activated with CD3CD28 Dynabeads® (Thermo Fisher) according to a standard laboratory protocol. The positive control was activated in fresh media while the suppression control was in locally produced (suppressive) γMSC conditioned media. From each validation, MSC expansion and priming, conditioned media was prepared and tested for T cell suppression activity. The cell product for each validation cell expansion suppressed T cell proliferation in response to CD3CD28 bead activation similar to the suppression control (See, FIG. 6E). FIG. 6E demonstrates the dilution of cell trace far red membrane dye (to the left) with expansion compared with a uniform bright signal (to the right) of nondividing cells.

Expression of IDO1 after interferon γ priming was determined and comparison to DDIT4. The expression of DDIT4 is unchanged by interferon γ. The ΔCt was determined by normalizing the cycle threshold cycle (Ct) for IDO1 and DDIT4 to ACTG1 which is consistently expressed in MSCs and γMSCs. The ΔΔCt for IDO1 to DDIT4 and the Relative Expression was determined according to the formula RE=2^{-ΔΔCt}. IDO1 expression was undetectable in MSCs as expected. Any value RE>1 was deemed acceptable. Data is shown below:

| | ΔΔCt | Relative Expression |
|---|---|---|
| Sample 1 of MSCs | −2.279 | 2.8 |
| Sample 2 of MSCs | −1.505 | 4.9 |

Results show equivalence in morphology, phenotype, growth rate and viability, T cell suppression in vitro, and IDO1 expression before and after interferon γ priming of the MSCs. Thus, the γMSC of the present disclosure are suitable for infusion into human patients and for treating various disorders.

### Example 3: Mouse Model Indicating the Ability of Interferon Gamma (IFN γ) to Initiate Murine Mesenchymal Stromal Cell (MSC) Efficacy

In this example, the ability of Interferon γ primed MSCs (γMSC) to suppress Graft Versus Host Disease (GVHD) in mice was determined.

In this example, it was observed that IFN γ was required to initiate murine MSC efficacy to prevent GVHD. Recipients of IFN γ−/−T cells did not respond to MSC treatment and succumbed to GVHD. MSCs pretreated with IFNγ became immediately active and could suppress GVHD more efficiently than naïve (non-treated) MSCs. When given at the time of transplantation, IFNγ-treated MSCs could prevent GVHD mortality (100% survival p=0.006). MSC activation was also dependent on the magnitude of IFNγ exposure. Increased IFN exposure (maximum responses seen with exposure to 500 IU of IFN γ) leads to increased MSC-mediated suppression of GVHD.

It was determined that mouse models of GVHD show different levels of severity depending on many factors including MHC matching and the number of T cells transplanted. A fully MHC-mismatched model of GVHD was used, which is a more extreme immunologic disparity that would ever be used clinically. BALB/c mice (H-2^{d}) were transplanted with a hematopoietic graft comprised of 5×10⁶ T-cell depleted marrow cells and 5×10⁵ T cells freshly obtained from C57BL/6 mice (H-2b). 5×10⁵ murine or human γMSCs were infused on days 1 and 3.

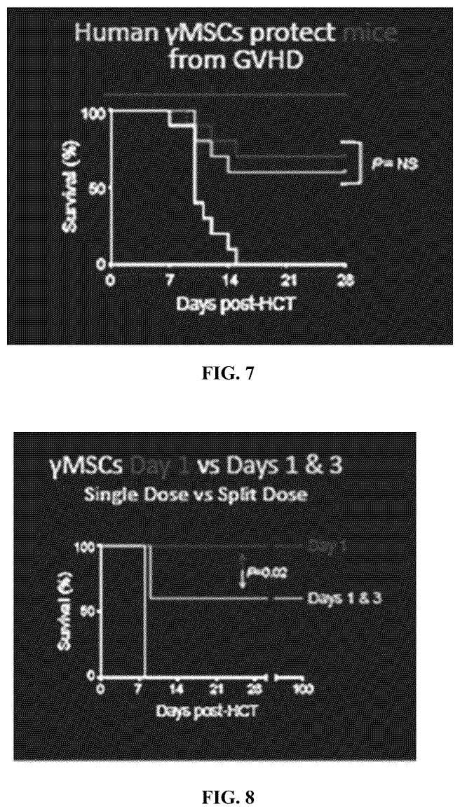

As illustrated in FIG. 7, the median survival of the control mice was 10 days with all mice succumbing to the effects of GVHD by day 17. By contrast, 60% of mice receiving murine γMSCs survived for 100 days (P=0.003). Importantly, human γMSCs suppressed GVHD in the murine model equivalent to murine cells (60% vs 70% survival, P=NS).

After intravenous infusion, it was demonstrated that γMSCs rapidly traffic to the spleen and presumably other secondary lymphoid organs where transplanted, and naïve donor T cells are activated by host antigen presenting cells (APCs) initiated acute GVHD. Here, the γMSCs co-localized amongst the proliferating T cells. On day 3 (2 days after the first γMSC infusion, but before the second infusion), a nearly 2 log reduction in the number of donor CD4+ and CD8+ T cells in the mouse spleen of γMSCs treated mice compared to untreated controls was observed. On day 6, however, the T cell populations were similar to controls. These data suggested that the day 3 γMSC dose may have had little effect in the secondary lymphoid organs (SLO) and conceivably did not contribute to the suppression of GVHD. To test this idea, the outcome of transplanted mice receiving human γMSCs on days 1 and 3 (5×10⁵ cells/dose) with animals receiving γMSCs on day 1 only (1×10⁶ cells) were compared.

As illustrated in FIG. 8, mice receiving the total cell dose given as a single infusion showed 100% survival for 100 days compared with 60% survival of mice receiving the divided dose split into two infusions. These data indicated that efficacy lies in the day 1 infusion as well as a γMSC dose-dependency of GVHD suppression. The data also showed that γMSCs are most potent when infused on day 1 after hematopoietic cell transplantation (HCT), compared to a repeated infusion approach (day 1 and 3). Moreover, a larger dose was markedly more effective.

This example also showed that γMSCs are phagocytes and engulfment viable, and activate T cells by efferocytosis, the specialized form of phagocytosis by which macrophages clear apoptotic cells. This observation indicates that the overall process of efferocytosis includes induction of an immune suppressive microenvironment albeit the molecular pathways are poorly understood. This example also showed that γMSC efferocytosis is indispensable for efficient prophylaxis of GVHD in the murine model.

### Example 4: Cryopreserved MSCs from HLA Identical Sibling Donors Successfully Transplanted in Humans

This example illustrates that cryopreserved MSCs from HLA identical sibling donors can be transplanted in humans.

Cryopreserved MSCs from HLA identical sibling donors, cultured in fetal bovine serum were successfully infused in 46 adult transplant recipients. In this example, there were no toxicities and greater than 90% manufacturing success rate was reported. In a randomized single center study, 30 patients were randomized to receive either MSCs along with stem cells or only stem cells. The feasibility of the manufacturing process was only 66.7%. The incidence of the acute GVHD and chronic GVHD was less in the treatment group. However, there was a high rate of disease relapse and reduced disease-free survival (DFS) in the treatment arm. Cryopreserved MSCs were also successfully infused in recipients of HLA identical peripheral blood stem cell transplants (PBSC).

### Example 5: MSCs Successfully Infused in Non-HLA Identical Transplants

This example illustrates that peri-transplant infusion of MSCs is safe and feasible in non-HLA identical transplants.

In this example, MSCs from the bone marrow of either matched sibling or haploidentical donors were infused in unrelated HCT. Successful engraftment was noted in all three recipients with a history of graft failure. MSCs were infused in recipients of single unit cord and T cell depleted mobilized PBSCs.

Adult patients who underwent a non-myeloablative transplant using HLA mismatched unrelated donors, also received third party MSCs. This cohort had decreased incidence of acute GVHD, reduced 100-day non-relapse mortality and overall mortality in comparison to historical controls.

### Example 6: Phase I Dose-Escalation Study Assessing the Manufacturing Feasibility, Maximal Tolerated Dose, and Safety of Freshly Expanded IFN-γ Primed MSCs as GVHD Prophylaxis in Children and Adults Undergoing Allogeneic Hematopoietic Cell Transplantation for Hematological Malignancies and Myelodysplasia

This example is for evaluating the safety and feasibility of peri-transplant infusion of freshly expanded interferon gamma primed MSCs in adult and pediatric patients undergoing HCT for acute leukemia and Myelodysplastic Syndrome (MDS).

A minimum of 4 and a maximum of 45 patients are included, including both adult and pediatric patients, in the dose escalation and expansion cohorts. This includes 1 dose de-escalation in the pediatric cohort if the maximum tolerated dose (MTD) in adults is not tolerated in children. The duration of the study is 5 years. The duration of accrual is up to 3 years. Each subject remains on study up to 2 years after HCT.

To estimate the MTD, the rolling-6 design is used. The rolling-6 design allows for accrual of two to six patients concurrently onto a dose level. Decisions as to which dose level to enroll a patient is passed on the number of patients currently enrolled and evaluable, the number of patients experiencing dose limiting toxicities (DLTs), and the number of patients at risk of developing a DLT. No intra-patient escalation is allowed.

Table 9 provides possible scenarios for a rolling-6 design for a given dosing stratum.

TABLE 9
dose escalation

| Scenario | | | | | |
|---|---|---|---|---|---|
| | # of | | | Decision when Next Subject is | |
| # of | Subjects | | # of Subjects | Enrolled | |
| Subjects | with | # of Subjects | with Toxicity | Not at the highest | At the highest |
| Enrolled | DLT | without DLT | data pending | dose level | dose level |
| 2 | 0, 1 | Any | Any | Stay | — |
| 2 | 2 | 0 | 0 | De-escalate | — |
| 3 | 0 | 0, 1, 2 | 3, 2, 1 | Stay | — |
| 3 | 0 | 3 | 0 | Escalate | |
| 3 | 1 | 0, 1, 2 | 2, 1, 0 | Stay | |
| 3 | ≥2 | Any | Any | De-escalate | |
| 4 | 0 | 0, 1, 2, 3 | 4, 3, 2, 1 | Stay | Stay |
| 4 | 0 | 4 | 0 | Escalate | Stay |
| 4 | 1 | 0, 1, 2, 3 | 3, 2, 1, 0 | Stay | Stay |
| 4 | ≥2 | Any | Any | De-escalate | De-escalate |
| 5 | 0 | 0, 1, 2, 3, 4 | 4, 3, 2, 1 | Stay | Stay |
| 5 | 0 | 5 | 0 | Escalate | Stay |
| 5 | 1 | 0, 1, 2, 3, 4 | 4, 3, 2, 1 | Stay | Stay |
| 5 | ≥2 | Any | Any | De-escalate | De-escalate |
| 6 | 0 | 0, 1, 2, 3, 4 | 4, 3, 2, 1 | Suspend | Suspend |
| 6 | 0 | 5, 6 | 0, 1 | Escalate | MTD |
| 6 | 1 | 0, 1, 2, 3, 4 | 4, 3, 2, 1 | Suspend | Suspend |
| 6 | 1 | 5 | 0 | Escalate | MTD |
| 6 | ≥2 | Any | Any | De-escalate | De-escalate |

* Stay refers to staying at the current dosing level. De-escalate refers to dropping to the next lowest dosing level, escalate refers to moving to the next highest dosing level. Suspend refers to stopping enrollment until enough patients have been evaluated to make a decision about the next possible patient.

Based on the table above, dose escalation occurs if 0 out of 3-6 or at most 1 out of 6 evaluable patients experience a DLT when treated at a dose level. If 2 out of 2-6 patients experience a DLT, the dose is considered too toxic and above the MTD. Once a dose is determined too toxic, no escalation to higher dose levels is allowed.

The MTD is defined as the highest dose level at which 6 patients are treated with at most 1 patient experiencing a DLT, and the next higher dose level has been determined to be too toxic. Once the MTD is determined in adults, and safety data of the investigational product is evaluated and accepted, children <18 are enrolled at the MTD dose level, following the rolling 6 design. If >1 out of 6 patients experience a DLT, then the dose is de-escalated to the next lower level, per Table 9.

Monitoring of safety continues during the expansion cohort. After 3 evaluable patients (in the pediatric and adult expansion cohorts separately), if the raw percentage of DLTs ever exceeds 33%, accrual halts.

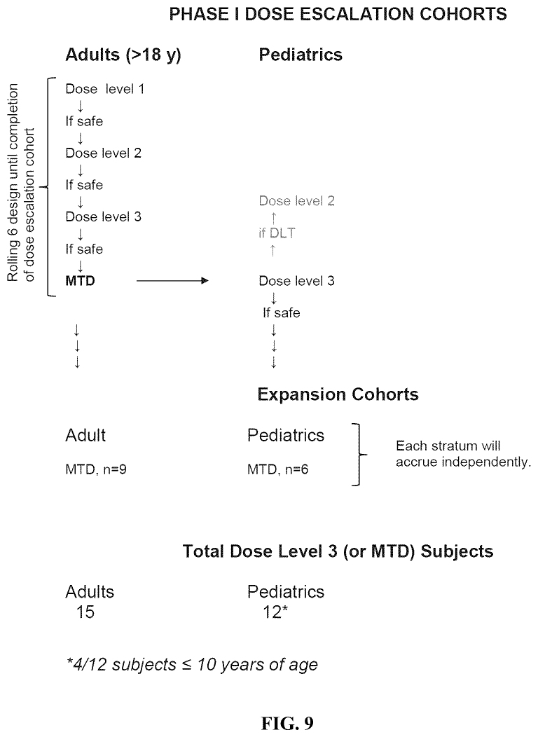

As illustrated in FIG. 9, treatment starts at dose level 1. Dose escalation proceeds as described above and in Table 9. Treatment of the first two subjects in each dose escalation cohort is staggered. The second subject is infused with the study product until the first subject completes the DLT observation interval (minimum of 21 days).

TABLE 10
Dose levels

| Dose Level | MSC dose* |
|---|---|
| Dose level −1 | 1 × 10⁶ cells/kg |
| Dose level 1 | 2 × 10⁶ cells/kg |
| Dose level 2 | 5 × 10⁶ cells/kg |
| Dose level 3 | 10 × 10⁶ cells/kg |

*Based on ideal body weight or actual body weight

The cell dose levels illustrated in Table 10 are based on the subject's ideal body weight or actual body weight (IBW). Since γMSCs traffic to the secondary lymphoid organs, which does not expand with increasing adiposity, the IBW is a more appropriate index of the total mass of lymphoid tissue. Initially, adult subjects receive a single infusion of third party, freshly ex vivo expanded, IFNγ-primed MSCs at a dose of 2×10⁶ cells/kg of ideal body weight or actual body weight on Day +1 (the day after infusion of the hematopoietic cell graft). The dose is escalated to 5×10⁶ and then 10×10⁶ cells/kg according to dose escalation procedure described above. In absence of any dose limiting toxicity, 10×10⁶ cells/kg is accepted as the maximal dose.

For children 1-18 years of age, the IBW is calculated as follows: IBW (kg)=[height (cm)²×1.65]÷1000. If the subject is male and less than 60 inches, then the IBW is calculated as follows: IBW (kg)=39+2.27×(Height (inches)−60). If the subject is female and less than 60 inches, the IBW is calculated as follows: IBW (kg)=45.5+2.3×(Height (inches)−60). For male adults, the IBW is calculated as follows: IBW (kg)=50.0+2.3×(Height (inches)−60). For female adults, the IBW is calculated as follows: IBW (kg)=45.5+2.3×(Height (inches)−60).

Third party MSCs are prepared for infusion from a healthy donor(s) and cryopreserved in a repository specifically designated for this protocol. At the appropriate time, an aliquot of MSCs appropriate for the size of the subject are thawed, returned to tissue culture and maintained until infusion. Over the final 2 days of tissue culture, interferon γ (500 U/ml) is added to the media.

MSCs are isolated from freshly harvested healthy donor(s) bone marrow and expanded under GMP conditions. MSCs are expanded following standard operating practices and cryopreserved as numerous aliquots in individual bags. At the designated time prior to the planned cell infusion, an appropriate number of cells are thawed and placed into tissue culture, and supplemented with IFNγ 2 days prior to infusion. After about 48 hours, the IFN-primed MSCs are collected and after meeting the release criteria, are sent to the subject's bedside for infusion.

Notably, in some embodiments, when the MSCs were being interferon γ-primed, the culture was not in a hypoxic condition. As examples, the culture did not have decreased levels of O₂, did not have increased levels of CO₂, and/or lacked the presence of a hypoxia mimetic. Decreased levels of O₂ may be from about 1% O₂ to about 5% O₂. Increased levels of CO₂ can be about 5% CO₂ or higher. Illustrative hypoxia mimetics include desferoxamine, cobalt chloride, hydralazine, nickel chloride, diazoxide, and dimethyloxalyglycine.

MSCs are intravenously infused through a central line or a large bore peripheral IV using standard blood product tubing within 4 hours of release. The product is infused by IV push or syringe pump over 5-15 minutes. No medications or fluids are given through the catheter that is used for MSC infusion. Subjects may be given diphenhydramine (1 mg/kg, 50 mg max dose) and acetaminophen (10 mg/kg, 650 mg max dose) within 60 minutes of the cell infusion.

Infusion of IFNγ-primed MSCs occurs around the time of stem cell infusion, the period during which maximum tissue damage, exposure of host antigens and activation of donor T cells is known to occur.

On the day of the MSCs infusion, the following procedures/assessments are performed: (1) assessment of clinical condition: Patients with active uncontrolled infections should not receive MSC infusion. The clinical condition is discussed with the study PI or designee prior to final determination; (2) vital signs (BP, HR, respiratory rate, and temperature) are measured within 10 minutes prior to the γMSC infusion (time 0) and then 15 minutes, 30 minutes, 1 and 2 hours after the start of the γMSC infusion. Oxygen saturation is continuously monitored by pulse oximetry for at least 30 minutes prior to MSC infusion and until 2 hours after the start of the MSC infusion; and (3) the SaO₂ must be stable at ≥92% (based on the highest reading after equilibration has occurred) for at least 30 minutes prior to administration of the γMSC. Patients requiring supplemental oxygen are excluded from receiving the study product.

MSC administration is stopped if: (1) the patient has symptoms or signs of respiratory compromise such as tachypnea, cyanosis, complains of shortness of breath, etc. regardless of the oximetry reading, OR (2) SaO₂ decreases to <85% over a continuous period of 3 to 5 minutes whether or not the patient has symptoms of respiratory distress. If the SaO₂ is <85% in the absence of symptoms or signs, the accuracy of the pulse oximeter reading is confirmed by using another machine, relocating the sensor, or testing the device on a healthy person.

Infusion of the MSCs is stopped at the discretion of the treating physician if there is an adverse event that the treating physician believes is related to the MSCs or there is an issue with MSCs infusion, or the patient withdraws consent.

No other medications are given during the MSC infusion unless determined medically necessary by the treating physician.

Throughout the trial, data is gathered. Graft samples are assessed by flow cytometry according to the immune reconstitution panel. MNC are isolated from samples obtained from the graft and from the subject within 12 hours prior to graft infusion. These cells are assayed for the capacity to induce apoptosis in a sample of the γMSCs which were infused. Analysis of inflammatory cytokines and markers, including GVHD specific markers, are assessed by multiplex analysis. Samples are obtained on day 7, and then at the onset of acute GVHD.

Samples are obtained from subjects on days 7, 14, 28, 60, 100, 180 and 365 for assessment of clonal expansion of infused T cells. Samples of blood are obtained on days 60, 100, 180, 270, and 365 for assessment of immune reconstitution. The volume of the blood samples is 10 ml plus waste blood for all subjects.

Immune reconstitution (flow cytometry) and functional assessments (T cell/NK cell functional assays) are evaluated on days 100, 180, and 365. The absolute count of Th1, Th2, Th17 and T_{reg} differentiated CD4+ cells is determined during early timepoints (day +7, 14,60, 100, 180, 365) to assess the effect of γMSCs, and early changes associated with development of GVHD.

Vaccine response is performed on pediatric patients only. Pneumococcal vaccination (Prevnar 13) is administered per standard of care after day 180. Baseline samples are obtained, then at 2 additional points at 7-14 days, and 6-8 weeks after vaccine administration. Serotype-specific antibody levels and serotype-specific opsonophagocytic activity (OPA) are assessed at the same time points.

T cell gene signature is determined via single cell RNA sequencing. T cells are sorted by flow cytometry, with a minimum of 500 CD4+ and CD8+ cells analyzed per time point. RNA expression profiles confirm T cell identity (CD3+CD4+ or CD3+CD8+). T cell gene signatures are compared among patients with or without GVHD.

At the conclusion of the trial, the total body of data is considered in an effort to (i) determine the feasibility of freshly preparing and IFNγ priming allogeneic MSCs for infusion as GvHD prophylaxis, (ii) identify the toxicities associated with such processed MSCs, and (iii) gain insight as to the efficacy for GvHD prophylaxis.

### Example 7: Preventing Reducing the Likelihood of GVHD with IFNγ Primed MSC

This example provides methods for preventing or reducing the likelihood of Graft Versus Host Disease (GVHD) or a symptom thereof in a subject that has been administered a hematopoietic stem cell transplant (HCT); the method comprises administering to the subject an effective amount of interferon γ-primed mesenchymal stromal cells (γMSCs), as described in Example 1.

This example also provides methods for preventing or reducing the likelihood of Graft Versus Host Disease (GVHD) or a symptom thereof in a subject. The method comprising steps of administering to the subject a hematopoietic stem cell transplant (HCT); and administering to the subject an effective amount of interferon γ-primed mesenchymal stromal cells (γMSCs), as described in Example 1. When the MSCs were being interferon γ-primed, the MSCs were not in a culture in a hypoxic condition.

This example further provides methods for preventing or reducing the likelihood of Graft Versus Host Disease (GVHD) or a symptom thereof in a subject that will be administered a hematopoietic stem cell transplant (HCT). The method comprising administering to the subject an effective amount of interferon γ-primed mesenchymal stromal cells (γMSCs) as described in Example 1. When the MSCs were being interferon γ-primed, the MSCs were not in a culture in a hypoxic condition.

In this example, compositions comprising γMSCs (as described in Example 1) are administered via intravenously infusion with a dose of γMSCs in the amounts from about 1×10⁶ cells/kg to about 10×10⁶ cells/kg of ideal body weight or actual body weight.

In some cases, the γMSCs is administered to the subject from about one day before the HCT is administered to at least one day after the HCT was administered. The γMSCs may be obtained from a deceased donor. The γMSCs may be obtained from a matched unrelated donor to the subject or an unmatched donor to the subject. Additionally, the γMSCs may be obtained from a donor different from the donor of cells administered in the HCT or from the same donor.

In this example, the subject is a human child or a human adult.

In some cases, the subject is administered a subsequent dose of γMSCs. The second dose may be administered at an amount greater than the preceding dose. The subject may also receive a plurality of doses comprising at least two doses, three doses, four doses, five doses, six doses, seven doses, eight doses, nine doses, or ten or more doses.

In some cases, a first dose comprises about 2×10⁶ γMSCs/kg of ideal body weight or actual body weight, the second dose comprises about 5×10⁶ γMSCs/kg of ideal body weight or actual body weight, and the at least third dose comprises about 10×10⁶ γMSCs/kg of ideal body weight or actual body weight. The first dose may be administered from about one day before the HCT is administered to about one day after the HCT was administered, the second dose may be administered about three days after the HCT was administered, and the at least third dose may be administered from about five days to about thirty days after the HCT was administered. The first dose may be administered the same day as the HCT was administered. The first dose may also be administered about one, two, three, four, five, six, seven, eight, nine, or ten days after the HCT was administered. The second dose may be administered about four, five, six, seven, eight, nine, ten, eleven, twelve, thirteen, fourteen, or fifteen days after the HCT was administered. The at least third dose may be administered about six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty days after the HCT was administered.

### Example 8: Supplements to Priming MSCs with IFNγ

This example illustrates supplements to priming MSCs with IFNγ.

Following the steps described in Example 1, during the secondary expansion, MSCs are primed with IFNγ and serum starved and/or temperature shocked. More specifically, to enact serum starvation, during the last two days or the last day of culturing, human platelet lysate (hPL) is omitted from the culturing medium. Alternately, or additionally, during the final two hours or final hour of culturing, the temperature of the cell culture is raised to about 40° C., to enact temperature shock.

### Example 9: Alternates to Priming MSCs with IFNγ

This example illustrates alternates to priming MSCs with IFNγ.

Following the steps described in Example 1, during the secondary expansion, rather than priming MSCs with IFNγ, the MSCs are serum starved and/or temperature shocked. More specifically, to enact serum starvation, during the last two days or the last day of culturing, human platelet lysate (hPL) is omitted from the culturing medium. Alternately, or additionally, during the final two hours or final hour of culturing, the temperature of the cell culture is raised to about 40° C., to enact temperature shock.

### Example 10: Preventing Reducing the Likelihood of GVHD with IFNγ Primed MSC

This example provides methods for preventing or reducing the likelihood of Graft Versus Host Disease (GVHD) or a symptom thereof in a subject that has been administered a hematopoietic stem cell transplant (HCT); the method comprises administering to the subject an effective amount of interferon γ-primed mesenchymal stromal cells (γMSCs), as described in Example 8.

This example also provides methods for preventing or reducing the likelihood of Graft Versus Host Disease (GVHD) or a symptom thereof in a subject. The method comprising steps of administering to the subject a hematopoietic stem cell transplant (HCT); and administering to the subject an effective amount of interferon γ-primed mesenchymal stromal cells (γMSCs), as described in Example 8. When the MSCs were being interferon γ-primed, the MSCs were not in a culture in a hypoxic condition.

This example further provides methods for preventing or reducing the likelihood of Graft Versus Host Disease (GVHD) or a symptom thereof in a subject that will be administered a hematopoietic stem cell transplant (HCT). The method comprising administering to the subject an effective amount of interferon γ-primed mesenchymal stromal cells (γMSCs) as described in Example 8. When the MSCs were being interferon γ-primed, the MSCs were not in a culture in a hypoxic condition.

In this example, compositions comprising γMSCs (as described in Example 8) are administered via intravenously infusion with a dose of γMSCs in the amounts from about 1×10⁶ cells/kg to about 10×10⁶ cells/kg of ideal body weight or actual body weight.

In some cases, the γMSCs is administered to the subject from about one day before the HCT is administered to at least one day after the HCT was administered. The γMSCs may be obtained from a deceased donor. The γMSCs may be obtained from a matched unrelated donor to the subject or an unmatched donor to the subject. Additionally, the γMSCs may be obtained from a donor different from the donor of cells administered in the HCT or from the same donor.

In this example, the subject is a human child or a human adult.

In some cases, the subject is administered a subsequent dose of γMSCs. The second dose may be administered at an amount greater than the preceding dose. The subject may also receive a plurality of doses comprising at least two doses, three doses, four doses, five doses, six doses, seven doses, eight doses, nine doses, or ten or more doses.

In some cases, a first dose comprises about 2×10⁶ γMSCs/kg of ideal body weight or actual body weight, the second dose comprises about 5×10⁶ γMSCs/kg of ideal body weight or actual body weight, and the at least third dose comprises about 10×10⁶ γMSCs/kg of ideal body weight or actual body weight. The first dose may be administered from about one day before the HCT is administered to about one day after the HCT was administered, the second dose may be administered about three days after the HCT was administered, and the at least third dose may be administered from about five days to about thirty days after the HCT was administered. The first dose may be administered the same day as the HCT was administered. The first dose may also be administered about one, two, three, four, five, six, seven, eight, nine, or ten days after the HCT was administered. The second dose may be administered about four, five, six, seven, eight, nine, ten, eleven, twelve, thirteen, fourteen, or fifteen days after the HCT was administered. The at least third dose may be administered about six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty days after the HCT was administered.

### Example 11: Preventing Reducing the Likelihood of GVHD with IFNγ Primed MSC

This example provides methods for preventing or reducing the likelihood of Graft Versus Host Disease (GVHD) or a symptom thereof in a subject that has been administered a hematopoietic stem cell transplant (HCT); the method comprises administering to the subject an effective amount of interferon γ-primed mesenchymal stromal cells (γMSCs), as described in Example 9.

This example also provides methods for preventing or reducing the likelihood of Graft Versus Host Disease (GVHD) or a symptom thereof in a subject. The method comprising steps of administering to the subject a hematopoietic stem cell transplant (HCT); and administering to the subject an effective amount of interferon γ-primed mesenchymal stromal cells (γMSCs), as described in Example 9. When the MSCs were being interferon γ-primed, the MSCs were not in a culture in a hypoxic condition.

This example further provides methods for preventing or reducing the likelihood of Graft Versus Host Disease (GVHD) or a symptom thereof in a subject that will be administered a hematopoietic stem cell transplant (HCT). The method comprising administering to the subject an effective amount of interferon γ-primed mesenchymal stromal cells (γMSCs) as described in Example 9. When the MSCs were being interferon γ-primed, the MSCs were not in a culture in a hypoxic condition.

In this example, compositions comprising γMSCs (as described in Example 9) are administered via intravenously infusion with a dose of γMSCs in the amounts from about 1×10⁶ cells/kg to about 10×10⁶ cells/kg of ideal body weight or actual body weight.

In some cases, the γMSCs is administered to the subject from about one day before the HCT is administered to at least one day after the HCT was administered. The γMSCs may be obtained from a deceased donor. The γMSCs may be obtained from a matched unrelated donor to the subject or an unmatched donor to the subject. Additionally, the γMSCs may be obtained from a donor different from the donor of cells administered in the HCT or from the same donor.

In this example, the subject is a human child or a human adult.

In some cases, the subject is administered a subsequent dose of γMSCs. The second dose may be administered at an amount greater than the preceding dose. The subject may also receive a plurality of doses comprising at least two doses, three doses, four doses, five doses, six doses, seven doses, eight doses, nine doses, or ten or more doses.

In some cases, a first dose comprises about 2×10⁶ γMSCs/kg of ideal body weight or actual body weight, the second dose comprises about 5×10⁶ γMSCs/kg of ideal body weight or actual body weight, and the at least third dose comprises about 10×10⁶ γMSCs/kg of ideal body weight or actual body weight. The first dose may be administered from about one day before the HCT is administered to about one day after the HCT was administered, the second dose may be administered about three days after the HCT was administered, and the at least third dose may be administered from about five days to about thirty days after the HCT was administered. The first dose may be administered the same day as the HCT was administered. The first dose may also be administered about one, two, three, four, five, six, seven, eight, nine, or ten days after the HCT was administered. The second dose may be administered about four, five, six, seven, eight, nine, ten, eleven, twelve, thirteen, fourteen, or fifteen days after the HCT was administered. The at least third dose may be administered about six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty days after the HCT was administered.

### Example 12: Fresh, not Previously Frozen IFNγ Primed MSC

In any of the above examples, rather than cryopreserving MSCs or γMSCs and thawing the MSCs (which are subsequently primed) or thawing the γMSCs prior to use (either immediately or after one or more culturing steps), fresh MSCs or fresh γMSCs may be used.

1. A composition comprising interferon γ-primed human mesenchymal stromal cells (γMSCs) for preventing or reducing the likelihood of Graft Versus Host Disease (GVHD) or a symptom thereof in a human subject that has been administered a hematopoietic stem cell transplant (HCT), wherein when the MSCs were being interferon γ-primed, the MSCs were not in a culture in a hypoxic condition.

2. The composition of claim 1, wherein the MSCs are obtained from one deceased human.

3. The composition of claim 1, wherein the MSCs are obtained from one or more vertebral bodies.

4. The composition of claim 1, wherein the MSCs comprise vertebral bone marrow MSCs (vBM-MSCs), vertebral bone-adherent MSCs (vBA-MSCs), or both.

5. The composition of claim 1, wherein the MSCs were expanded in culture prior to being interferon γ-primed.

6. The composition of claim 1, wherein the MSCs have undergone a primary expansion followed by cryopreservation or the MSCs have undergone a primary expansion that was not followed by cryopreservation and instead underwent a second expansion.

7. The composition of claim 6, wherein the cryopreserved MSCs were thawed and then underwent a second expansion.

8. The composition of claim 6, wherein the second expansion occurred for about seven days.

9. The composition of claim 6, wherein the MSCs were primed with interferon gamma (IFNγ) during the second expansion.

10. The composition of claim 9, wherein during the final day, the final two days, or the final three days of the second expansion, IFNγ was added to the expansion media.

11. The composition of claim 9, wherein the IFNγ is present in the second expansion media at a concentration of from about 100 U/ml to about 1000 U/ml and/or from about 1 to about 30 ng/ml.

12. The composition of claim 1, wherein the composition comprises less than about 5% CD45+ cells, less than about 4% CD45+ cells, less than about 3% CD45+ cells, less than about 2% CD45+ cells, less than about 1% CD45+ cells, or about 0% CD45+ cells.

13. The composition of claim 1, wherein the composition comprises at least about 90% CD105+ cells, at least about 91% CD105+ cells, at least about 92% CD105+ cells, at least about 93% CD105+ cells, at least about 94% CD105+ cells, at least about 95% CD105+ cells, at least about 96% CD105+ cells, at least about 97% CD105+ cells, at least about 98% CD105+ cells, or at least about 99% CD105+ cells.

14. The composition of claim 1, wherein the composition comprises at least about 90% CD166+ cells.

15. The composition of claim 1, wherein the γMSCs are obtained from a matched unrelated donor to the subject or an unmatched donor to the subject.

16. The composition of claim 1, wherein the γMSCs are obtained from a donor different from the donor of cells administered in the HCT or from the same donor.

17. The composition of claim 1, wherein composition is formulated to comprise at least about 1×10⁶ γMSCs/kg of ideal body weight or actual body weight of the human subject.

18. The composition of claim 1, wherein composition is formulated to comprise at least about 2×10⁶ γMSCs/kg of ideal body weight or actual body weight of the human subject.

19. The composition of claim 1, wherein composition is formulated to comprise at least about 5×10⁶ γMSCs/kg of ideal body weight or actual body weight of the human subject.

20. The composition of claim 1, wherein composition is formulated to comprise at least about 10×10⁶ γMSCs/kg of ideal body weight or actual body weight of the human subject.

21. A method for preventing or reducing the likelihood of Graft Versus Host Disease (GVHD) or a symptom thereof in a subject that has been administered a hematopoietic stem cell transplant (HCT), the method comprising administering to the subject an effective amount of interferon γ-primed mesenchymal stromal cells (γMSCs), wherein when the MSCs were being interferon γ-primed, the MSCs were not in a culture in a hypoxic condition.

22. The method of claim 21, wherein the γMSCs are administered at least one day after the HCT was administered.

23. The method of claim 21, wherein the γMSCs are obtained from a matched unrelated donor to the subject or an unmatched donor to the subject.

24. The method of claim 1, wherein the γMSCs are obtained from a donor different from the donor of cells administered in the HCT or from the same donor.

25. The method of claim 21, wherein the cells administered in the HCT and/or the γMSCs are obtained from a deceased donor.

26-70. (canceled)

71. A method for preventing or reducing the likelihood of Graft Versus Host Disease (GVHD) or a symptom thereof in a subject, the method comprising:
administering to the subject a hematopoietic stem cell transplant (HCT); and
administering to the subject an effective amount of interferon γ-primed mesenchymal stromal cells (γMSCs),
wherein when the MSCs were being interferon γ-primed, the MSCs were not in a culture in a hypoxic condition.

72. The method of claim 71, wherein the γMSCs are administered from about one day before the HCT is administered to at least one day after the HCT was administered.

73. The method of claim 71, wherein the γMSCs are obtained from a matched unrelated donor to the subject or an unmatched donor to the subject.

74. The method of claim 71, wherein the γMSCs are obtained from a donor different from the donor of cells administered in the HCT or from the same donor.

75. The method of claim 71, wherein the cells administered in the HCT and/or the γMSCs are obtained from a deceased donor.

76-154. (canceled)
